(19) United States
(12) Patent Application Publication
(10) Pub. No.: US 2021/0228433 A1
(43) Pub. Date: Jul. 29, 2021

(54) ROLLATOR

(71) Applicant: 9208-5216 QUEBEC INC., Danville (CA)

(72) Inventors: Jean-Marc Landry, Val-Joli (CA); Michel Landry, Stoke (CA)

(73) Assignee: 9208-5216 QUEBEC INC., Danville (CA)

(21) Appl. No.: 16/319,700

(22) PCT Filed: Jul. 24, 2017
(86) PCT No.: PCT/CA2017/050885

§ 371 Date: Jan. 22, 2019

(30) Foreign Application Priority Data
Jul. 22, 2016 (CA) 2936983

(51) Int. Cl. A61H 3/04

(52) U.S. Cl. CPC A61H 3/04; A61H 2003/046; A61H 2201/0161; A61H 2201/1635; A61H 2201/5071; A61H 2201/0176; A61H 2201/1633

(57) ABSTRACT

A rollator for mobility assistance to a user is provided. The rollator includes a chassis foldable in a compacted configuration, wheels rotatably mounted to the chassis, wheel braking assemblies engageable with the wheels, and a pair of handgrips mountable to the chassis and operatively connected to the chassis. Both handgrips have to be configured in a handgrip displacement configuration to configure the wheel braking assemblies into a displacement configuration of the wheels and wherein both wheel braking assemblies are simultaneously in the displacement configuration. The rollator can include a seat assembly which can be configured in a raised configuration and a seated configuration. In the seated configuration, a pressure equal to or greater than a seat pressure threshold has to be applied on the seat for the wheel braking assemblies to be configured in the displacement configuration.

### CROSS-REFERENCE TO RELATED APPLICATIONS

This application claims the benefit of Canadian Patent Application No. 2,936,983, filed on Jul. 22, 2016 and entitled “SMART-ROLLATOR WITH EVERYDAY LIFE ADAPTED CHASSIS, FALL DETECTION SYSTEM, AUTOMATIC BRAKING AND ANTI-ROLL BACK SYSTEMS, MANUFACTURING METHOD AND USAGES THEREOF”. This Canadian informal patent application is incorporated herein by reference in its entirety.

### TECHNICAL FIELD OF THE INVENTION

The present invention relates to the field of mobility assistance devices, and, more particularly, to a rollator, also known as a wheeled (or rolling) walker.

### BACKGROUND

Many persons, due to age or disability, have difficulty in walking without a walking aid. Wheeled walkers, also known as rollators, are widely used to assist in mobility. A wheeled walker typically has a frame mounted on four wheels and a pair of rearwardly extending handle bars which the user can grip for support while walking. The user positions himself between the handle bars behind the wheeled walker and pushes the walker forward. The wheels permit the user to roll the walker smoothly over the ground thereby avoiding the laborious action of picking up and moving a non-wheeled walker in step-by-step fashion. The handle bars can be provided with brake levers that when squeezed by the user to actuate a wheel braking assembly.

Wheeled walkers are routinely equipped with a seating surface that permits the user to rest in the sitting position. The seating surface is usually positioned transversely between the handle bars within the wheel base of the walker to offer a stable platform for sitting. In order to use the seating surface, the user must turn around and sit down in the rearward facing direction, opposite to the normal walking direction, with his feet resting on the ground. The wheel braking assembly can be engageable in a braking configuration to maintain braking engagements with the wheels to prevent the walker from rolling while the user is sitting.

Occasionally, the rollator user can be too tired to continue walking and can require the assistance of a caregiver to pursue their displacement. Conventional wheeled walkers are not adapted to support a seated user and, simultaneously, be pushed by a caregiver. Typically, there is not enough spacing between the rollator user, seated in a rearward facing position between the handlebars, and the caregiver pushing the rollator to perform walking steps without interfering with the user's feet. US patent application no. 2004/0118640 addresses this issue by describing a rollator equipped with a strap-liked backrest that permits a two-way sitting for the user. When sitting at the front of the rollator, the space cleared at the rear of the rollator allow a caregiver to push the rollator with sufficient clearance between him and the user for his leg displacement. However, to adopt this seated configuration, the user has to walk around the rollator to be able to sit in the transport chair position which increases the risk of a fall when walking around the rollator.

The use of a rollator should not limit the accessibility to public spaces. Rollators should provide a constant safety support in most situations. In most existing rollators, the frame and the seat are provided forwardly of the user in the walking configuration. Therefore, the walking user is located behind the rollator and is provided only with forward support. Because the frame is located mainly forwardly of the walking user, it may be difficult for the user to carry out simple tasks at a kitchen counter, for instance, without having to put his rollator behind him to be closer to the working surface. However, while positioning the rollator behind him, the user is provided with no or limited support. Similarly, access to narrow non-adapted public toilets may be difficult or impossible with conventional rollators due to the limited spacing. Often, the user must leave the rollator outside the toilet cabinet and take hold of anything he can to sit on the toilet, thereby increasing the risk of fall. Rollator users take up to three times the space of a normal walking person. In narrow and limited spaces, such as in elevators, this may make the rollator user uncomfortable.

In addition, the field of view of a rollator user pushing his rollator is typically reduced by the rollator seat in a seated configuration. More particularly, the rollator seat in the folded configuration creates a blind spot in front of the rollator that increases the potential of rolling over an object. Giving the fact that rollator users may suffer from sight limitations, the field of view immediately located in front of them should be as clear as possible.

Rollators can also be used as a rest chair. In a seated configuration, the user often uses his feet to propel himself to continue his displacement. In the seated configuration, most rollators are by default in a parking mode, with the brakes engaged with the wheels to prevent displacement thereof. Furthermore, the braking system is manually operated. Some rollators include a brake bypass to voluntarily deactivate the brakes when a user uses his rollator as a rest chair. However, when the brakes are disengaged from the wheels, the user, using his rollator in the seated position, will become at risk of falling when attempting to stand up.

As mentioned above, rollators are typically equipped with brakes that allow the user to stop and/or control the rolling device. Existing brake assemblies are configured to brake the rollator either by pulling brake handgrips or by stepping on brake pedals. More particularly, some of the rollators commercially available are equipped with a manually-activated braking assembly having three modes of operation: a parking mode, a slowing down mode, and a rolling (walking) mode. To stop or control the rollator, the user must intentionally configure the rollator into the parking mode. However, when dealing with memory impaired users or new users, it may happen that they forget to configure the rollator into the parking mode, i.e. apply the brakes, or they may not have the reflex to apply them appropriately, i.e. without a sufficient pressure or good timing. Most of the time, when feeling a fall, the rollator user may not think about applying the brakes but may take hold on whatever he has in hand (the handgrip) to take support or transfer his weight in an attempt to retain his balance or his stability. The perfect braking device should be able to automatically detect whenever the user needs a firm and safe support, it should automatically detect when the user is at risk of falling and should react accordingly. Therefore, there may be a need to develop a braking assembly that automatically applies brakes when a hazardous situation is encountered by the rollator user.

Several prior art patents and patent applications tried to provide safer braking assemblies for rollators. For instance, US patent application no. 2013/0062845 discloses a rollator with a safety brake device wherein the rollator is configured in a braked or detained state when no pressure is applied on the handgrip unit. When the handgrip unit is pivoted from a non-depressed position to a depressed position, a stop member is configured from a braking position to a free position wherein the wheels are freely rotatable. Therefore, when not used, the brakes of the rollator are engaged with the wheels. However, there is no mechanism to prevent falls. More particularly, as mentioned above, typically, when a person feels like he is falling, he will take hold with his hand(s), thereby transferring his body weight onto his hand(s). With the US2013/0062845 rollator, when transferring the body weight onto the handgrip unit, the rollator users could deactivate the brakes and the body weight could then apply a forward pressure on the rollator making it to roll away from the user and a fall could occur.

US patent application no. 2012/0090926 describes a safety braking device system for a hand-pushed rollator having a 3-mode breaking device: the parking mode, the slowing down mode, and the rolling (walking) mode. The slowing down mode includes the activation of a friction brake to slow down the rollator by squeezing a handle mounted under the handgrip. The parking mode is automatically applied when the lever is released, thereby the rollator is by default in the parking mode. Thus, the rollator user needs to apply a constant pressure on the lever to stay in the rolling mode. Application of a constant pressure can be a problem for those suffering from muscular weaknesses or having problem squeezing things. Also, a user must always think about applying the proper pressure on the lever which can cause frustration with some users suffering from mental illnesses. On the other hand, it may sometimes be difficult to teach the pressure management to a user. It would be suitable that the rollator should detect whether or not to apply the brakes, leaving the user with a minimum of operations and decisions to think about.

U.S. Pat. No. 6,338,355 discloses a safety brake type rollator including two gear-type brakes that are linked through a rod-type link to a pivoting bar which allows the brakes to work together as a whole. The pivoting bar is activated by two levers mounted under each one of the handgrips. When either of the levers is pulled, both brakes are simultaneously deactivated allowing the rollator to move freely. As in US patent application no. 2012/0090926, the rollator user needs to apply a constant pressure on the lever to stay in the rolling mode again, which may be non-suitable for some users. Moreover, the breaking system of this rollator, which is by default activated, can be deactivated using only one hand, which increases the instability and fall risks.

Therefore, there may be a need for an automatic braking system, hazard detection system and fall prevention system using typical reflex movement of a rollator user.

In view of the above, there is a need for a rollator which would be able to overcome or at least minimize some of the above-discussed prior art concerns.

### BRIEF SUMMARY OF THE INVENTION

It is therefore an aim of the present invention to address at least some of the above-mentioned issues.

According to a general aspect, there is provided a rollator for mobility assistance to a user. The rollator comprises:
a substantially U-shaped chassis including:
   a front portion and two lateral side portions, each one of the lateral side portions extending rearwardly from a respective end of the front portion to define a seat/walker receiving area;
at least one pair of wheels rotatably mounted to the chassis; and
a seat assembly including a seat pivotally mounted to the chassis and selectively configurable in a raised configuration and a seated configuration wherein the seat extends substantially horizontally in the seat/walker receiving area, the seat/walker receiving area being unobstructed when the seat is configured in the raised configuration.

In an embodiment, the rollator further comprises a wheel braking system engageable with the at least one pair of wheels and configurable in a displacement configuration allowing rotation of the wheels and a braking configuration wherein the wheel braking system is engaged with at least one of the at least one pair of wheels to prevent rotation thereof. The seat assembly can be operatively connected to the wheel braking system, wherein when the seat is configured in the seated configuration, the wheel braking system is configured in the braking configuration if a pressure applied on the seat is lower than a seat pressure threshold. In an embodiment, when the seat is configured in the seated configuration, the wheel braking system is configured in the displacement configuration if a pressure equal to or greater than the seat pressure threshold is applied thereon. In an embodiment, the seat is further pivoted downwardly towards a horizontally-extending configuration when a pressure equal to or greater than the seat pressure threshold is applied to the seat.

In an embodiment, the rollator further comprises two handgrip assemblies, each one including a pivotable handgrip operatively connected to the wheel braking system, the handgrips being selectively configurable in a handgrip displacement configuration and in a handgrip braking configuration, and wherein the wheel braking system is configured in the braking configuration in the raised configuration of the seat unless the two handgrips are configured in the handgrip displacement configuration. Each one of the handgrips can be selectively configurable in:
a raised configuration and a lowered configuration, wherein, if the seat is configured in the raised configuration, configuration of at least one of the handgrips in the raised configuration configures the wheel braking system in the braking configuration and configuration of at least one of the handgrips in the lowered configuration with a pressure applied thereon equal to or greater than a handgrip pressure threshold configures the wheel braking system in the braking configuration; and configuration of both of the handgrips in the lowered configuration with a pressure applied thereon being lowered than the handgrip pressure threshold configures the wheel braking system in the displacement configuration.

The handgrips can be configured in the raised configuration when no pressure is applied thereon.

In an embodiment, the at least one pair of wheels comprises a pair of fixed wheels mounted to rear ends of the lateral side portions of the chassis and the rollator further comprises swivel wheels rotatably mounted to the front portion of the chassis.

In an embodiment, the lateral side portions are pivotally mounted to the respective end of the front portion and selectively configurable in an operative configuration wherein they are spaced-apart from one another to define the seat/walker receiving area inbetween and a compacted configuration wherein they are superposed to each other and extend substantially parallel to the front portion. The seat can be configured in the raised configuration in the compacted configuration of the lateral side portions and is located between the front portion and the lateral side portions of the chassis.

In an embodiment, the wheel braking system further comprises a wheel braking assembly controller mounted to the chassis and wheel braking assemblies having wheel stoppers engageable with a respective one of the wheels, each one of the wheel stoppers being selectively configurable in the displacement configuration wherein it is disengaged from the respective one of the wheels to allow rotation thereof and in the braking configuration wherein it is engaged with the respective one of the wheels to prevent rotation thereof; and wherein the wheel braking assembly controller operatively connects the handgrips to the wheel braking assemblies.

The two handgrip assemblies can comprise at least one handgrip frame pivotally mounted to the chassis and having the handgrips pivotally mounted thereto. The at least one handgrip frame can be selectively configurable in an operative configuration wherein at least one of the handgrips is located rearwardly of the front portion of the chassis and a frontward configuration wherein at least one of the handgrip is located forwardly of the front portion of the chassis.

In an embodiment, the rollator further comprises a walking re-education assembly including a frame removably engageable with the chassis and a ring, the frame having two lateral side portions spaced-apart from one another and extending substantially parallel to a respective one of the lateral side portions of the chassis when engaged therewith, the ring being attached to the two lateral side portions of the frame and extending between, the ring being configured to partially support a weight of the user during a rehabilitation process. The fixed wheels can comprise at least two sets of fixed wheels, each one of the sets of fixed wheels including at least one left-side fixed wheel and one right-side fixed wheel and each one of the sets can be characterized by a different wheel diameter, the fixed wheels being detachably, selectively and rotatably securable to the lateral side portions of the chassis.

In an embodiment, the wheel braking system further comprises gear disks, each one being secured to the respective one of the wheels and having a plurality of triangular-shaped teeth extending peripherally and the respective one of the wheel stoppers is engageable with the respective one of the gear disks in the braking configuration to prevent rotation of the respective one of the wheels.

In an embodiment, the seated configuration of the seat comprises a first seated configuration and a second seated configuration. In the first and second seated configurations, the seat can extend substantially horizontally in the seat/walker receiving area and the second seated configuration can be reached when a pressure equal to or greater than a seat pressure threshold is applied on the seat configured in the first seated configuration. The seat assembly can be operatively connected to the wheel braking system, wherein when the seat is configured in the second seated configuration, the wheel braking system is configured in the displacement configuration and, when the seat is configured in the first seated configuration, the wheel braking system is configured in the braking configuration.

In an embodiment, the chassis further comprises at least one accessory receiving tube extending substantially vertically.

According to another general aspect, there is provided a rollator for mobility assistance to a user. The rollator comprises:
a chassis including:
   a front portion and two lateral side portions, each one of the lateral side portions extending rearwardly from a respective end of the front portion to define a seat/walker receiving area;
at least one pair of wheels rotatably mounted to the chassis; and
a handgrip assembly including a handgrip frame pivotally mounted to the chassis and including a lateral side portion extending substantially in a same plane than a respective one of the lateral side portions of the chassis, the lateral side portion of the handgrip frame including a handgrip mounted thereto, the handgrip frame being configurable in an operative configuration wherein the handgrip is located rearwardly of the front portion of the chassis and a frontward configuration wherein the handgrip is located forwardly of the front portion of the chassis.

In an embodiment, the rollator can further comprise a pair of secondary handgrips mounted to a respective one of the lateral side portions and adjacent to a rear end thereof, the secondary handgrips extending below the handgrip mounted to the pivotable handgrip frame in the operative configuration thereof.

In an embodiment, the rollator can further comprise a seat assembly and a wheel braking system. The seat assembly can include a seat pivotally mounted to the chassis and selectively configurable in a raised configuration and a seated configuration wherein the seat extends substantially horizontally in the seat/walker receiving area, the seat/walker receiving area being unobstructed when the seat is configured in the raised configuration. The wheel braking system can be engageable with the at least one pair of wheels and configurable in a displacement configuration allowing rotation of the wheels and a braking configuration wherein the wheel braking system is engaged with at least one of the at least one pair of wheels to prevent rotation thereof. The seat assembly can be operatively connected to the wheel braking system, wherein when the seat is configured in the seated configuration, the wheel braking system is configured in the braking configuration if a pressure applied on the seat is lower than a seat pressure threshold.

In an embodiment, when the seat is configured in the seated configuration, the wheel braking system is configured in the displacement configuration if a pressure equal to or greater than the seat pressure threshold is applied thereon and the seat is further pivoted downwardly towards a horizontally-extending configuration when a pressure equal to or greater than the seat pressure threshold is applied to the seat. In an embodiment, the handgrip is pivotally mounted to the handgrip frame and is operatively connected to the wheel braking system, the handgrip being selectively configurable in:
a raised configuration and a lowered configuration, wherein, if the seat is configured in the raised configuration, configuration of the handgrip in the raised configuration configures the wheel braking system in the braking configuration and configuration of the handgrip in the lowered configuration with a pressure applied thereon equal to or greater than a handgrip pressure threshold configures the wheel braking system in the braking configuration.

The handgrip can be configured in the raised configuration when no pressure is applied thereon.

In an embodiment, the at least one pair of wheels comprises a pair of fixed wheels mounted to rear ends of the lateral side portions of the chassis and the rollator further comprises swivel wheels rotatably mounted to the front portion of the chassis.

In an embodiment, the lateral side portions are pivotally mounted to the respective end of the front portion and selectively configurable in an operative configuration wherein they are spaced-apart from one another to define the seat/walker receiving area inbetween and a compacted configuration wherein they are superposed to each other and extend substantially parallel to the front portion. The seat can configured in the raised configuration in the compacted configuration of the lateral side portions and can be located between the front portion and the lateral side portions of the chassis.

According to a further general aspect, there is provided a rollator for mobility assistance to a user, the rollator comprising:
a chassis including:
   a front portion and two lateral side portions pivotally mounted to a respective end of the front portion, the lateral side portions being configurable in an operative configuration wherein they are spaced-apart from one another and extend substantially parallel to each other to define a seat/walker receiving area inbetween and a compacted configuration wherein they are superposed to each other and extend substantially parallel to the front portion; and
at least one pair of wheels rotatably mounted to the chassis.

In an embodiment, the lateral side portions extend substantially normal to the front portion in the operative configuration.

In an embodiment, the rollator further comprises a seat assembly and a wheel braking system. The seat assembly includes a seat pivotally mounted to the chassis, operatively connected to the wheel braking system, and selectively configurable in a raised configuration and a seated configuration wherein the seat extends substantially horizontally in the seat/walker receiving area. The wheel braking system can be engageable with the at least one pair of wheels and configurable in a displacement configuration allowing rotation of the wheels and a braking configuration wherein the wheel braking system is engaged with at least one of the at least one pair of wheels to prevent rotation thereof. The seat can be configured in the raised configuration in the compacted configuration of the lateral side portions of the chassis and can be located between the front portion and the lateral side portions of the chassis. In an embodiment, when the seat is configured in the seated configuration, the wheel braking system is configured in the displacement configuration if a pressure equal to or greater than a seat pressure threshold is applied thereon, and is configured in the braking configuration otherwise.

In an embodiment, the rollator further comprises two handgrip assemblies, each one including a pivotable handgrip operatively connected to the wheel braking system, the handgrips being selectively configurable in a handgrip displacement configuration and in a handgrip braking configuration, and wherein the wheel braking system is configured in the braking configuration in the raised configuration of the seat unless the two handgrips are configured in the handgrip displacement configuration. Each one of the handgrips can be selectively configurable in a raised configuration and a lowered configuration. If the seat is configured in the raised configuration, configuration of at least one of the handgrips in the raised configuration can configure the wheel braking system in the braking configuration and configuration of at least one of the handgrips in the lowered configuration with a pressure applied thereon equal to or greater than the handgrip pressure threshold can configure the wheel braking system in the braking configuration; and configuration of both of the handgrips in the lowered configuration with a pressure applied thereon being lowered than the handgrip pressure threshold configures the wheel braking system in the displacement configuration. The handgrips can be configured in the raised configuration when no pressure is applied thereon.

In an embodiment, the at least one pair of wheels comprises a pair of fixed wheels mounted to rear ends of the lateral side portions of the chassis and the rollator further comprises swivel wheels rotatably mounted to the front portion of the chassis.

In an embodiment, the seat/walker receiving area is unobstructed when the seat is configured in the raised configuration.

In an embodiment, the two handgrip assemblies comprise at least one handgrip frame pivotally mounted to the chassis and having the handgrips pivotally mounted thereto, the at least one handgrip frame being selectively configurable in an operative configuration wherein at least one of the handgrips is located rearwardly of the front portion of the chassis and a frontward configuration wherein at least one of the handgrips is located forwardly of the front portion of the chassis.

According to still another general aspect, there is provided a rollator for mobility assistance to a user. The rollator comprises:
a chassis including:
   a front portion and two lateral side portions, each one of the lateral side portions extending rearwardly from a respective end of the front portion to define a seat/walker receiving area;
at least one pair of wheels rotatably mounted to the chassis;
a wheel braking assembly comprising: at least one gear disk secured to a respective one of the wheels and having a plurality of triangular-shaped teeth extending peripherally, and at least one wheel stopper mounted to the chassis and being engageable with a respective one of the at least one gear disk and being configurable in a displacement configuration wherein the at least one wheel stopper is disengaged from the respective one of the at least one gear disk to allow rotation of the respective one of the wheels and a braking configuration wherein the at least one wheel stopper is engaged with the respective one of the gear disk to prevent rotation of the at least one of the wheels.

In an embodiment, the gear disk includes a plurality of rounded recesses with adjacent ones of the triangular-shaped teeth being separated by a respective one of the rounded recesses.

In an embodiment, the wheel stopper comprises a tooth engaging brake lever pivotally mounted to the chassis and having at least one round-ended tooth. The at least one round-ended tooth can comprise at least a pair of round-ended teeth having a pointed tooth receiving recess defined in between. The round-ended teeth of the tooth engaging brake lever can be substantially complementary in shape with the triangular-shaped teeth of the at least one gear disk.

In an embodiment, the rollator can further comprise a wheel braking system comprising at least two of the wheel braking assembly and a seat assembly including a seat pivotally mounted to the chassis and selectively configurable in a raised configuration and a seated configuration wherein the seat extends substantially horizontally in the seat/walker receiving area, the seat assembly being operatively connected to the wheel braking system, wherein, when the seat is configured in the seated configuration, the wheel braking assemblies are configured in the displacement configuration if a pressure equal to or greater than a seat pressure threshold is applied thereon, and are configured in the braking configuration otherwise.

In an embodiment, the rollator can further comprises two handgrip assemblies, each one including a pivotable handgrip operatively connected to the wheel braking system, the handgrips being selectively configurable in a handgrip displacement configuration and in a handgrip braking configuration, and wherein the wheel braking system is configured in the braking configuration in the raised configuration of the seat unless the two handgrips are configured in the handgrip displacement configuration. Each one of the handgrips can selectively configurable in a raised configuration and a lowered configuration. If the seat is configured in the raised configuration, configuration of at least one of the handgrips in the raised configuration can configure the wheel braking system in the braking configuration and configuration of at least one of the handgrips in the lowered configuration with a pressure applied thereon equal to or greater than the handgrip pressure threshold can configure the wheel braking system in the braking configuration; and configuration of both of the handgrips in the lowered configuration with a pressure applied thereon being lowered than the handgrip pressure threshold configures the wheel braking system in the displacement configuration. The handgrips can be configured in the raised configuration when no pressure is applied thereon. The handgrips can be configured in the raised configuration when no pressure is applied thereon.

In an embodiment, the at least one pair of wheels comprises a pair of fixed wheels mounted to rear ends of the lateral side portions of the chassis and the rollator further comprises swivel wheels rotatably mounted to the front portion of the chassis.

In an embodiment, the seat/walker receiving area is unobstructed when the seat is configured in the raised configuration.

In an embodiment, the lateral side portions are pivotally mounted to the respective end of the front portion and selectively configurable in an operative configuration wherein they are spaced-apart from one another to define the seat/walker receiving area inbetween and a compacted configuration wherein they are superposed to each other and extend substantially parallel to the front portion. The seat can be configured in the raised configuration in the compacted configuration of the lateral side portions and can be located between the front portion and the lateral side portions of the chassis.

According to another general aspect, there is provided a rollator for mobility assistance to a user. The rollator comprises:
a chassis including:
   a front portion and two lateral side portions, each one of the lateral side portions extending rearwardly from a respective end of the front portion;
at least one right-side wheel and at least one left-side wheel rotatably mounted to at least one of the front portion of the chassis and rear ends of the lateral side portions of the chassis;
at least a right-side and a left-side wheel braking assemblies, each one of the wheel braking assemblies having a wheel stopper engageable with a respective one of the right-side and left-side wheels and configurable in a displacement configuration allowing rotation of the respective one of the right-side and left-side wheels and a braking configuration wherein the wheel stopper is engaged with the respective one of the right-side and left-side wheels and prevent rotation thereof; and
a pair of pivotable handgrips operatively connected to the wheel braking assemblies and being selectively configurable in a handgrip displacement configuration and in a handgrip braking configuration, wherein configuration of at least one of the handgrips in the handgrip braking configuration configures the wheel stoppers in the braking configuration wherein they are engaged with their respective one of the right-side and left-side wheels.

In an embodiment, the at least one right-side wheel and at least one left-side wheel comprises a right-side fixed wheel and a left-side fixed wheel and the wheel stopper of the at least right-side wheel braking assembly is engageable with the right-side fixed wheel and the wheel stopper of the at least left-side wheel braking assembly is engageable with the left-side fixed wheel. The right-side and left-side fixed wheels can be rotatably mounted to a respective one of the rear ends of the lateral side portions of the chassis and the rollator can further comprise swivel wheels rotatably mounted to the front portion of the chassis.

In an embodiment, the right-side and the left-side wheel braking assemblies can be configured in the displacement configuration if both handgrips are configured in the handgrip displacement configuration.

In an embodiment, the chassis defined by the front portion and the two lateral side portions has a substantially U-shape profile with the front and lateral side portions defining a seat/walker receiving area inbetween; and the rollator further comprises a seat assembly including a seat pivotally mounted to the chassis and selectively configurable in a raised configuration and a seated configuration wherein the seat extends substantially horizontally in the seat/walker receiving area, the seat/walker receiving area being unobstructed when the seat is configured in the raised configuration. The lateral side portions can be pivotally mounted to the respective end of the front portion and selectively configurable in an operative configuration wherein they are spaced-apart from one another to define the seat/walker receiving area inbetween and a compacted configuration wherein they are superposed to each other and extend substantially parallel to the front portion. The seat can be configured in the raised configuration in the compacted configuration of the lateral side portions and can be located between the front portion and the lateral side portions of the chassis.

In an embodiment, the handgrips are pivotable and:
the handgrip displacement configuration comprises a lowered configuration in which the handgrips extend substantially horizontally and wherein a pressure below a handgrip pressure threshold is applied thereon, and wherein the wheel braking assemblies are disengaged from their respective wheels if both handgrips are simultaneously configured in the handgrip displacement configuration; and
   the handgrip braking configuration comprises:
      a raised configuration of the handgrips wherein the handgrips extend above the handgrip displacement configuration and a lowerable into the handgrip displacement configuration; and
      a excessive pressure configuration wherein the handgrips are configured in the lowered configuration and a pressure equal to or greater than the handgrip pressure threshold is applied thereon.

In an embodiment, the handgrips are configured in the raised configuration when no pressure is applied thereon.

In an embodiment, the rollator further comprises a wheel braking system comprising the wheel braking assemblies which is operatively connected to the seat of the seat assembly. When the seat is configured in the seated configuration with a pressure greater than or equal to a seat pressure threshold applied thereon, the wheel braking assemblies can be configured in the displacement configuration and when the seat is configured in the seated configuration with a pressure lower than the seat pressure threshold applied thereon, the wheel braking assemblies can be configured in the braking configuration. The wheel braking system can further comprise a wheel braking assembly controller mounted to the chassis and operatively connecting the handgrips to the wheel braking assemblies. In an embodiment, the wheel braking assembly controller comprises:
a pair of outer tubes, each one of the outer tubes being operatively connected to a respective one of the handgrips and being rotatable along a rotation axis thereof upon actuation of the respective one of the handgrips;
a shaft operatively connected to the wheel braking assemblies, the shaft having a substantially cylindrical portion at least partially enclosed into the outer tubes and a joint protruding from the cylindrical portion, the shaft being rotatable along a rotation axis thereof to configure the wheel braking assemblies simultaneously in one of the braking configuration and the displacement configuration, wherein the outer tubes abut against the joint when rotating simultaneously to engage the shaft in rotation and wherein the shaft is not engaged in rotation when only one of the outer tubes is engaged in rotation.

In an embodiment, the joint is bow shaped and the outer tubes comprises an indentation to receive a portion of the bow shaped joint therein.

In an embodiment, the wheel braking assembly controller comprises: a power supply, handgrip sensors operatively connected to the handgrips, a logic controller, electric connectors connecting the handgrip sensors to the wheel braking systems through the logic controller, and a power supply operatively connected to at least one of the handgrip sensors, the logic controller and the wheel braking assemblies to supply electric power thereto. The handgrip sensors can comprise limit switches detecting a configuration of a respective one of the handgrips.

In an embodiment, the rollator further comprises a handgrip assembly comprising at least one handgrip frame pivotally mounted to the chassis and including lateral side portions extending substantially in a same plane than a respective one of the lateral side portions of the chassis, each one of the lateral side portions of the at least one handgrip frame having one of the pair of handgrips pivotally mounted thereto, the at least one handgrip frame being selectively configurable in an operative configuration wherein at least one of the handgrips is located rearwardly of the front portion of the chassis and a frontward configuration wherein at least one of the handgrips is located forwardly of the front portion of the chassis.

In an embodiment, each one of the wheel braking assemblies comprises a gear disk secured to the respective one of the wheels, the gear disk having a plurality of triangular-shaped teeth extending peripherally and the respective one of the wheel stoppers is engageable with the respective gear disk in the braking configuration to prevent rotation of the respective one of the wheels. Each one of the gear disks can include a plurality of rounded recesses, adjacent ones of the triangular-shaped teeth being separated by one of the rounded recesses defined inbetween. Each one of the wheel stoppers can comprise a tooth engaging brake lever pivotally mounted to the chassis and having at least one round-ended tooth. The at least one round-ended tooth can comprise at least a pair of round-ended teeth, each one of round-ended teeth being spaced-apart by a pointed tooth receiving recess. The round-ended teeth of the tooth engaging brake lever can be substantially complementary in shape with the triangular-shaped teeth of the gear disk.

In an embodiment, the chassis further comprises at least one accessory receiving tube extending substantially vertically.

In an embodiment, the rollator further comprises a walking re-education assembly including a frame removably engageable with the chassis and a ring, the frame having two lateral side portions spaced-apart from one another and extending substantially parallel to a respective one of the lateral side portions of the chassis when engaged therewith, the ring being attached to the two lateral side portions of the frame and extending between, the ring being configured to partially support a weight of the user during a rehabilitation process. The walking re-education assembly can further comprise at least a pair of rear wheels rotatably mounted to a respective one of the lateral side portions of the frame.

In an embodiment, the fixed wheels comprise at least two sets of fixed wheels, each one of the sets of fixed wheels including at least one left-side fixed wheel and one right-side fixed wheel and each one of the sets being characterized by a different wheel diameter, the fixed wheels being detachably, selectively and rotatably securable to the lateral side portions of the chassis.

According to another general aspect, there is provided a rollator for mobility assistance to a user. The rollator comprises:
a chassis;
at least one pair of wheels rotatably mounted to the chassis;
wheel braking assemblies engageable with the at least one pair of wheels and configurable in a displacement configuration allowing rotation of the wheels and a braking configuration wherein a respective one of the wheel braking assemblies is engaged with a respective one of the wheels to prevent rotation thereof; and
handgrip assemblies including pivotable handgrips operatively connected to at least one of the wheel braking assemblies, each one of the handgrips being selectively configurable in:
   a raised configuration wherein the at least one of the wheel braking assemblies is engaged with at least one of the wheels to prevent rotation thereof; and
   a lowered configuration corresponding to a walking configuration of the rollator, and if pressure applied on at least one of the handgrips is equal to or above a handgrip pressure threshold, at least one of the wheel braking assemblies is engaged with a respective one of the wheels to prevent rotation thereof.

In an embodiment, the handgrip is configured in the raised configuration when no pressure is applied thereon.

In an embodiment, the wheel braking assemblies are configured in the displacement configuration if both handgrips are configured in the lowered configuration and if the pressure applied on both handgrips is below the handgrip pressure threshold.

In an embodiment, each one of the handgrip assemblies comprises a biasing member biasing a respective one of the handgrips into the raised configuration.

In an embodiment, the handgrip assemblies comprise two handgrip assemblies and the wheel braking assemblies are configured in the braking configuration if at least one of the two handgrips is configured in one of the raised configuration and the lowered configuration with a pressure applied thereon greater than or equal to the handgrip pressure threshold.

In an embodiment, the handgrip assemblies comprise two handgrip assemblies and the wheel braking system is configured in the displacement configuration if the two handgrips are configured in the lowered configuration with a pressure applied thereon lower than the handgrip pressure threshold.

In an embodiment, the chassis includes a front portion and two lateral side portions, each one of the lateral side portions extending rearwardly from a respective end of the front portion, and the at least one pair of wheels comprises a pair of fixed wheels mounted to rear ends of the lateral side portions of the chassis and the rollator further comprises swivel wheels rotatably mounted to the front portion of the chassis. The chassis defined by the front portion and the two lateral side portions can have a substantially U-shape profile with the front and lateral side portions defining a seat/walker receiving area inbetween. The rollator can further comprise a seat assembly including a seat pivotally mounted to the chassis and selectively configurable in a raised configuration and a seated configuration wherein the seat extends substantially horizontally in the seat/walker receiving area, the seat/walker receiving area being unobstructed when the seat is configured in the raised configuration. The lateral side portions can be pivotally mounted to the respective end of the front portion and selectively configurable in an operative configuration wherein they are spaced-apart from one another to define the seat/walker receiving area inbetween and a compacted configuration wherein they are superposed to each other and extend substantially parallel to the front portion. The seat can be configured in the raised configuration in the compacted configuration of the lateral side portions and can be located between the front portion and the lateral side portions of the chassis.

In an embodiment, the wheel braking system is operatively connected to the seat of the seat assembly, wherein when the seat is configured in the seated configuration with a pressure greater than or equal to a seat pressure threshold applied thereon, the wheel braking system is configured in the displacement configuration and when the seat is configured in the seated configuration with a pressure lower than the seat pressure threshold applied thereon, the wheel braking system is configured in the braking configuration. The handgrip assemblies can comprise two handgrip assemblies and the wheel braking system can further comprise a wheel braking assembly controller mounted to the chassis. The wheel braking assemblies can have wheel stoppers engageable with a respective one of the wheels, each one of the wheel stoppers being selectively configurable in the displacement configuration wherein it is disengaged from the respective one of the wheels to allow rotation thereof and in the braking configuration wherein it is engaged with the respective one of the wheels to prevent rotation thereof. The wheel braking assembly controller can operatively connect the handgrips to the wheel braking assemblies. The wheel braking assembly controller can comprise:
a pair of outer tubes, each one of the outer tubes being operatively connected to a respective one of the handgrips and being rotatable along a rotation axis thereof upon actuation of the respective one of the handgrips;
a shaft operatively connected to the wheel braking assemblies, the shaft having a substantially cylindrical portion at least partially enclosed into the outer tubes and a joint protruding from the cylindrical portion, the shaft being rotatable along a rotation axis thereof to configure the wheel braking assemblies simultaneously in one of the braking configuration and the displacement configuration, wherein the outer tubes abut against the joint when rotating simultaneously to engage the shaft in rotation and wherein the shaft is not engaged in rotation when only one of the outer tubes is engaged in rotation.

In an embodiment, the joint is bow shaped and each one of the outer tubes comprises an indentation to receive a portion of the bow shaped joint therein. The wheel braking assembly controller can comprise: a power supply, handgrip sensors operatively connected to the handgrips, a logic controller, electric connectors connecting the handgrip sensors to the wheel braking assemblies through the logic controller, and a power supply operatively connected to at least one of the handgrip sensors, the logic controller and the wheel braking assemblies to supply electric power thereto. The handgrip sensors can comprise limit switches detecting a configuration of a respective one of the handgrips.

In an embodiment, the wheel braking system comprises a gear disk secured to the respective one of the wheels, the gear disk having a plurality of triangular-shaped teeth extending peripherally and the respective one of the wheel stoppers is engageable with the respective gear disk in the braking configuration to prevent rotation of the respective one of the wheels. Each one of the gear disks can include a plurality of rounded recesses, adjacent ones of the triangular-shaped teeth being separated by one of the rounded recesses defined inbetween. Each one of the wheel stoppers can comprise a tooth engaging brake lever pivotally mounted to the chassis and having at least one round-ended tooth. The at least one round-ended tooth can comprise at least a pair of round-ended teeth, each one of the round-ended teeth being spaced-apart by a pointed tooth receiving recess. The round-ended teeth of the tooth engaging brake lever can be substantially complementary in shape with the triangular-shaped teeth of the gear disk.

In an embodiment, the handgrip assemblies comprises at least one handgrip frame pivotally mounted to the chassis and having the handgrip pivotally mounted thereto, the at least one handgrip frame being selectively configurable in an operative configuration wherein the handgrip is located rearwardly of the chassis and a frontward configuration wherein the handgrip is located forwardly of the chassis.

In an embodiment, the chassis further comprises at least one accessory receiving tube extending substantially vertically.

In an embodiment, the rollator further comprises a walking re-education assembly including a frame removably engageable with the chassis and a ring, the frame having two lateral side portions spaced-apart from one another and extending substantially parallel to a respective one of the lateral side portions of the chassis when engaged therewith, the ring being attached to the two lateral side portions of the frame and extending between, the ring being configured to partially support a weight of the user during a rehabilitation process. The walking re-education assembly can further comprise at least a pair of rear wheels rotatably mounted to a respective one of the lateral side portions of the frame.

In an embodiment, the fixed wheels comprise at least two sets of fixed wheels, each one of the sets of fixed wheels including at least one left-side fixed wheel and one right-side fixed wheel and each one of the sets being characterized by a different wheel diameter, the fixed wheels being detachably, selectively and rotatably securable to the lateral side portions of the chassis.

According to another general aspect, there is provided a rollator for mobility assistance to a user. The rollator comprises:
a chassis including:
   a front portion and two lateral side portions, each one of the lateral side portions extending rearwardly from a respective end of the front portion to define a seat/walker receiving area;
at least one pair of wheels rotatably mounted to the chassis;
a wheel braking system engageable with the at least one pair of wheels and configurable in a displacement configuration allowing rotation of the wheels and a braking configuration wherein the wheel braking system is engaged with at least one of the at least one pair of wheels to prevent rotation thereof; and
a seat assembly including a seat pivotally mounted to the chassis and selectively configurable in a raised configuration and a seated configuration wherein the seat extends substantially horizontally in the seat/walker receiving area, the seat assembly being operatively connected to the wheel braking system, wherein, when the seat is configured in the seated configuration, the wheel braking system is configured in the displacement configuration.

In an embodiment, when the seat is configured in the seated configuration, the wheel braking system is configured in the displacement configuration if a pressure greater than or equal to a seat pressure threshold is applied thereon, and is configured in the braking configuration otherwise. The seat can be further pivoted downwardly towards a horizontally-extending configuration when a pressure equal to or greater than the seat pressure threshold is applied thereto.

The rollator can further comprise two handgrip assemblies, each one including a pivotable handgrip operatively connected to the wheel braking system, the handgrips being selectively configurable in a handgrip displacement configuration and in a handgrip braking configuration, and wherein the wheel braking system is configured in the braking configuration in the raised configuration of the seat unless the two handgrips are configured in the handgrip displacement configuration. In an embodiment, each one of the handgrips is selectively configurable in:
a raised configuration and a lowered configuration, wherein, if the seat is configured in the raised configuration, configuration of at least one of the handgrips in the raised configuration configures the wheel braking system in the braking configuration and configuration of at least one of the handgrips in the lowered configuration with a pressure applied thereon equal to or greater than a handgrip pressure threshold configures the wheel braking system in the braking configuration; and configuration of both of the handgrips in the lowered configuration with a pressure applied thereon being lowered than the handgrip pressure threshold configures the wheel braking system in the displacement configuration.

The handgrips can be configured in the raised configuration when no pressure is applied thereon.

In an embodiment, the at least one pair of wheels comprises a pair of fixed wheels mounted to rear ends of the lateral side portions of the chassis and the rollator further comprises swivel wheels rotatably mounted to the front portion of the chassis.

In an embodiment, the seat/walker receiving area is unobstructed when the seat is configured in the raised configuration.

In an embodiment, the lateral side portions are pivotally mounted to the respective end of the front portion and selectively configurable in an operative configuration wherein they are spaced-apart from one another to define the seat/walker receiving area inbetween and a compacted configuration wherein they are superposed to each other and extend substantially parallel to the front portion. The seat can be configured in the raised configuration in the compacted configuration of the lateral side portions and can be located between the front portion and the lateral side portions of the chassis.

The wheel braking system can further comprise a wheel braking assembly controller mounted to the chassis and wheel braking assemblies having wheel stoppers engageable with a respective one of the wheels. Each one of the wheel stoppers can be selectively configurable in the displacement configuration wherein it is disengaged from the respective one of the wheels to allow rotation thereof and in the braking configuration wherein it is engaged with the respective one of the wheels to prevent rotation thereof. The wheel braking assembly controller can operatively connect the handgrips to the wheel braking assemblies.

In an embodiment, the two handgrip assemblies comprise at least one handgrip frame pivotally mounted to the chassis and having the handgrips pivotally mounted thereto, the at least one handgrip frame being selectively configurable in an operative configuration wherein at least one of the handgrips is located rearwardly of the front portion of the chassis and a frontward configuration wherein at least one of the handgrips is located forwardly of the front portion of the chassis.

In an embodiment, the rollator further comprise a walking re-education assembly including a frame removably engageable with the chassis and a ring, the frame having two lateral side portions spaced-apart from one another and extending substantially parallel to a respective one of the lateral side portions of the chassis when engaged therewith, the ring being attached to the two lateral side portions of the frame and extending between, the ring being configured to partially support a weight of the user during a rehabilitation process. The fixed wheels can comprise at least two sets of fixed wheels, each one of the sets of fixed wheels including at least one left-side fixed wheel and one right-side fixed wheel and each one of the sets being characterized by a different wheel diameter, the fixed wheels being detachably, selectively and rotatably securable to the lateral side portions of the chassis.

According to still another general aspect, there is provided a rollator for mobility assistance to a user. The rollator comprises:
a chassis including:
   a front portion and two lateral side portions, each one of the lateral side portions extending rearwardly from a respective end of the front portion to define a seat/walker receiving area;
at least one pair of wheels rotatably mounted to the chassis;
a wheel braking system engageable with the at least one pair of wheels and configurable in a displacement configuration allowing rotation of the wheels and a braking configuration wherein the wheel braking system is engaged with at least one of the at least one pair of wheels to prevent rotation thereof; and
a seat assembly including a seat pivotally mounted to the chassis and selectively configurable in a raised configuration, a first seated configuration, and a second seated configuration, wherein, in the first and second seated configurations, the seat extends substantially horizontally in the seat/walker receiving area and the second seated configuration is reached when a pressure equal to or greater than a seat pressure threshold is applied on the seat configured in the first seated configuration, the seat assembly being operatively connected to the wheel braking system, wherein when the seat is configured in the second seated configuration, the wheel braking system is configured in the displacement configuration and, when the seat is configured in the first seated configuration, the wheel braking system is configured in the braking configuration.

In an embodiment, an angle between about 2.5° and about 5° is defined between the first and the second seated configurations with the seat being pivoted further downwardly in the second seated configuration.

According to another general aspect, there is provided a rollator for mobility assistance to a user. The rollator comprises:
a chassis including:
   a front portion, two lateral side portions, and at least one accessory receiving tube attached to at least one of the lateral side portions and front portion; and
at least one pair of wheels rotatably mounted to the chassis.

In an embodiment, the at least one accessory receiving tube extends substantially vertically at a rear end of a respective one of the lateral side portions.

According to still another general aspect, there is provided a rollator for mobility assistance to a user. The rollator comprises:
a chassis including:
   a front portion and two lateral side portions, each one of the lateral side portions extending rearwardly from a respective end of the front portion;
a pair of front wheels rotatably mounted to the front portion of the chassis;
a pair of rear wheels rotatably mounted to a respective one of the lateral side portions of the chassis; and
a walking re-education assembly comprising a frame removably engageable with the chassis and a ring, the frame having two lateral side portions spaced-apart from one another and extending substantially parallel to a respective one of the lateral side portions of the chassis when engaged therewith, the ring being attached to the two lateral side portions of the frame and extending between, the ring being configured to partially support a weight of the user during a rehabilitation process.

In an embodiment, the walking re-education assembly further comprises at least a pair of rear wheels rotatably mounted to a respective one of the lateral side portions of the frame.

According to still another general aspect, there is provided a method for re-educating walking capacities for a person having motory deficiencies using the rollator as described above.

According to a further general aspect, there is provided a rollator for mobility assistance to a user. The rollator comprises:
a chassis including: a front portion and two lateral side portions, each one of the lateral side portions extending rearwardly from a respective end of the front portion;
a pair of front wheels rotatably mounted to the front portion of the chassis; and
at least two sets of rear wheels, each one of the sets of rear wheels including at least a pair of rear wheels and being characterized by a different wheel diameter, the rear wheels being detachably and rotatably securable to the lateral side portions of the chassis and each one of the sets being selectively securable to the lateral side portions of the chassis.

In an embodiment, the lateral side portions of the chassis comprise at least two sets of interchangeable downwardly extending support members and each one of the sets of interchangeable downwardly extending support members is characterized by a different length and corresponds to one of the sets of rear wheels.

According to another general aspect, there is provided a kit to assemble a rollator for mobility assistance to a user. The kit comprises:
a substantially U-shaped chassis including:
   a front portion and two lateral side portions, each one of the lateral side portions being securable to the front portion at a respective end thereof to extend therefrom to define a seat/walker receiving area;
at least one pair of wheels rotatably engageable the chassis; and
a seat assembly including a seat frame pivotally engageable with the chassis and a seat pivotally engageable with the seat frame and, when the seat assembly is engaged with the chassis, the seat being selectively configurable in a raised configuration and a seated configuration wherein the seat extends substantially horizontally in the seat/walker receiving area, the seat/walker receiving area being unobstructed when the seat is configured in the raised configuration.

In an embodiment, the kit further comprises a wheel braking system securable to the chassis and engageable with the at least one pair of wheels and, when the wheel braking system is engaged with the at least one pair of wheels, configurable in a displacement configuration allowing rotation of the wheels and a braking configuration wherein the wheel braking system is engaged with at least one of the at least one pair of wheels to prevent rotation thereof.

In an embodiment, the kit further comprises two handgrip assemblies, each one including a pivotable handgrip, handgrip control cables operatively connectable to the wheel braking system, and, wherein when the handgrip assemblies are mounted to the chassis, the handgrips being selectively configurable in a handgrip displacement configuration and in a handgrip braking configuration.

In an embodiment, the at least one pair of wheels comprises a pair of fixed wheels rotatably securable to rear ends of the lateral side portions of the chassis and the rollator further comprises swivel wheels rotatably securable to the front portion of the chassis.

In an embodiment, the lateral side portions are pivotally engagable to the respective end of the front portion and, when the lateral side portions are pivotally mounted to the front portion, they are selectively configurable in an operative configuration wherein they are spaced-apart from one another to define the seat/walker receiving area inbetween and a compacted configuration wherein they are superposed to each other and extend substantially parallel to the front portion. The seat can be mounted to the chassis. It can be configured in the raised configuration in the compacted configuration of the lateral side portions and can be located between the front portion and the lateral side portions of the chassis.

In an embodiment, the wheel braking system further comprises a wheel braking assembly controller securable to the chassis and wheel braking assemblies having wheel stoppers engageable with a respective one of the wheels, and, wherein, the wheel braking assemblies are mounted to the chassis, each one of the wheel stoppers being selectively configurable in the displacement configuration wherein it is disengaged from the respective one of the wheels to allow rotation thereof and in the braking configuration wherein it is engaged with the respective one of the wheels to prevent rotation thereof; and wherein the wheel braking assembly controller is operatively connectable the handgrips to the wheel braking assemblies. In an embodiment, the two handgrip assemblies can comprise at least one handgrip frame pivotally securable to the chassis and having the handgrips pivotally securable thereto, and, when mounted to the chassis, the at least one handgrip frame being selectively configurable in an operative configuration wherein at least one of the handgrips is located rearwardly of the front portion of the chassis and a frontward configuration wherein at least one of the handgrip is located forwardly of the front portion of the chassis.

In an embodiment, the kit further comprises a walking re-education assembly including a frame removably engageable with the chassis and a ring, the frame having two lateral side portions spaced-apart from one another and extending substantially parallel to a respective one of the lateral side portions of the chassis when engaged therewith, the ring being attachable to the two lateral side portions of the frame and extending between, the ring being configured to partially support a weight of the user during a rehabilitation process. In an embodiment, the fixed wheels comprise at least two sets of fixed wheels, each one of the sets of fixed wheels including at least one left-side fixed wheel and one right-side fixed wheel and each one of the sets being characterized by a different wheel diameter, the fixed wheels being detachably, selectively and rotatably securable to the lateral side portions of the chassis.

In an embodiment, the wheel braking system further comprises gear disks, each one being securable to the respective one of the wheels and having a plurality of triangular-shaped teeth extending peripherally and the respective one of the wheel stoppers is engageable with the respective one of the gear disks in the braking configuration to prevent rotation of the respective one of the wheels.

In an embodiment, the kit further comprises at least one accessory receiving tube securable to the chassis and extending substantially vertically when secured thereto.

According to a general aspect, there is provided a multifunctional rollator having a chassis selectively configurable in an operative configuration and in a compacted configuration that may be used as a walker, as a transport chair, as a wheelchair, or as an office wheeled chair. In some embodiments, the rollator may be provided with a fall detection capability and/or a re-educational or re-habilitation capability, i.e. a walking re-education assembly, to be exploited by a professional or non-professional user. In the compacted (or folded), the rollator is compacted in a “easy-to-store” geometry.

In an embodiment, the rollator is provided with a wheel braking system, including wheel braking assemblies, that may reassure a user that in any time the wheel braking assemblies are engaged with at least some of the wheels of the rollator unless forces are applied correctly on handgrips and/or if the user is safely seated on the rollator seat. In an embodiment, each one of the wheel braking assemblies includes a gear disk that may allow a precise and efficient braking that may contribute to minimizing the delay before brake activation. In an embodiment, the gear disk has a plurality of triangular-shaped teeth extending peripherally and each one of the wheel braking assemblies further includes a wheel stopper having recesses complementary in shape to the triangular-shaped teeth that may reduce the hovering effect of the wheel stopper teeth in comparison with other gear braking systems. Furthermore, it may reduce the delay before the wheel braking system components engage with each other.

In an embodiment, there is provided an automatic braking system, that can be disengaged from the wheels when both hands are in contact with handgrips. This automatic braking system can represent an educative functionality helping the health professional to teach the right rollator use.

In an embodiment, there is provided a rollator that may show increased stability, especially when body weight of a user is being transferred to the chassis. This feature may reduce the potential overthrow of the multifunctional rollator.

In an embodiment, there is provided a set of secondary handgrips, engageable with the rollator chassis. The secondary handgrips can act as lateral support. They can be finely adjustable to be used from a seated position to a standing position or from the standing position to the seated position.

In an embodiment, the seat assembly is provided a braking functionality wherein the seat is operatively connected to the wheel braking system. More particularly, the wheel braking system is engaged with the wheels unless the user is seated and safe in which condition and the wheel braking system is disengaged with the wheels. This functionality acting as an integrated anti-roll back system.

In an embodiment, the rollator is provided a chassis that allows multi-adjustability wherein the multifunctional rollator can be precisely adapted to the morphology of the user, independently from right to left.

In an embodiment, the rollator is provided a seat assembly and handgrips for which a health professional can adjust the pressure or weight required to disengage the wheel braking system, or in other words, adjusting the fall detection system to fit the user's behavior, weight or morphology.

In an embodiment, the rollator shows a reduced ground footprint which allows a reduced space congestion, and provides an improved field of view forwardly which allows a better visual detection of obstacles in front of the multifunctional rollator.

In an embodiment, the rollator offers a frontal support while the user is standing up working on a counter height surface of work. In an embodiment, the rollator is adapted to be functional in a non-adapted environment, particularly when used in a non-adapted toilet stall.

In an embodiment, the seat is pivotally mounted to the chassis and is foldable in a raised configuration that may provide an improved field of view when using the multifunctional rollator.

In an embodiment, the primary handgrips are pivotally mounted to the chassis and are thus capable of being toggled away for greater comfort and additional functionality when the multifunctional rollator is being used in its transport chair mode, its office wheeled chair mode or in its wheelchair configuration.

In an embodiment, the anti-roll back system of the rollator is operatively connected to the seat and is integrated into the chassis. Therefore, it may not show the habitual discomfort related to a detection system that would be installed underneath the seat.

In an embodiment, the rollator is relatively lightweight and compact, when configured in the compacted configuration, i.e. folded.

In an embodiment, an operating pressure for the seat and the primary handgrips, i.e. the seat pressure threshold and the handgrip pressure threshold, is fully adjustable to adapt to the behavior of any given user.

In an embodiment, the set(s) of handgrips, primary and/or secondary, have height that may be finely adjusted through a scaled indicator giving precise reading and data to the health professional.

In an embodiment, the rollator is provided with a hazard detection system, i.e. a brake assembly controller, that interprets the user's hands behavior and determines whether or not the user is in a safe set of conditions (logical interpretation of hands behavior).

In an embodiment, the shape of the chassis may provide to the user a better lateral and frontal stability giving the fact that the user is travelling inside the chassis of the multifunctional rollator. In an embodiment, the rollator is provided with a relatively centered center of gravity, especially linked with a relative position of the user inside the chassis, which results in reducing the potential overthrow of the multifunctional rollator, when for example excessive pressure is applied which characterized the behavior of a user falling.

In an embodiment, there is provided a rollator configured so that a user is positioned inside the chassis making the user and the multifunctional rollator combination a more compact combination thus reducing space congestion in public area. In an embodiment, the user is positioned inside the chassis when walking with the rollator, rendering a combination of the user and the multifunctional rollator more compact combination and, consequently, reducing space congestion in public area.

In an embodiment, there is provided a rollator that may allow an optimal arm to body angle (via adjustable features), which permits the user a higher and better weight transfer to the multifunctional rollator, thus preventing overthrow and helping in the re-education process.

In an embodiment, due to the chassis configuration, the caregiver can be provided with enough space to push the multifunctional rollator when used in the transport chair mode or with a wheelchair configuration, comparable to the space available with the use of a conventional wheelchair.

In an embodiment, the user may be provided with enough feet space when the multifunctional rollator is used in the transport chair mode. The rollator can be provided with an alternative propulsion mode when the multifunctional rollator is used into its wheelchair configuration (push rims). In an embodiment, the multifunctional rollator can be used with an alternative retractable lever (paddling type of propulsion).

In an embodiment, the rollator can include a backrest adjustable in height and in angle or adjustable declination (relative to the rest seat).

In an embodiment, the rollator can include one or more accessory receiving tubes to integrate many useful accessories without any technical assistance. For instance, the accessories that can be removably engaged with the accessory receiving tubes can be any accessory that may improve the comfort of the user comprising, such as and without being limitative, wheelchair type foldable feet rest supports. The feet rest supports can be foldable so that the user may propel himself with his feet when the rollator is used in the transport chair mode or in the wheelchair mode. The accessories engageable with the rollator accessory receiving tubes can also include accessories relative to physical or medical condition of the user such as an oxygen tank support, a solute pole, or a medical monitor, and the like.

In an embodiment, a walking re-education system can be mounted to the rollator chassis. The walking re-education system can include a physiotherapy rear extension with a twist belt that supports the weight of the user while being re-educated. Given the fact that in rehabilitation the number of training hours reduces the time for rehabilitation, that falls occur mainly at the beginning of the rehabilitation process, that more and more persons will require rehabilitation, that physiotherapist needs will increase in the near future and that the physiotherapist availability will be reduced, this equipment may be appreciated. The rollator may offer an advantageous alternative to the existing rehabilitation solutions that are most of the time heavy, not well adapted and cumbersome. In some implementations, it may be used with minimal supervision from a physiotherapist, given the safety functionality of the physiotherapy rear extension. The combination of the physiotherapy rear extension with the twist belt and the weight support device may be adjusted to support between 20 to 100% of the user's body weight, which may help in progressively reloading the user's legs.

Other accessories engageable with the rollator accessory receiving tubes can also include a cup holder, a telephone holder, an electronic tablet holder or lateral storage bags that can be quickly installed without tools or technical assistance.

In an embodiment, the shape and configuration of the chassis allows the use of the rollator in public adapted or non-adapted public toilets or private toilet stalls. More particularly, the ground to front portion of the rollator clearance that allows the multifunctional rollator to be rolled over a regular toilet bowl. The chassis is configured to allow the user to use the secondary handgrips or to hold while sitting down on the toilet seat so that the multifunctional rollator is no longer disturbing the area surrounding the toilet and becomes a tool that allows the user to use non-adapted toilet stalls.

In an embodiment, the secondary handgrips can be adjusted in height according to the user morphology in order to have an adapted height to sit on the toilet seat.

According to a general aspect, there is provided a multifunctional rollator allowing a user to move himself in an autonomous way, or semi-autonomously way and to use the non-adapted daily life commodities keeping acceptable ergonomic positioning while the user positions himself in a cleared space inside the rollator U-shaped chassis of the multifunctional rollator. In an embodiment, the multifunctional rollator allows the user to move himself in an autonomous and safe way in a seated or walking position while having access to non-adapted daily life commodities, the multifunctional rollator being equipped with four (4) vertical members extending towards the ground and having at least one wheel on each extension and being equipped with at least one of the following devices:
a wheel braking assembly comprising wheel stoppers allowing to engage with or disengage from at least one of the wheels under controlled pressure or weight and/or in case a fall occurs;
a seat pivotally mounted to the chassis having at least two configurations: a first position, i.e. the seated configuration or horizontal position where the user can sit down in case of need, and a second position, i.e. the raised configuration or vertical position where the interior of the U-shaped chassis is cleared and where the user can position himself to walk around assisted by the multifunctional rollator realizing his daily tasks;
a set of secondary handgrips on which the user can take hold while standing up or sitting down; and
a second set of propulsion (rear) wheels including push rims. The second set of rear wheels can be mounted to the chassis, laterally on each rear side of the multifunctional rollator. The propulsion wheels can be used to ease the displacement of the multifunctional rollator through the push rims coaxially attached to the wheels.

In an embodiment, the multifunctional rollator also includes:
a front portion and two lateral side portions positioned sensibly at about right angle with the frontal portion to define a “U” shaped cleared and empty area inbetween;
a mobility system (supporting elements or chassis of the multifunctional rollator in contact with the ground offering reduced friction and increased support and stability) including, preferably four (4) wheels in contact with the ground in which:
   Two (2) wheels are positioned closed to the front portion and rotatably mounted to a lower end of the chassis, and
   Two (2) wheels positioned closed to a rear end of the chassis, wherein each wheel is rotatably mounted to a lower end of each one of the lateral side portions;
at least two (2) support handgrips (secondary handgrips), which can be adjustable in height, engaged about symmetrically to a respective one of the lateral side portions of the chassis;
a foldable rest seat, foldable against a backrest, foldable either against a lateral side portions of the chassis (either left or right); and
a blocking/de-blocking device comprising two (2) handgrips (i.e. primary handgrips) which can be mounted to two swing arms and are configured to control the engagement and the disengagement of the wheel braking assemblies from the wheels. Each one of the handgrips is operatively connected to a wheel braking system which controls a rotation of at least one of the wheels. In an embodiment, the wheel braking system is disengaged only if both primary handgrips are in the disengaged position. On the contrary, the wheel braking system is engaged if only one of the handgrips is in the disengaged position.

In an embodiment, the multifunctional rollator also includes a fall detection/braking system, i.e. wheel braking assembly controller, which is configured to realize at least one of the following functions:
the configuration of at least one wheel, preferably at least two wheels, in the wheel braking configuration when only one of the primary handgrip is lowered in a displacement configuration by a pivoting movement;
the configuration of all wheels in the disengaged/displacement configuration when both primary handgrips are lowered in the displacement configuration; and
the configuration of at least one wheel, preferably at least two wheels, in the wheel braking configuration when a pressure applied on at least one of the primary handgrips, already in the lowered configuration, exceeds a handgrip pressure threshold (wherein excessive pressure may be associated to a potential rollator user fall).

In an embodiment, the primary handgrips are operatively connected to a synchronisation device (i.e. the hazard detection system or the wheel braking assembly controller) and to the wheel braking assemblies, through the wheel braking assembly controller. In an embodiment, the connection is such that:
the configuration of at least one of the wheels, preferably all wheels, in the disengaged/displacement configuration, through the wheel braking assembly controller, if the two primary handgrips are simultaneously configured in the handgrip displacement configuration, i.e. they are lowered by a pivoting movement about an horizontal pivot axis, and;
the configuration of at least one wheel, preferably at least two wheels, in the wheel braking configuration when the pressure applied on at least one of the primary handgrips, already in the lowered configuration, exceeds a handgrip pressure threshold.

In an embodiment, each one of the handgrips includes a substantial horizontal component and a substantial vertical component, the vertical component being secured to the horizontal component and extending sensibly perpendicular to the horizontal component.

In an embodiment, the wheel braking system, which configures the wheel braking assemblies in the engaged and disengaged/displacement configurations respectively preventing and allowing rotation of the wheels, includes at least two components: a gear braking disk being mounted to a respective one of the wheels and a braking lever (or wheel stopper), mounted to a braking box and positioned relatively in face of the gear disk in a way that the two components can interact together in a cooperative way. The gear braking disk and the braking lever comprise a plurality of teeth and throats, respectively located on an outside perimeter of the gear braking disk and at a lower portion of the braking lever. The geometry of the tooth and throats can be selected in a manner such that they perfectly fit together with minimal force or pressure. In an embodiment, the complementary geometry of the tooth throat is susceptible to create a retaining effect when interacting with the wheel stopper which prevents accidental disengagement of the two components without external force being applied. In an embodiment, complimentary geometry of each component includes locking elements, for example “zipper type”, positioned on concentric circle arcs.

In an embodiment, the wheel braking system comprises:
a blocking gear (or gear disk) integrated to or mounted to at least one of the wheels, where the blocking gear having a disk shape attached to one of the wheel lateral face, which may cover the external part of the wheel and has on its circumference a number of protuberances; and
the wheel stopper includes teeth having a geometry compatible with one section of the circumference of the braking gear and a translating device that allows the teeth to interlock in the space left between the protuberances of the braking gear.

In an embodiment, the wheel stopper is made out of two jaws, one may be activated with the simultaneous pivoting movement of both primary handgrips in the handgrip displacement configuration while the second one may be activated when an excessive pressure is applied on at least one of the primary handgrips.

In an embodiment, the wheel braking system is configured to prevent the movement of the rollator at least in the longitudinal direction.

In an embodiment, configuration of both primary handgrips by a pivoting movement and under normal and limited pressure allows to configure the wheel braking system into the disengaged/displacement configuration allowing rotation of the wheels. In a case of a voluntarily or accidentally disengagement of at least one of the primary handgrip (in a way that the primary handgrip returns in its raised configuration, i.e. the handgrip braking configuration), the wheel braking system will automatically and immediately activate the braking of at least one but preferably two wheels, preventing the displacement of the rollator.

In an embodiment, the foldable rest seat can pivot and be folded over at least one of the side or pivot about one transversal bar located in between the two lateral side portions of the chassis of the multifunctional rollator. In an embodiment, the rest seat pivot axis is about horizontally positioned toward front plate in between the about two lateral side portions of the chassis with a pivot attached to the chassis.

In an embodiment, the foldable rest seat can be configured:
in a first substantially horizontal position, referred to as the seated position, to sit down if needed; and
in a second substantially vertical position, referred to as walking position, to position himself inside the U-shaped chassis structure, which area is cleared when the rest seat is in its about vertical position allowing the user to perform his daily task with supports surrounding him (i.e. the primary handgrips and the front handrail).

In an embodiment, the secondary handgrips include two symmetric horizontal supports, preferably adjustable in height, configured and positioned to allow the user to take hold thereon while sitting down or standing up and while allowing the user to stay within the U-shaped of the multifunctional rollator. The secondary handgrips can extend about horizontally toward front of the multifunctional rollator.

In an embodiment, the rollator includes a front handrail extending about vertically and being mounted to the front portion of the multifunctional rollator. The front handrail can be used as a backrest for the user when in the seated position. In an embodiment, the handrail inner surface (i.e. located inside the U-shaped chassis) is covered with a cushion for greater comfort.

In an embodiment, the wheel braking system is operatively connected to the fixed wheels of the rollator. In an embodiment, the configuration of the wheel braking system into the braking configuration is voluntarily accomplished by the user, under a controlled pressure, and/or is configured automatically by the wheel braking system controller in case a fall of the user is detected.

In an embodiment, a height of the rollator chassis can be adjusted and, more particularly, a height of the rest seat.

In an embodiment, an angle between the backrest, i.e. the front handrail, and the seat, configured in the seated configuration, can be adjusted to fit the user need or morphology. In an embodiment, the rollator is configured in a manner such that the backrest can be replaced quickly without tools with another particular backrest showing different features, for example replacing the backrest with a shaped molded backrest. The backrest can be of a hollow type or partially filled. In an upper section, it can have a handle/handgrip/lever/releasing device that allows to adjust the backrest configuration, for example to adjust its angle and/or its height. In an embodiment, control cable can extend through the backrest.

In an embodiment, the open structure, i.e. chassis viewed from a top plan view, is of the bottomless chair type and has a U-shaped defining a square or rectangular area with one side of the square or rectangular being removed (or unclosed). In an embodiment, the seat can be detachable and removable from the chassis.

The rollator can be operated into a transport chair mode or with the wheelchair configuration and in which the primary handgrips are swung forward allowing a caregiver to push the rollator while the user is seated on the seat. As mentioned above, the primary handgrips can be pivoted forwardly to clear lateral space surrounding the user while sitting down on the rest seat or using the multifunctional rollator as a transport or wheelchair. When configured in the frontward configuration, the primary handgrips can be used by the caregiver for the operation of the multifunctional rollator. Thus, the primary handgrips can be configured either in their normal rearward position which allows the user to operate the multifunctional rollator; or in their frontward position which frees the inside area of the multifunctional rollator while also acting as handlebar for the caregiver to operate the multifunctional rollator.

In an embodiment, the rollator includes at least one accessory receiving tube (i.e. a slide-in tube) mounted to the chassis. The accessory receiving tube can be mounted to the lateral side portions of the chassis, close to the rear end. The accessory receiving tube can be used to mount additional and complementary accessories. In an embodiment, the accessory receiving tube is positioned sensibly vertically at the rear end of each lateral side portion. The accessory receiving tube can have a circular, square or rectangular cross-section.

In an embodiment, the seat is operatively connected to an anti-roll back system. More particularly, the anti-roll back system is linked to a pivot of the rest seat and controls the wheels braking system in a manner such that the wheel braking system remains in the brake configuration, in the seated configuration of the seat, unless the user is seated in a safe position.

In an embodiment, each one of the wheels is rotatably mounted to a lower end of the chassis at a rotation axis.

In an embodiment, the control cables are mounted to a front plate of the front portion of the rollator chassis. In an embodiment, the control cables are multi-strand cables that allow physical transmission of the control commands.

In an embodiment, a plate is received on a front face of the front plate and which acts as an attachment face for the hazard control system (i.e. the wheel braking system controller) and/or the anti-roll back system.

In an embodiment, the chassis includes two lateral extensions, extending substantially parallel to one another but spaced apart and normal to the front portion of the chassis. The lateral extensions are configured to receive at least two pivots allowing for the tilting of the rest seat.

In an embodiment, the chassis includes two lateral surfaces, extending substantially parallel to one another but spaced apart and normal to the front portion of the chassis. The lateral surfaces are configured to receive a locking system for at least one of the pivotable handgrip frame supporting the primary handgrip. The lateral surfaces can also support pivots allowing the pivoting movement of the handgrip frame between the frontward configuration and the operative (rearward) configuration.

In an embodiment, the multifunctional rollator includes a fall detection system. The fall detection system can include at least:
a primary handgrip assembly which function is to collect information about the hand movement and the pressure/force applied by the user on the primary handgrips (i.e. the user hand behavior);
a wheel braking system operatively connected to at least two wheels;
a wheel braking system controller (i.e a hazard detection system) that mechanically receives and interprets the information from the primary handgrips (i.e. the primary handgrips position and the pressure applied thereon); and
mechanical or electrical connectors linking together all components to assure the transmission of the primary handgrip configuration (including the pressure applied thereon) to the wheel braking system controller.

In an embodiment, each one of the primary handgrip assemblies comprises:
a primary handgrip motion reading box;
a weight/pressure transmission rod;
a pivot mounting point on which the primary handgrip is attached;
a return device that biases the handgrip to its normal and rest position;
a handgrip pivot axis around the pivot of the weight/pressure transmission rod which may include a stopper that limits the pivoting movement of handgrip lever to a relatively horizontal position (the stopper being adjustable so that activated angle position of the handgrip be adjustable to user's morphology and comfort); and
sheaths, such as Teflon® lined sheaths, in which multi-strand control cables travel, the multi-strand cables being attached to the wheel braking system controller at another end thereof.

The primary handgrips can include at least one of the following characteristic:
a primary handgrip motion reading box which detects the user's hand behavior and transfers the information to the hazard detection system (HDS) (or wheel braking system controller);
a weight/pressure transmission rod positioned inside of the motion reading box having a return pressure that opposed to the pressure applied by the user on the primary handgrips;
a pivot point on which the primary handgrip can pivot (either upwardly or downwardly) which is slightly away from a physical transmission link (i.e. a multi-strand control cable) attachment point, which function is to transmit the handgrip displacement to the wheel braking system controller;
a return device (which can be a spring) connected to the weight/pressure transmission rod, which can include an adjustment device to adjust of the return force; and
a handgrip or lever pivoting on the pivot of the weight/pressure transmission rod and for which the activated angle, i.e. the angle defined by the handgrip in the lowered or handgrip displacement configuration, can be modified.

In an embodiment, the primary handgrips have two operating position (or configurations): a first position which corresponds to a handgrip braking configuration and a second position which corresponds to a handgrip displacement configuration.

In an embodiment, the wheel braking system controller (i.e. hazard detection system) mechanically/electrically interprets, using at least one relay, the information relative to an abnormal weight being applied on the primary handgrips. It can include a balancer plate pivoting on a transmission plate, which disengages the wheel braking system when and only when a normal pressure or weight is being applied simultaneously on the two handgrips, which are configured in the lowered configuration, i.e. the handgrip displacement configuration.

In an embodiment, the weight/pressure transmission rod is attached in a rotative way to the primary handgrip, which can show an angle in relation with a vertical axis. In an embodiment, the primary handgrip is towards a rear end of the chassis and can define and angle ranging from 0 to 60 degrees and, in an embodiment, from 25 to 35 degrees. In an embodiment, the weight/pressure transmission rod defines an angle of about 30 degrees with respect to the vertical axis and, in an embodiment, between 0 and 45 degrees.

It is appreciated that wheel braking assemblies can be of any type. In an embodiment, the wheel braking assemblies are activable via a physical transmission links such as multi-strand cables receiving instructions from to the wheel braking system controller (i.e. the hazard detection system).

A In an embodiment, the wheel braking system controller (i.e. the hazard detection system) integrate at least one of the following characteristics/features:
it mechanically interprets (using relay), the information relative to an abnormal weight/pressure transfer to the primary handgrips; and
includes a balancer pivoting on a transmission plate configured to disengaged wheel braking system when and only when a sufficient force or pressure is applied on both handgrips.

In an embodiment, the weight/pressure transmission rod on which the primary handgrips are attached via a pivot can adopt an angle relative to a vertical axis. This angle can be slightly inclined toward the rear end of the rollator, opposed to the front portion. This angle can be between 0 to 45 degrees or between 0 and −45 degrees and, in an alternative embodiment, between 0 to 30 degrees or between 0 to −30 degrees.

In an embodiment, the gear disks of the wheel braking assemblies can include:
a disk showing a plurality of external or internal indented profiles located on one side or on the inner or outer peripheral side. The gear braking disk can be mounted to flat wheel type with the indented profile being positioned on the outer peripheral side for a smaller wheel and on the inner peripheral side for a larger wheel;
a jaw or lever (i.e. a wheel stopper) having tooth with a complementary configuration that are engageable with the corresponding indented disk; and
an action device configured to selectively engage or disengage the two profiles.

In an embodiment, the anti-roll back system of the rollator is operatively connected to the seat. The seat is mounted to one or many foldable horizontal components and, when configured in a raised configuration, clears the inside volume of the U-shaped chassis. The anti-roll back system can include two (2) brake lifters, which can be hook-shaped, positioned symmetrically, extending across a central front plate of the chassis to interact with a balancer of the hazard detection system located advantageously on the opposite side of the front plate. In an embodiment, the lifters are fastened to the rest seat and pivots therewith. When a pressure greater than a set pressure threshold is applied on the seat, the lifters are lifting up both extremities of the central balancer which, via a transmission plate attached to it, pulls the braking physical transmission link (in this case the multi-strand control cables), which in turn disengage the wheel braking system of the multifunctional rollator. In an embodiment, the anti-roll back system can be used with wheels of any suitable diameters including wheelchair wheels.

In accordance with an aspect, there is provided a manufacturing method of an ambulatory system as described above and including an integrable rehabilitation device, by assembling, using well known assemblage methods and means, the constitutive components of the system and of the integrable device. The well-known methods and means can be selected from the group including: welding, collage, screwing, bolting, riveting, clipping and combination thereof.

In accordance with an aspect, there is provided a use of the system as described above for the escort and/or the safe and ergonomic rehabilitation of a person suffering from physical or intellectual impairment. During use, the handgrip frame including the primary handgrips can be positioned rearwardly, i.e. in the walking position, where the user safely controls the engagement/disengagement of the wheel braking system by simply depressing or pivoting of handgrips; or can be positioned forwardly when the user is in a seated position. In the frontward configuration of the primary handgrips, the caregiver can drive the rollator from front using the primary handgrips as guiding grip or device (equivalent to a back cane on a wheelchair) which allows the caregiver to easily push the user sitting on the rest seat configured in seated or horizontal position.

There is also provided a method for interactive mobility and/or rehabilitation assistance and/or social reinsertion and/or education and/or social reinsertion of a person showing limited physical and/or intellectual capacity. The method can include the following steps:
of travelling from point A to point B;
of travelling in a seated position, in an autonomous way (with the assistance of push rims for example) from point A to point B;
of travelling in a seated position, in an autonomous way (with the assistance of a caregiver using the primary handgrips in the frontward position) from point A to point B;
of realizing activities in the standing up position inside the U-shaped chassis;
to stand up from a seated position inside the area of operation to a standing up position using the secondary handgrips as a support for body weight; and
to sit down from a standing up position to a seated position within the surface of operation.

By using the method as described above, it may:
reduce the risks of a fall of the mass and/or the person while moving himself; and/or
reduce the injury risk of the rollator user; and/or
to help an handicapped person have higher autonomy or being completely autonomous in his displacement; and/or
to safely help a user in his rehabilitation/re-education process in an ergonomic way; and/or
to help a user using a step-by-step displacement behavior in its rehabilitation process by automatically alternating engagement/disengagement of the wheel braking system with the objective to assist weight transfer and feet sliding giving the user the required firm and stable support he needs to transfer an important part of his weight between each step; and/or
to give the user an easy access to non-adapted facilities or to environments not specifically adapted to the user's condition.

In an embodiment, the fall detection system of the rollator includes:
an assemblage of two primary handgrips (right and left);
a central system hazard detection system (or wheel braking system controller) that is detecting potential dangers; and
a wheel braking system, which can include disk brakes such as gear type;
these three (3) components are interconnected through physical transmission links, such as control cables, which transmit a movement initiated by the displacement of one of the primary handgrips.

In an embodiment, there is provided an integrable walk rehabilitation aid (walking re-education system/assembly) which includes:
at least two (2) symmetrical structures (or lateral frames) allowing the attachment of the walking re-education system to the lateral side portions of the rollator; and
an articulation device of the two (2) symmetrical structure including a re-educational device which can be positioned at hip height of the user;
wherein each of the components is configured to be attached to the multifunctional rollator and bearing the re-educational device permitting the linear displacement of the re-educational device parallel to the ground direction.

The walking re-education system/assembly can include at least one of the following characteristics/features:
two structures/frames positioned laterally with respect of the backrest of the rollator, each of the structures having at least two vertically positioned and relatively parallel members and at least 2 horizontally positioned members with at least one is a linear rail that allow a chariot to travel within, the chariot being linked to the re-education device;
the vertically positioned front member being configured at its lower end to be inserted into an accessory receiving tube, which can be located to the corresponding foot of the rollator and for which the second vertical (located at the rear end) is linked to a third set of wheels attached at a rotation axis; and
a re-educational equipment configured to generate an horizontal degree of liberty and rotation liberty which objective is to retain the user in case of a fall.

In an embodiment, the rear assembly of the multifunctional rollator is configured to received big wheels, i.e. wheelchair wheels, where wheels having circular rim coaxially attached to the wheel (i.e. push rims) which advantageously allows the user to propel himself in a well-known wheelchair.

In an embodiment, there is provided at least one primary handgrip assembly including a handgrip control box which is mounted directly or indirectly to the chassis through connection means. In an embodiment, a position or a configuration of the handgrip control box can be adjusted. The at least one primary handgrip assembly also includes a L shaped bended tube connected to the chassis and having a vertical upper extension with a threaded female insert adjustable in height on which the handgrip control box can be removably attached.

The handgrip control box can include a vertical lower extension having a threaded female insert adjustable in height which allows a removable attachment to a L-shaped bended tube connected to the chassis. In an embodiment, the cross dimensions of the lower extension correspond to those of a vertical sliding tube of the control box and allows sliding over, with low friction. The handgrip control box can include a lower bended extension removably and adjustably connected to a straight or bended hollow tube connected to the chassis of the rollator.

The rollator described below has a U-shaped chassis that may contribute to reducing congestion with the use of a rollator in everyday life. The rollator may include a fall detection system that may help reducing falls. The system is designed to automatically engage the braking system with the wheels in a situation of danger for the user. The rollator is also adaptable to each user in everyday life environments that are generally non-adapted to users that need mobility support.

In an embodiment, there is provided a wheel braking assembly controller, also referred to as the hazard detection system, for a mobility device, mounted on wheels such as a multifunctional rollator. The wheel braking assembly controller can be connected in a cooperative mode with any one of the following components of the multifunctional rollator:
at least one and, in an embodiment, two of the primary handgrip(s);
at least one and, in an embodiment, two of wheels braking assemblies; and
at least one foldable seat.

The wheel braking assembly controller can be configured in a way that:
in a walking mode, wherein the seat is a non-specific position:
   the mechanism interacts with the primary handgrip(s) and the wheel braking assembly(ies), and
   the mechanism initiates the engagement/disengagement of the wheels braking assembly(ies) only if the user/operator exercises pressure simultaneously on the two primary handgrips,
in seated mode, wherein the seat is about horizontal and the user/operator is seated or prepared to be seated:
   the mechanism interacts with the wheel braking assembly(ies) and the foldable seat, and
   the mechanism initiates the engagement/disengagement of the wheels braking assembly(ies) only if a preset minimum weight, i.e. a seat pressure greater equal to or greater than a seat pressure threshold, is applied and maintained on the seat. In a non-limitative embodiment, the preset minimum weight ranges between 15 to 100 pounds,
in fall recognition mode wherein the user\operator applies an abnormal pressure on at least one of the primary handgrip(s), i.e. a pressure equal to or exceeding a handrip pressure threshold,
   the mechanism interacts with the primary handgrip(s) and the wheel braking assembly(ies), and
   the mechanism initiates the engagement of the wheels braking assembly(ies) on at least two wheels.

In the walking mode, the wheel braking assembly controller can:
Configure at least one of the wheel braking assemblies and, in an embodiment, both wheel braking assemblies in the disengaged/displacement configuration wherein the wheels can freely rotate only if the two primary handgrips are simultaneously configured in the lowered configuration, i.e. the handgrip displacement configuration, preferably by a pivoting movement of the handgrips about an horizontal axis, and;
Configure at least one of the wheel braking assemblies and, in an embodiment, both wheel braking assemblies in the engaged/braking configuration as soon as the pressure equals or exceeds a handgrip pressure threshold on at least one of the primary handgrips.

According to a general aspect, there is provided a multifunctional rollator having an open or non-open structure allowing at least one of the following additional functionalities:
usable as a rollator;
usable as a walker;
usable as a transport chair;
usable as a wheelchair;
usable as an office wheeled chair;
having fall detection capability;
having re-educational or re-habilitation capability to be exploited by a professional or non-professional user;
offering an automatic braking system, that can be de-activated only when both hands are in contact with handgrips which represent an educative functionality helping the health professional to teach the right rollator usage;
offering a braking system that assures the user that in any time the rollator has its brakes on unless external and volunteered forces are applied correctly on handgrips and/or the user is safely seated;
offering a braking system that uses a large gear braking disk system that permits advantageous fine and efficient braking that minimizes the delay before brake activation;
offering an improved and advantageous tooth and throat geometry that reduces the floating effect of the gear braking mechanism in comparison with other gear braking systems which in turn reduces the delay before brake components engage with each other;
showing increased stability, especially when body weight is being transferred to the chassis which in turn reduces the potential overthrow of the multifunctional rollator;
integrating secondary handgrips that act as lateral support and that are adapted and finely adjustable to be use from the seated position or to the seated position;
integrating braking functionality to the rest seat where the brakes are applied unless the user is seated and safe in which condition the braking system is released this functionality acting as an integrated anti-roll back system;
integrating multi-adjustability that allows the multifunctional rollator to be finely adapted to the morphology of the user independently from right to left;
integrating multi-adjustability where the health professional can adjust the pressure or weight required to disengage the braking system which in other word is adjusting the fall detection system to fit user behavior, weight or morphology;
that shows reduced ground footprint and improved field of view in front of the multifunctional rollator which allows a reduced space congestion and a better visual detection of obstacles in front of the multifunctional rollator;
is foldable in a compact easy to store geometry;
that offer a frontal support while the user is standing up working on a counter height surface of work;
that adapt to a non-adapted environment particularly when used in a non-adapted toilet stall;
that shows a foldable rest seat that improved field of view when using the multifunctional rollator.
that shows primary handgrips capable of being toggled away for greater comfort and additional functionality when the multifunctional rollator is being used in its transport chair mode, its office wheeled chair mode or in its wheelchair configuration;
that introduce an advantageous anti-roll back system link to the rest seat of the multifunctional rollator and integrated into the chassis which does not show the habitual discomfort link to a detection system that would be installed underneath the seat;
that is lightweight and compact when folded;
where operating pressure of detection system are fully adjustable to adapt the behavior of any given user;
that introduce fine adjustability of all handgrips height with scaled indicator giving precise reading and data to the health professional;
that introduce a hazard detection system that interpret the user's hands behavior and determine whether or not the user is in a safe set of conditions (logical interpretation of hands behavior);
that gives the user a better lateral and frontal stability giving the fact that the user is travelling inside the chassis of the multifunctional rollator;
that shows a relatively centered centre of gravity especially link with the relative position of the user inside the chassis which result in reducing the potential overthrow of the multifunctional rollator when for example excessive pressure is applied which characterise the behavior of a user falling;
that shows an advantageous position of the user inside the chassis that make the user and multifunctional rollator combination a more compact combination reducing space congestion in public area;
that allows an optimal arm to body angle (via adjustable features) which allows the user a higher and better weight transfer to the multifunctional rollator which prevent overthrow and help in the re-education process;
that gives the care-giver sufficient space to push the multifunctional rollator when used in the transport chair mode or with wheelchair configuration, comparable to the space available with the use of a conventional wheelchair;
that gives the user sufficient space for foot when the multifunctional rollator is used in the transport chair mode;
that offer alternative propulsion mode when the multifunctional rollator is used into its wheelchair configuration (push rims);
that offer alternative propulsion mode when the multifunctional rollator is used with alternative retractable lever (paddling type of propulsion);
that offer in an alternative configuration a backrest adjustable in height and in angle or adjustable declination (relative to the rest seat);
that offers the capability to integrate many useful accessories via the use of the accessories slide-in tube without any technical assistance;
that offers accessories to improve comfort of the user comprising for example a wheelchair type foldable feet rest support that is foldable in case the user propels himself with his feet when rollator is used in the transport chair mode or in the wheelchair mode;
that offers accessories relative to physical or medical condition of the user such as an oxygen tank support, a solute pole, a medical monitor to name a few;
that offers accessories relative to rehabilitation or walking re-education such as the physiotherapy rear extension with twist belt that support weight of the user while being re-educate. Given the fact that in rehabilitation the number of training hours reduces the time for rehabilitation, given the fact that falls occurs mainly at the beginning of the rehabilitation process, given the fact that more and more person will required rehabilitation, given the fact that physiotherapist needs will increase in the near future and that the physiotherapist availability will be limited, this equipment will be appreciated;
that offers an advantageous alternative to the existing rehabilitation solutions that are most of the time heavy, not well adapted and cumbersome;
that, given the safety functionality of the physiotherapy rear extension, can be used with minimal supervision from a physiotherapist;
that given the weight support integrated functionality allows the physiotherapy rear extension together with the twist belt and the weight support device to be adjusted to support between 20 to 100% of the user's body weight which help in progressively reloading the user's legs;
that offers accessories relative to commodity usage such as a cup holder, a telephone holder, an electronic tablet holder or lateral storage bags that can be obtained separately and quickly installed without tools or technical assistance;
that allows the use of public adapted or non-adapted public or private toilet stalls given the ground to front plate clearance that allows the multifunctional rollator to be rolled over a regular toilet bowl and allows the user to use the secondary handgrips or support to take hold while sitting down the toilet seat so that the multifunctional rollator is no longer disturbing the area surrounding the toilet and becomes a tool that allows the user to use non-adapted toilet stall;
that permit the right height adjustment of the secondary handgrips in order that they are at the perfect height according to user morphology in order to have the perfect height to sit on the toilet seat.

According to a general aspect, there is provided a multifunctional rollator allowing a user to move himself in an autonomous way, or semi-autonomous way and to use the non-adapted daily life commodities keeping acceptable ergonomic positioning while the user position himself in a cleared space inside the rollator “U” shaped chassis of the multifunctional rollator.

According to a general aspect, there is provided a multifunctional rollator allowing the user to move himself in an autonomous and safe way in a seated or walking position while having access to non-adapted to handicapped person daily life commodity, the multifunctional rollator being equipped with 4 vertical members extending toward the ground and having at least one wheel on each extension and equipped with at least one of the following device:
a blocking/braking mechanism allowing to release the blockage/braking of at least one of the wheel under controlled pressure or weight and/or the blockage/braking of at least one of the wheels in case a fall occurs;
a foldable rest seat attached to the chassis having two preferred position, a first position said the about horizontal position where the user can sit down in case of need and a second position, said the about vertical position where the interior of the “U” shaped chassis is cleared and where the user can position himself to walk around assisted by the multifunctional rollator realizing his daily tasks; and
a second set of supports, the secondary handgrips, on which the user can take hold while standing up or sitting down.

According to a general aspect, there is provided a multifunctional rollator, allowing the user to move himself in an autonomous and safe way in a seated or walking position while having access to non-adapted to handicapped person daily life commodities, the multifunctional rollator being equipped with at least one of the following devices:
a blocking/braking system allowing to release the blockage/braking of at least one of the wheel under controlled pressure or weight and/or the blockage/braking of at least one of the wheels in case a fall occurs;
a foldable rest seat attached to the chassis having two preferred position, a first position said the about horizontal position where the user can sit down in case of need and a second position, said the about vertical position where the interior of the “U” shaped chassis is cleared and where the user can position himself to walk around assisted by the multifunctional rollator realizing his daily tasks;
a second set of support, the secondary handgrips, on which the user can take hold while standing up or sitting down; and
a pair of propulsion wheels with push rims located laterally on each rear side of the multifunctional rollator, the propulsion wheels being destined to ease the displacement of the multifunctional rollator by the use of the push rims coaxially attached to the wheels, the push rims can be those defined in the International patent application WO2013CA50521, which is incorporated herein by reference.

According to a general aspect, there is provided a multifunctional rollator as described above, which further includes:
a frontal assembly and two lateral rear side assemblies positioned sensibly at about right angle with the frontal assembly which defined a “U” shaped cleared and empty area;
a mobility system (supporting elements or chassis of the multifunctional rollator in contact with the ground offering reduced friction and increased support and stability) including preferably 4 wheels in contact with the ground in which:
2 wheels positioned in the frontal part, each of the 2 wheels being attached at the bottom of each lateral assembly and at proximity of the front part or attached to each side of the frontal part, and
2 wheels positioned on the rear part where each wheel is attached to the bottom part of each of the lateral assembly;
at least 2 support handgrips (secondary handgrips), preferably adjustable in height, attached about symmetrically to the rear lateral assembly of the system;
a foldable rest seat, folding against backrest or folding left to right or right to left against one of the lateral assembly, in the preferred embodiment, the rest seat is advantageously foldable against the backrest located on the frontal part; and
a blocking/de-blocking device comprising 2 handgrips (primary handgrips) that are preferably mounted on two swing arms and are configured to control the blocking/de-blocking of the system wheels. Each of the handgrips being linked to a braking system which controls at least one of the system's wheel, the braking system being disengaged only if both primary handgrips are in the disengaged position and the braking system being engaged if only one of the handgrip is in the disengaged position.

In an embodiment, the fall detection/braking system is configured to realize at least one of the following functions:
the braking/blocking of at least one but preferably of two wheels when only one of the primary handgrip is activated preferably by rotation;
the releasing (or de-blocking) of all the rollator wheels when both primary handgrip are activated;
the braking/blocking of at least one but preferably two wheels when the pressure applied on the already activated primary handgrip exceeds a pre-determined pressure on at least one of the primary handgrip (excessive pressure=falls); and
the braking/blocking of at least one but preferably two wheels when the pressure applied on both (2) already activated primary handgrip exceed a pre-determined pressure (excessive pressure=falls).

In an embodiment, the detection/braking system includes two primary handgrips interactively link to a synchronisation device (the hazard detection system) that links to at least one of the braking/blocking devices (the gear braking system) of at least one of the system's wheels. The detection/braking system can be configured in a way that:
the de-blocking/de-braking action of at least one but preferably all of the wheels can be done through the synchronization device (hazard detection system) only if two primary handgrips are simultaneously activated by preferably rotation around their respective about horizontal axis, and;
the blocking/braking of at least one of the wheels but preferably two wheels as soon as the pressure exceed a maximum pre-set value on at least one of the primary handgrips.

Each of the primary handgrip can comprise an about horizontal and an about vertical component, the vertical component being advantageously secured to the horizontal component and being sensibly perpendicular to the horizontal component of the handgrip.

The wheel braking system, that blocks or de-blocks the wheels from turning, is attached to at least one of the wheels and includes at least 2 blocking elements: the first element, the gear braking disk being attached to one of the wheels and the second element, the braking lever, being attached to the back of the braking box and positioned relatively in face of the first element in a way that the two can interact together in a cooperative way.

The gear braking disk and the braking lever can comprise a plurality of teeth and throats located on the outside perimeter of the gear braking disk and on the bottom portion of the braking lever, the geometry of the tooth and throats are so that they perfectly fit together with minimal force or pressure. The complementary geometry of the tooth throat is susceptible to create a retaining effect when interacting together which prevent accidental disengagement of the two components without external force being applied. The complimentary geometry of each element is locking elements, for example “zipper type”, positioned on concentric circle arcs.

The wheel braking system can comprise:
a blocking gear integrated to at least one of the wheels, where the gear having a disk shape attached to one of the wheel lateral face, preferably covering the external part of the wheel and having on its circumference a number of protuberances; and
the corresponding teeth having a geometry compatible with one section of the circumference of the braking gear and a translating device that allows the teeth to interlock in the space left between the protuberance of the braking gear.

The first element can be made out of two jaws, one may be activated with the simultaneous rotation of both primary handgrips while the second one may be activated when excessive pressure is applied on at least one of the primary handgrip. The wheel braking system of at least one of the system's wheel will brake the movement of the system in at least one of the longitudinal direction.

In an embodiment, the activation of the 2 primary handgrips by rotation, and under normal and limited pressure, allows to free the wheel's rotation from being blocked by the wheel braking system and, in a case of a voluntarily or accidentally de-engagement of at least one of the primary handgrip (in a way that the primary handgrip returns in its non-activated position) the automatic braking system will immediately activate the braking of at least one but preferably two wheels, stopping the rollator from moving.

In an embodiment, the foldable rest seat can rotate and be folded over at least one of the side or rotate around one transversal bar located in between the two lateral portion of the front plate assembly of the chassis of the multifunctional rollator and where, in the preferred embodiment, the rest seat rotation axis is about horizontally positioned towards the front plate in between the about two lateral portions of the chassis with a pivot attached to the lateral part of the front plate assembly of the multifunctional rollator.

In an embodiment, the foldable rest seat allows the user:
in a first about horizontal position, called the seated position, to sit down in case of need; and
in a second about vertical position, called the walking position, to position himself inside the U-shaped chassis structure of the multifunctional rollator, which area is cleared when the rest seat is in its about vertical position allowing the user to perform daily tasks with supports surrounding him (primary handgrips and front handrail).

In an embodiment, the secondary handgrips constituted of two symmetric horizontal support, preferably adjustable in height, configured and positioned to allow the user to take hold on the secondary handgrips while sitting down or standing up and while allowing the user to stay within the U-shaped of the multifunctional rollator.

In an embodiment, the substantially horizontal portion of the secondary handgrip extends about horizontally toward the front of the multifunctional rollator.

In an embodiment, a front handrail positioned about vertically is attached to preferably the front part of the multifunctional rollator, the front handrail also acting as a backrest for the user when in seated position and where the handrail inner surface (inside the U-shape chassis) is covered with a cushion for greater comfort.

In an embodiment, the at least one wheel equipped with the braking system is preferably a fixed wheel.

In an embodiment, the multifunctional rollator is configured in order that:
the blocking/braking of at least one wheel equipped with a wheel braking system is voluntarily accomplished by the user, under a controlled pressure; and/or
the multifunctional rollator automatically brakes the wheels of the multifunctional rollator in case of a fall from the user.

In an embodiment, the multifunctional rollator is configured to allow the height adjustment of the rest seat.

In an embodiment, the multifunctional rollator is configured so that the backrest (front handrail) angle relative to the seat rest in the about horizontal position can be adjusted to fit the user need or morphology and is also configured so that the backrest can be replaced quickly without tools with another particular back rest showing different features, for example replacing the back rest with a shaped molded backrest.

In an embodiment, the open structure is of the bottomless chair type.

In an embodiment, the open structure from bird view has a U-shaped covering a square or rectangular area with one side of the square or rectangular being removed.

In an embodiment, the rollator can be operated into a transport chair mode or with the wheelchair configuration and in which the primary handgrips are swung frontward allowing a care giver to push the rollator while the user is seated in.

In an embodiment, the multifunctional rollator is configured to receive rotation elements allowing the rest seat to be flipped toward the backrest.

In an embodiment, the multifunctional rollator is configured so that primary handgrips can be swung away to clear lateral space surrounding the user while sitting down on the rest seat or using the multifunctional rollator as a transport or wheelchair, advantageously the primary handgrips in the forward position are used advantageously for the operation by a care giver of the multifunctional rollator.

In an embodiment, the multifunctional rollator is configured to:
receive some rotation axis attachment points for tilting support members (of the swing arm type) which;
in their normal rearward position supports the primary handgrips assembly and allow the user to operate the multifunctional rollator; and
in the frontward position liberates the inside area of the multifunctional rollator while also acting as handlebar for the care-giver to operate the multifunctional rollator.

In an embodiment, the multifunctional rollator is further equipped with slide-in tube at the rear portion of the multifunctional rollator, where the slide-in tube allows for the attachment of additional and complementary accessories and where, in the preferred embodiment, the slide-in tube is positioned sensibly vertically on the rear end of each lateral assembly, the slide-in tube showing a circular, or advantageously square or rectangular shape.

In an embodiment, the rest seat located at the rollator front part, is configured preferably so that an anti-roll back system is linked to a pivot of the rest seat and controls the wheels braking system in a matter that the wheel's braking system stays engaged unless the user is seated in a safe position.

In an embodiment, each of the bottom lower extensions are configured to receive the rotation axis of the wheels.

In an embodiment, the multifunctional rollator includes a backrest of hollow type or partially filled that can have on its upper section a handle/lever/releasing device that allows to adjust the backrest configuration for example to adjust the angle and/or the height of the backrest.

In an embodiment, the front plate assembly is configured so that physical transmission links (multi-strand cable) can travel through.

In an embodiment, the front plate assembly has in its internal portion a face in contact with the back of the user and an opposite face (facing outside of chassis) that is configured to receive a plate that can receive at least one protuberance like a plate sensibly perpendicular to the opposite face and acting as an attachment face for the hazard control system and/or the anti-roll back system.

In an embodiment, the lateral assembly having at least 2 lateral surfaces about parallel to each other and sensibly perpendicular to the front assembly, are configured to receive at least two pivots allowing for the tilting of the rest seat.

In an embodiment, the lateral assembly having at least 2 lateral surfaces about parallel to each other and sensibly perpendicular to the front assembly, are configured to receive a locking system for at least one of the swing arms supporting the primary handgrips.

In an embodiment, the multifunctional rollator further comprises a fall detection system, the fall detection system comprising at least:
a primary handgrip assembly which function is to collect information on the hands movement and on the pressure/force applied by the user on the primary handgrips (user's hands behavior);
a wheel braking system of at least two wheels;
a central system (hazard detection system) that mechanically receive and interpret the information received from the primary handgrips (the primary handgrips position and the pressure applied); and
all components being linked together via some physical transmission links such as multi-strand cables that assure the right transmission of movement generated by the primary handgrips user's operation and for which sensitivity can be advantageously adjusted.

In an embodiment, the primary handgrip assembly comprises:
a primary handgrip motion reading box;
a weight/pressure transmission rod;
a pivot mounting point on which the primary handgrip is attached;
a return device that bring back the handgrip to its normal, rest position said the return device;
a handgrip pivoting around the pivot of the weight/pressure transmission rod which advantageously shows a stopper that limit the handgrip lever to a relatively horizontal position (the stopper being adjustable so that activated angle position of the handgrip be adjustable to user's morphology and comfort); and
a Teflon®-lined sheath in which a multi-strand cable travels, the multi-strand cable being attached to the hazard detection system on the other end.

In an embodiment, the primary handgrips include at least one of the following characteristics:
a primary handgrip motion reading box which read the user's hand behavior and transfer the information to the hazard detection system (HDS);
a weight/pressure transmission rod positioned inside of the motion reading box which shows a pre-determined return pressure that opposed to the pressure applied by the user on the primary handgrips;
a pivot point on which the primary handgrip can pivot (up or down) which is slightly away from a physical transmission link (multi-strand cable) attachment point which function is to transmit the handgrip displacement to the HDS;
a return device connected to the weight/pressure transmission rod which is advantageously a spring that can advantageously be equipped with an adjustment device permitting the adjustment of the return force; and
a handgrip or lever pivoting on the pivot of the weight/pressure transmission rod for which the activated angle can be modified.

In an embodiment, the primary handgrip has two operating positions, the first position assures that at least one of the wheels is locked/brake and a second position that assures that at least one but preferably all the wheels are free to rotate.

In an embodiment, the wheels braking/blocking system of at least two wheels can be of any type but said system being preferably actionable via a physical transmission link that is for example a multi-strand cable transmitting a movement to the central system (hazard detection system) which in turn transmits the moving authorization to the wheel breaking system.

In an embodiment, the central system (hazard detection system) integrates at least one of the following characteristics:
it mechanically interprets (using relay), the information relative to an abnormal weight/pressure transfer to the handgrips; and
includes a balancer pivoting on a transmission plate configured to disengaged wheel braking system when and only when a sufficient force or pressure is applied on both handgrips.

In an embodiment, the weight/pressure transmission rod on which the primary handgrip is attached via a pivot advantageously adopts an angle relative to vertical and is advantageously slightly inclined toward the back of the rollator in an opposite direction from the backrest.

In an embodiment, the weight/pressure transmission rod on which the primary handgrip is attached via a pivot can adopt an angle relative to the vertical in between 0 to 45 degrees or between 0 and −45 degrees.

In an embodiment, the weight/pressure transmission rod on which the primary handgrip is attached via a pivot is showing and angle to the vertical of location comprised in between 0 to 30 degrees or between 0 to −30 degrees.

According to a general aspect, there is further provided a disk gear braking system for emergency braking for a mobility aid, that may be for example a multifunctional rollator, the braking system including:
a disk showing a plurality of external or internal indented profile located on one side or on the inner or outer peripheral of the gear braking disk which is preferably connected to a flat wheel type, the indented profile being advantageously positioned on the outer peripheral for a smaller wheel and advantageously positioned on the inner peripheral for a larger wheel;
a jaw or lever showing tooth with complementary configuration that engage when needed with the corresponding indented disk; and
an action device configure so that the two profiles engaged in each other or disengaged when a user wish to free wheels movement.

According to a general aspect, there is further provided a disk/pinion braking system wherein the indented profile geometry is as defined on FIGS. 8 and 25.

According to a general aspect, there is further provided an anti-roll back system (advantageously linked to the rest seat), for a mobility-aid, noticeably for a multifunctional rollator having preferably a U-shape, showing at least 3 wheels and preferably 4 wheels, the anti-roll back system comprising:
a rest seat in which can be located one or many foldable horizontal element(s) that when fold cleared the inside volume of the U-shaped chassis; and
2 lifters preferably of a hook shape, advantageously positioned symmetrically, that extend across a central front plate of the multifunctional rollator to interact with the balancer of the hazard detection system located advantageously on the opposite side of the frontal plate the lifters being fastened to the rest seat which pivots are attached to the multifunctional rollator in a way that when the rest seat is forced to an about horizontal position, the lifters are lifting up both extremities of the central balancer which, via a transmission plate attached to it, pulls the braking physical transmission link (in this case the multi-strand cables) that disengaged the wheel braking system of the multifunctional rollator.

In an embodiment, the elements are configured in a way that the anti-roll back system is usable with a multifunctional rollator equipped with big wheels (wheelchair wheels) so that the blocking operation be done within the wheel axis and in line with a radius line.

According to a general aspect, there is further provided a primary handgrip device for a mobility aid such as for a multifunctional rollator having preferably a U-shape, the handgrip device having:
a motion handgrip reading box;
a weight/pressure transmission rod;
a pivot point on which the handgrip can tilt or pivot;
a return device that return handgrip to its normal non-depressed position, the return device (preferably a spring) being preferably equipped with an adjustment device that allow to adjust the return force;
a handgrip that pivot on the pivoting point of the weight/pressure transmission rod which integrate a stopper limiting the handgrip to a relative horizontal position when engaged (the stopper being fixed or being adjustable so that engaged angle can be adjust according to user comfort); and
a Lined sheath and physical transmission link (multi-strand cable) that links the system to the hazard control system.

In an embodiment, the central device (hazard detection system) mechanically interprets, using at least one relay, the information relative to an abnormal weight being applied on the handgrips and advantageously include a balancer pivoting on a transmission plate which disengaged the wheel braking system when and only when a normal pressure or weight is being applied simultaneously on the 2 handgrips.

The handgrip control box can be directly or indirectly, in a adjustable or in a non-adjustable way, connected to the chassis of the rollator by means of a connection means.

The handgrip control box can have a vertical lower extension of the handgrip control box with a threaded female insert adjustable in eight that permit the removable attachment to a L-shaped bended tube connected to the chassis, the cross dimensions of the lower extension corresponding to those of the vertical sliding tube of the control box and allowing sliding over, preferably with minimum friction.

The handgrip control box can have a L-shaped bended tube connected to the chassis and having a vertical upper extension with a threaded female insert adjustable in eight that permits the removable attachment of the handgrip control box, the cross dimensions of the L shaped bended tube corresponding to those of vertical sliding tube of the control box and allows slide over with minimum friction.

The handgrip control box can have a lower bended extension removable and adjustably connected to a straight or bended hollow tube connected to the chassis of the rollator.

In an embodiment, the weight/pressure transmission rod is attached in a rotative way to the action handgrip (primary handgrip) which can advantageously show an angle in relation with the vertical and is advantageously inclined toward an opposite direction from the backrest, the angle ranging preferably from 0 to 60 degrees.

In an embodiment, the weight/pressure transmission rod shows an angle in relation with the vertical and is advantageously inclined in a direction opposite to the backrest, the angle ranging preferably from 25 to 35 degrees.

In an embodiment, the weight/pressure transmission rod shows an angle preferably about 30 degrees from vertical.

In an embodiment, the weight/pressure transmission rod adopts an angle in relation with the vertical in between 0 and 45 degrees.

According to a general aspect, there is also provided a manufacturing method of an ambulatory system as defined above and of an integrable rehabilitation device, by assembling, using well known assemblage methods and means, the constitutive element of the system and of the integrable device.

The well-known method and means of assemblage can be selected in the assemblage group constituted by: welding, collage, screwing, bolting, riveting, clipping and combination of the later means.

According to a general aspect, there is also provided the use of a system as described above, wherein the use of a manufacturing system according to one of the method or mean also defined above for the escort and/or the safe and ergonomic rehabilitation of a person suffering from physical or intellectual impairment.

The use of the system can be carried out according to two positions:
with primary handgrip swing arms being tilted rearward say the walking position, the user safely controls the blocking and de-blocking of the wheel braking system by simply depressing or rotating of handgrips;
with primary handgrip swing arms being tilt forwardly say the seated position, a care-giver can drive the system from front using the primary handgrips as guiding grip or device (equivalent to a back cane on a wheelchair) which allow the care giver to easily push the user sitting on the rest seat in seated or horizontal position.

According to a general aspect, there is also provided a method for interactive mobility and/or rehabilitation assistance and/or social reinsertion and/or education and/or social reinsertion of a person showing limited physical and/or intellectual capacity. The method can include the following steps:
of travelling from point A to point B;
of travelling in a seated position, in an autonomous way (with the assistance of push rims for example) from point A to point B;
of travelling in a seated position, in an autonomous way (with the assistance of a care giver using the primary handgrip in the frontward position) from point A to point B;
of realizing activities in the standing up position inside the Safe U-shape chassis defined by the internal structure system;
to stand up from a seated position inside the area of operation to a standing up position using the secondary handgrip as a support for body weight; and
to sit down from a standing up position to a seated position within the surface of operation.

In an embodiment, the use of the method can be used to:
reduce the falls risk of the mass and/or the person while moving himself; and/or
reduce the injury risk of the user; and/or
help an handicapped person to have higher autonomy or being completely autonomous in his displacement; and/or
safely help a user in his rehabilitation/re-education process in an ergonomic way; and/or
help a user using a step-by-step displacement behavior in its rehabilitation process by automatically alternating blocking and de-blocking with the objective to assist weight transfer and feet sliding giving the user the required firm and stable support he needs to transfer an important part of his weight between each step; and/or
give the user an easy access to non-adapted facility or to environment not specifically adapted to the user condition.

According to another general aspect, there is also provided a chassis of a multifunctional rollator, wherein the structure is of bottomless chair type. The chassis can be configured to receive rotation element that allows to have a removable chair bottom or foldable chair bottom that can be folded or tilted against the backrest of the chair. The chassis can be configured in a manner such that it can receive some rotation axis for swing arms attachment. The chassis can be configured in a manner such that it can receive a sliding device sensibly vertical on each foot rod, the sliding device being preferably of circular and advantageously of square or rectangular section. The chassis can be configured at the lower part of the backrest to receive an anti-roll back system. In an embodiment, the lower portion of each foot of the chair is configured to receive a rotation axis for the wheels.

In an embodiment, the backrest is hollow or partially filled so that the upper portion of the backrest keeps the possibility to fit a handgrip or handle.

In an embodiment, the lower portion of the backrest is filled and configured to allow transmission cables to go through.

In an embodiment, the opposite face of the backrest is configured to receive at least one protuberance such as a plate sensibly perpendicular to the backrest which allows for the attachment of the anti-roll back system.

In an embodiment, the chassis has at least 2 lateral surfaces about parallel to each other and sensibly perpendicular to the backrest and configured so it can support pivots which objective is to permit the tilting of the swing arms that act as conduct handlebars for the care-giver.

In an embodiment, the chassis has at least two lateral surfaces about parallel to each other and sensibly positioned perpendicular to the backrest and configured so they can support the blocking or retaining device of the swing arms.

In an embodiment, the chassis has at least one complementary configuration that permit the attachment, preferably in a non-permanent way of advantageous complementary accessories.

According to another general aspect, there is also provided a fall detection system including:
an assemblage of two primary handgrips (right and left);
a central system hazard detection system that is controlling dangers; and
a wheel blocking system, preferably using disk brake and advantageously of the gear type;
the 3 elements being interconnected with physical transmission links which transmit a movement initiated by the displacement of one of the primary handgrip.

In an embodiment, the detection system has the configuration shown in FIGS. 9 and 10 and operates according to the logical diagram shown in FIG. 11 and for which the elements interconnection together with their functioning is shown in FIGS. 12, 13, and 14.

According to another general aspect, there is also provided a pinion braking/blocking system for emergency braking of a multifunctional rollator characterised by a reduced engaging delay and/or a reduced engaging pressure and/or a reduced bouncing effect that are typically associated with gear type braking system. The pinion braking/blocking system for emergency braking can have the configuration shown in FIG. 25.

According to another general aspect, there is also provided an integrable walk rehabilitation aid which includes:
at least 2 symmetrical structure allowing the attachment of the device to the lateral section or a rollator as defined by anyone of the claims 1 to 59 each of the structures being advantageously link to or constituting the integrality of the lateral sides of the rollator; and
an articulation device of the 2-symmetrical structure including a re-educational device which is positioned preferably at hip height of the user;
each of the parts being configured to be attached to the multifunctional rollator and bearing the re-educational device permitting the linear displacement of the re-educational device parallel to the ground directions.

The integrable walk rehabilitation aid can include at least one of the following characteristics:
two structures positioned laterally in respect of the backrest of the rollator each of the structures having at least two vertically positioned and relatively parallel members and advantageously at least 2 horizontally positioned members with at least one is a linear rail that permit a chariot to travel within, the chariot being link to the re-education device;
the vertically positioned front member being configured at its bottom to be inserted into a tube (the accessories slide-in tube) located preferably to the corresponding foot of the rollator and for which the second vertical (located at the rear end) is linked to a third set of wheels attached to a rotation axis; and
a re-educational equipment configured to generate an horizontal degree of liberty and rotation liberty which objective is to retain the user in case of a fall.

In an embodiment, the rear assembly of the multifunctional rollator is configured to received big wheels (wheelchair wheels) where wheels having circular rime coaxially attached to the wheel (push rims) which advantageously allows the user to propel himself in a well-known wheelchair common method well described in prior art.

According to another general aspect, there is also provided a hazard and synchronisation mechanism (also named the hazard detection system), for a mobility device, mounted on wheels such as a multifunctional rollator. The mechanism can be connected in a cooperative mode with following components of the multifunctional rollator:

A. at least one, preferably at least 2, active primary handgrip(s);

B. at least one, preferably 2, wheels braking devices; and

C. at least one foldable seat.

The hazard and synchronization device can be configured in a way that:
in walking mode wherein the seat is a non specific position:
   the mechanism interacts with components A and B, and
   the mechanism initiates de-blocking/de-braking action to the breaking device only if the user/operator exercises pressure simultaneously on the two handgrips,
in seated mode, wherein the seat is about horizontal and the user/operator seated or prepare to be seated on it:
   the mechanism interacts with components B and C, and
   the mechanism initiates de-blocking/de-braking action to the breaking device only if a preset minimum weight is applied and maintained on the seat, the preset minimum weight ranging, preferably from 15 to 100 pounds,
in fall recognition mode wherein the user\operator applies an abnormal pressure on at least one handgrip,
   the mechanism interacts with components A and B, and
   the mechanism initiates the blocking/braking action on at least two wheels.

In an embodiment, the hazard and synchronisation device, such as a mechanism, (also named the hazard detection system), can carry out, in walking mode:
the de-blocking/de-braking action of at least one, but preferably all of the wheels can be done through the synchronization device (hazard detection system) only if two primary handgrips are simultaneously activated, preferably by rotation around there respective about horizontal axis, and;
the blocking/braking of at least one of the wheels, but preferably of two wheels, is imitated by the synchronisation mechanism as soon as the pressure exceeds a maximum pre-set value on at least one of the primary handgrip.

### BRIEF DESCRIPTION OF THE DRAWINGS

FIGS. 1A, 1B and 1C are respectively a front perspective view, a left side elevation view and a front elevation view of a rollator in accordance with an illustrative embodiment, with a seat frame configured in a seated configuration.

FIG. 2 is a top plan view of the rollator shown in FIGS. 1A, 1B, and 1C, with the seat frame configured in the raised configuration and showing inside clearance within a seat/walker receiving area.

FIG. 3 is a rear perspective view of the rollator shown in FIGS. 1A, 1B, and 1C, wherein a handgrip frame is configured in a frontward configuration.

FIG. 4A is a bottom plan view of the rollator shown in FIGS. 1A, 1B, and 1C, with lateral side portions of a chassis in a partially compacted configuration and the seat frame in a raised configuration.

FIG. 4B is a rear perspective view of the rollator shown in FIGS. 1A, 1B, and 1C in the configuration of FIG. 4A.

FIG. 4C is an enlarged view of a pivotable connection between a lateral side portion and a front portion of the chassis including a chassis pivoting lock assembly.

FIGS. 5A and 5B are respectively a front perspective view and a left side elevation view of the rollator shown in FIGS. 1A, 1B, and 1C with the lateral side portions in the compacted configuration and a seat in the raised configuration.

FIG. 5C is a left side elevation view of the rollator of FIGS. 5A and 5B in the compacted configuration, tilted forwardly and ready for transport from one location to another.

FIG. 6A is a rear perspective view of the rollator shown in FIGS. 1A, 1B, and 1C with the seat in the raised configuration.

FIG. 6B is an enlarged view of a secondary handgrip assembly of the rollator of FIG. 6A.

FIG. 7A is a rear perspective view of a handgrip assembly of the rollator shown in FIGS. 1A, 1B, and 1C with an inner cover removed.

FIG. 7B is a right-side elevation view of the handgrip assembly of FIG. 7A.

FIG. 8A is a right-side elevation view of a fixed wheel and its wheel braking assembly of the rollator shown in FIGS. 1A, 1B, and 1C, wherein a cover portion is removed.

FIG. 8B is a left side elevation view of the fixed wheel and its wheel braking assembly of FIG. 8A.

FIG. 8C is an enlarged view of the wheel braking assembly of FIG. 8A configured in a wheel braking configuration.

FIGS. 9A and 9B are respectively a front perspective view and a front elevation view of the rollator shown in FIGS. 1A, 1B, and 1C, showing a first embodiment of a wheel braking assembly controller.

FIG. 9C is an enlarged perspective view of a right section of the wheel braking assembly controller of FIG. 9A.

FIG. 9D is an enlarged front elevation view of the wheel braking assembly controller of FIG. 9B.

FIG. 9E is a front perspective view of the rollator including a wheel braking assembly controller in accordance with a second embodiment.

FIG. 9F is a front elevation view the rollator including the wheel braking assembly controller shown in FIG. 9E.

FIG. 9G is an enlarged view of a section of the wheel braking assembly controller shown in FIG. 9E.

FIG. 9H is a front elevation view, enlarged, of the wheel braking assembly controller shown in FIG. 9F.

FIG. 9I is a front perspective view of the rollator including a wheel braking assembly controller in accordance with a third embodiment.

FIG. 9J is an enlarged view of a section of the wheel braking assembly controller shown in FIG. 9i.

FIG. 9K is a front elevation view, enlarged, of the wheel braking assembly controller shown in FIG. 9I.

FIG. 9L is a front elevation of the rollator including the wheel braking assembly controller shown in FIG. 9I.

FIG. 9M is a sectional view taken along cross-section lines A-A of FIG. 9L of the rollator.

FIG. 9N is an enlarged view of a section of the wheel braking assembly controller shown in FIG. 9M, showing a portion of an anti-roll back system.

FIG. 10A is a front elevation view of the rollator shown in FIGS. 1A, 1B, and 1C showing the anti-roll back system in the wheel braking assembly controller.

FIG. 10B is a sectional view taken along cross-section lines A-A of FIG. 10A of the rollator.

FIG. 10C is an enlarged view of FIG. 10B.

FIG. 11 is a flowchart of a safety condition recognition and identification in relation with a user's behavior.

FIG. 12 is a schematic representation of a wheel braking system including the wheel braking controller shown in FIGS. 9A to 9D, showing a safety condition when only one of a handgrip is configured in a displacement/walking configuration.

FIG. 13 is a schematic representation of the wheel braking system including the wheel braking controller shown in FIGS. 9A to 9D, showing a safety condition when the pair of handgrips is configured in the displacement/walking configuration and no excessive pressure is applied on the handgrips.

FIG. 14 is a schematic representation of the wheel braking system including the wheel braking controller shown in FIGS. 9A to 9D, showing a safety condition when an excessive pressure is applied on at least one of the handgrips.

FIG. 15A is a schematic left side elevation view of a user with a rollator of the prior art and FIG. 15B is a schematic left side elevation view of a user with the rollator of FIG. 1A, specifically illustrating an arm angle, blind spot, driving position and force vector generated by a user of the rollator.

FIG. 16A is a schematic top plan view of a user with a rollator of the prior art and FIG. 16B is a schematic top plan view of a user with the rollator of FIG. 1A illustrating a ground space required to operate the rollator.

FIG. 17 is a schematic left side elevation view of the rollator of FIG. 1A with the seat in a seated configuration and the handgrip frame in a frontward configuration in accordance with an embodiment wherein the rollator is used as a desk chair with a desk or a kitchen table.

FIG. 18 is a schematic left side elevation view of the rollator of FIG. 1A with the seat in a raised configuration in accordance with an embodiment wherein the rollator is used to give lateral and frontal support to a user working at a counter level work surface or in front of a kitchen/work counter/lavatory sink.

FIG. 19 is a schematic left side elevation view of the rollator of FIG. 1A with the seat in a seated configuration and the handgrip frame in a frontward configuration in accordance with an embodiment wherein the rollator is used in a transport chair mode involving a caregiver helping a user and their relative position towards each other.

FIG. 20 is a rear perspective view of the rollator of FIG. 1A with the seat in a seated configuration, the handgrip frame in a frontward configuration and with feet rests mounted in accessories receiving tubes.

FIG. 21 is a schematic right-side elevation view of the rollator of FIG. 1A with the seat in a raised configuration and the handgrip frame in a frontward configuration in accordance with an embodiment wherein the rollator is rolled above a toilet.

FIG. 22 is a rear perspective view of the rollator of FIG. 1A with the seat in a raised configuration and with an oxygen tank and tank support mounted to the accessory receiving tube.

FIG. 23 is a front perspective view of the rollator of FIG. 1A with the seat in a raised configuration and with a solute poll mounted to the accessory receiving tube.

FIG. 24 is a schematic front perspective view of the rollator shown in FIG. 1A having a walking re-education assembly in accordance with an embodiment mounted thereto;

FIG. 25 is a front elevation view of a wheel braking assembly including a gear disk and a wheel stopper in accordance with another embodiment wherein the gear disk includes a plurality of triangular-shaped teeth.

FIG. 26A is a rear perspective view of a rollator in accordance with an embodiment wherein the rollator is used with wheelchair wheels to ease propulsion when a user is in a seated position.

FIGS. 26B and 26C are respectively a left side elevation view and a front elevation view of the rollator of FIG. 26A.

FIG. 27A is a left side elevation view of the rollator of FIG. 26A, with a cover removed showing a wheel braking system.

FIG. 27B is an enlarged view of the wheel braking assembly of the rollator shown in FIG. 27A.

FIG. 28A is a side elevation view from outside of a section of the wheel braking assembly of FIG. 27B.

FIG. 28B is a side view from inside of a section of the wheel braking assembly of FIG. 27B.

FIG. 28C is an exploded view of the section of the wheel braking assembly of FIG. 28A.

It will be noted that throughout the appended drawings, like features are identified by like reference numerals.

### DETAILED DESCRIPTION

In the following description, the same numerical references refer to similar elements. Furthermore, for the sake of simplicity and clarity, namely so as to not unduly burden the figures with several references numbers, not all figures contain references to all the components and features, and references to some components and features may be found in only one figure, and components and features of the present disclosure which are illustrated in other figures can be easily inferred therefrom. The embodiments, geometrical configurations, materials mentioned and/or dimensions shown in the figures are optional, and are given for exemplification purposes only.

Moreover, although the embodiments of the rollator and corresponding parts thereof consist of certain geometrical configurations as explained and illustrated herein, not all of these components and geometries are essential and thus should not be taken in their restrictive sense. It is to be understood, as also apparent to a person skilled in the art, that other suitable components and cooperation thereinbetween, as well as other suitable geometrical configurations, may be used for the rollator, as will be briefly explained herein and as can be easily inferred herefrom by a person skilled in the art. Moreover, it will be appreciated that positional descriptions such as “above”, “below”, “left”, “right” and the like should, unless otherwise indicated, be taken in the context of the figures and should not be considered limiting.

Moreover, it will be appreciated that positional descriptions such as “above”, “below”, “forward”, “rearward” “left”, “right” and the like should, unless otherwise indicated, be taken in the context of the figures and correspond to the position and orientation of the rollator and corresponding parts, with the “front” corresponding to a position closer to a front portion of the rollator including a backrest and the “rear” corresponding to a position closer to a rear portion of the rollator, away from the backrest. Positional descriptions should not be considered limiting.

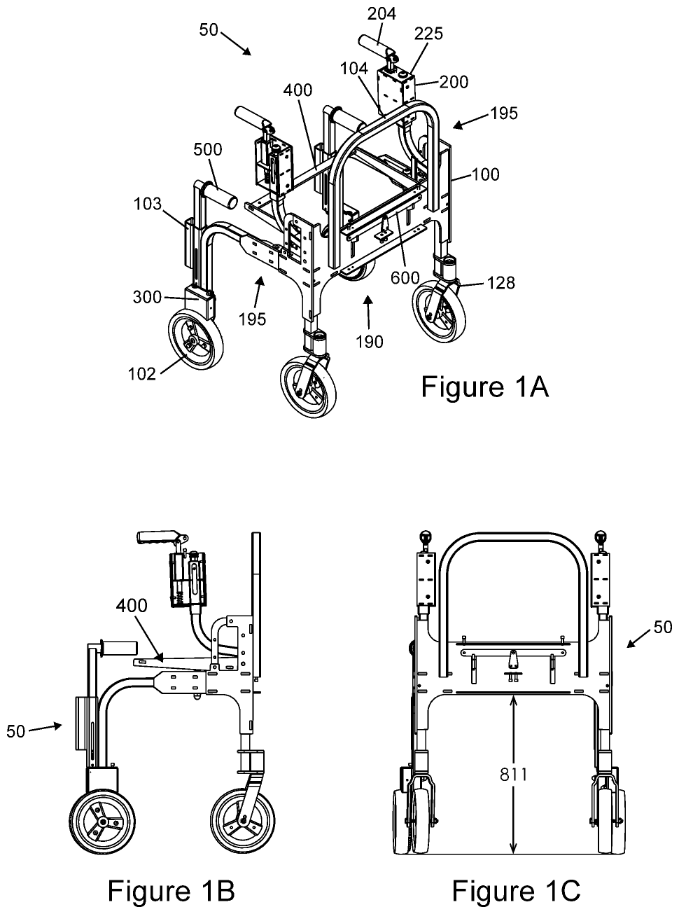

Referring to FIGS. 1A, 1B and 1C, there is shown an embodiment of a rollator 50 for mobility assistance to a user. The rollator 50 can be used as a rollator/walker, but it is appreciated that the rollator is not limited to this mode of operation. As further explained below, the rollator 50 can be used, among applications, as a transport chair, as a wheelchair, as an office wheeled chair and as a re-education tool.

The rollator 50 comprises a chassis 100 including a front portion 190 and two lateral side portions 195, each one of the lateral side portions 195 extending rearwardly from a respective end of the front portion 190. As will be described in more details below, the chassis 100 of the rollator 50 is designed to allow its use in most non-adapted environments such as public toilets, elevators, kitchen counter, office desks, etc.

In the embodiment shown, the lateral side portions 195 extend rearwardly from a respective end of the front portion 190 and substantially normal to the front portion 190 providing a good positioning and good front and lateral stability to the rollator user. By substantially normal, it is meant that an angle between one of the lateral side portions 195 and the front portion 190 is about 85° and about 110°.

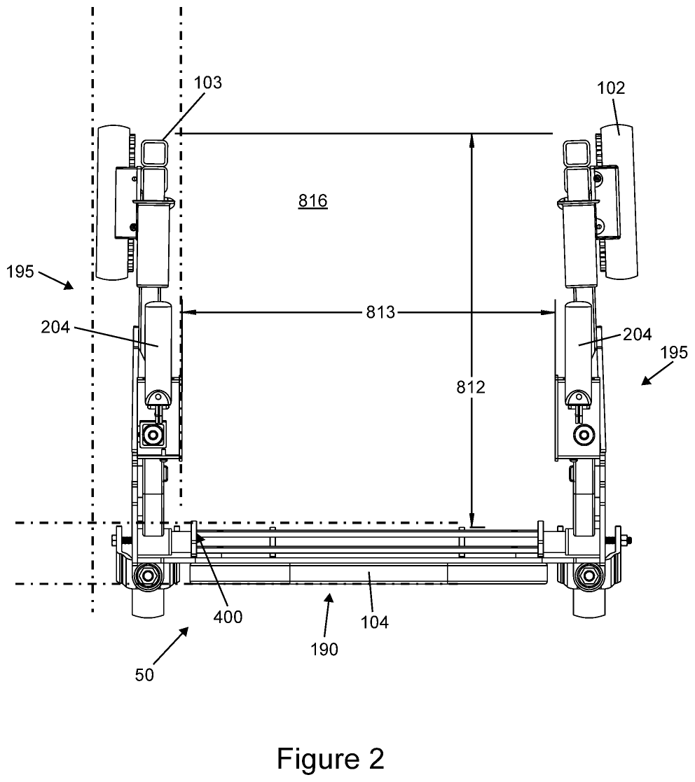

Referring now to FIG. 2, there is shown that the chassis 100 of the rollator 50 has a substantially U-shape (in a top plan view) that defines a seat/walker receiving area 816 (or operating area) and provides a completely cleared interior, i.e. which is unobstructed, that is, free of transversal members extending between the lateral side portions 195, and that allows a user to position himself inside of the chassis 100 giving lateral and front accessible handrail support, as will be described in more details below. The seat/walker area 816 is defined by a distance 813 between the two lateral side portions 195 of the chassis 100 and a distance 812 behind the front portion 190 of the chassis 100 (such as the front handrail 104) and a rear end of the two lateral side portions 195. The seat/walker area 816 allows to travel within the chassis 100 of the rollator 50, thereby providing improved lateral support and be more compacted when travelling in a limited space (for instance inside an elevator).

It is appreciated that, in an alternative embodiment, the shape and configuration of the chassis, including the front portion 190 and the lateral side portions 195, may differ from the embodiment shown in the accompanying figures.

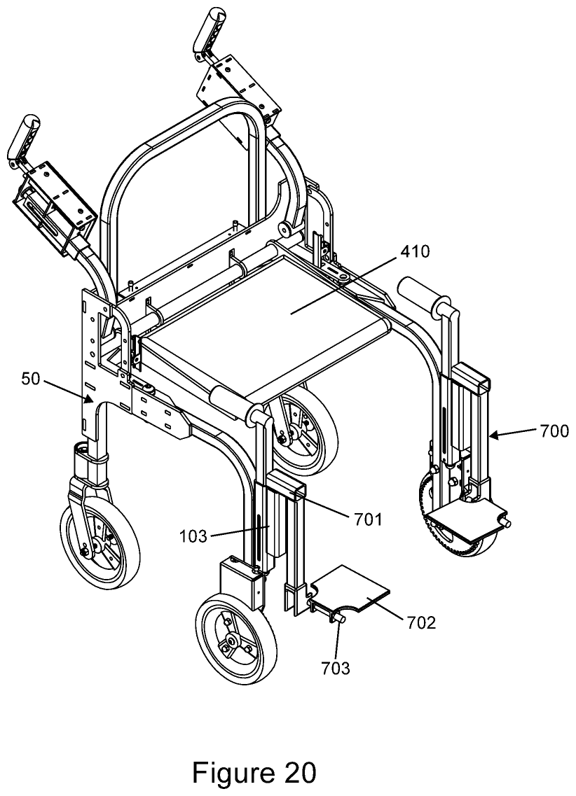

In addition to the U-shaped chassis 100, the rollator 50 includes, amongst others, a pair of primary handgrip assembly 265 provided with primary pivotable handgrips 204, at least one of accessory receiving tube 103 mounted to the chassis 100, a front handrail 104, a seat assembly 400 including a seat 410 (FIG. 20), two rear wheels 102, two front wheels 128, a wheel braking system 650 including wheel braking assemblies 300 and a wheel braking assembly controller 600, and a pair of secondary handgrips 500.

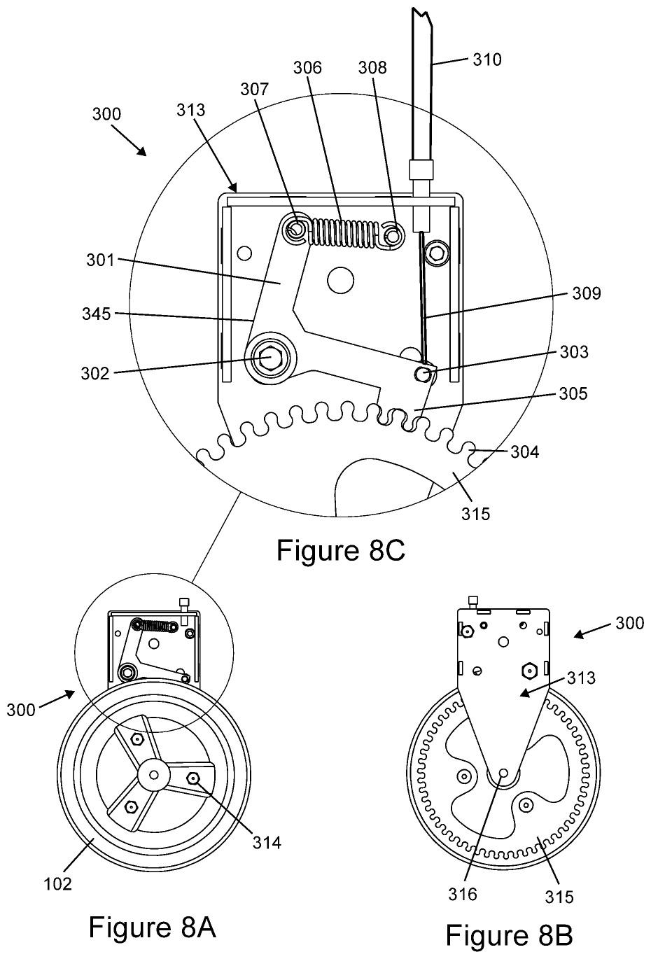

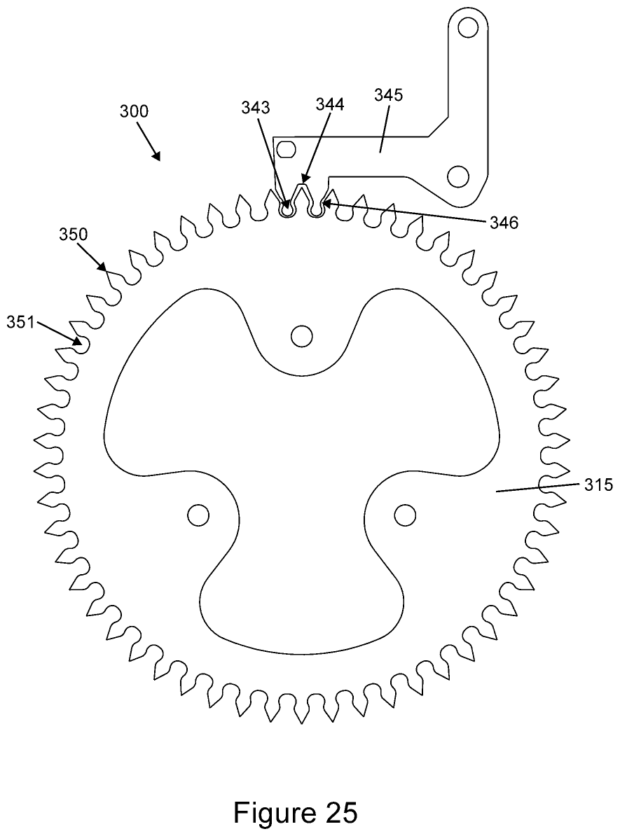

In an embodiment, swivel wheels 128 are mounted to the front portion 190 of the chassis 100 and fixed wheels 102 are mounted to rear ends of the lateral side portions 195 of the chassis 100. Each one of the wheel braking assemblies 300 includes a wheel stopper 345 (FIGS. 8 and 25). In an embodiment, the wheel stoppers 345 are engageable with a respective one of the fixed wheels 102 and configurable in a displacement configuration wherein they are disengaged from their respective wheel 102 to allow rotation thereof and a braking configuration wherein the respective wheel stopper 345 is engaged its respective fixed wheel 102 and prevent rotation thereof and, therefore, displacement of the rollator 50. The handgrips 204 of the primary handgrip assemblies 265 are mounted (directly or indirectly) to the chassis 100 and operatively connected to the wheel braking assemblies 300. The handgrips 204 are selectively configurable in a handgrip displacement configuration (i.e. lowered configuration) and in a handgrip braking configuration (which can be either the raised configuration or the lowered configuration, as will be described in more details below). Configuration of at least one of the handgrips 204 in the wheel braking configuration configures the two wheel stoppers 345 in the braking configuration wherein they are engaged with their respective one of the fixed wheels 102. Therefore, configuration of both handgrips 204 in the handgrip displacement configuration, with a pressure applied thereon below a handgrip pressure threshold) is required to have the two wheel stoppers 345 disengaged from the fixed wheels 102 and to allow displacement of the rollator 50.

It is appreciated that the wheel braking assemblies 300 can be engaged with the front wheels instead of the rear wheels or with both the front and rear wheels. It is also appreciated that the front and rear wheels can be either swivel or fixed wheels and that the wheel braking assemblies can be engaged with either swivel or fixed wheels.

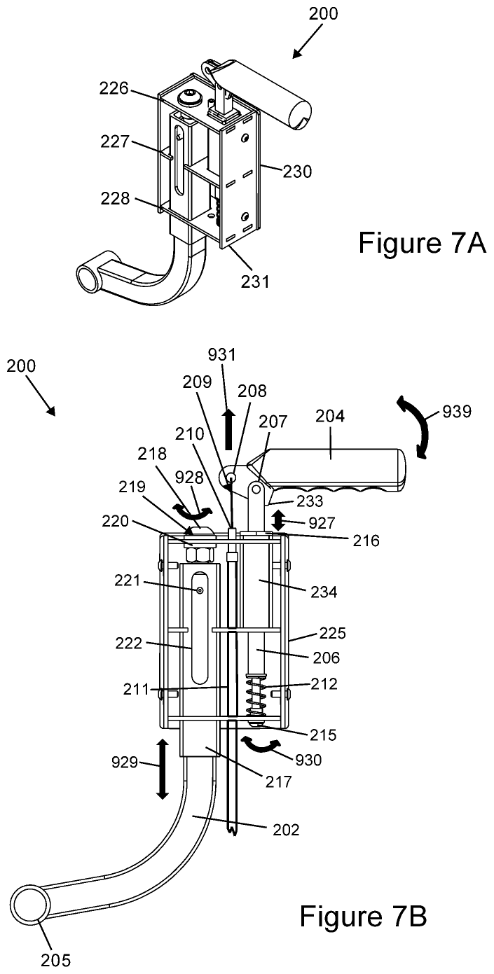

In an embodiment, with reference to FIGS. 7A and 7B, each one of the handgrip assemblies 200 includes a pivotable handgrip 204 which is operatively connected to the wheel braking assembly(ies) 300. As mentioned above, each one of the pivotable handgrips 204 is selectively configurable in: a raised configuration and a lowered configuration, wherein the handgrip extends substantially horizontally. The raised configuration is a wheel braking configuration, wherein at least one of the wheel braking assemblies 300 is engaged with at least one of the wheels and prevent rotation thereof. If both handgrips are configured in the lowered configuration simultaneously and the pressure applied thereon is below a handgrip pressure threshold, the wheel braking assemblies are disengaged from the wheels. However, if at least one of the handgrips is configured in the lowered configuration and the pressure applied thereon is equal to or greater that a handgrip pressure threshold, the wheel braking assemblies are configured in the braking configuration, wherein they are engaged with the wheels to prevent rotation thereof.

In an embodiment, the pivotable handgrips 204 are configured in the raised configuration when no pressure is applied thereon, i.e. they are biased towards the raised configuration.

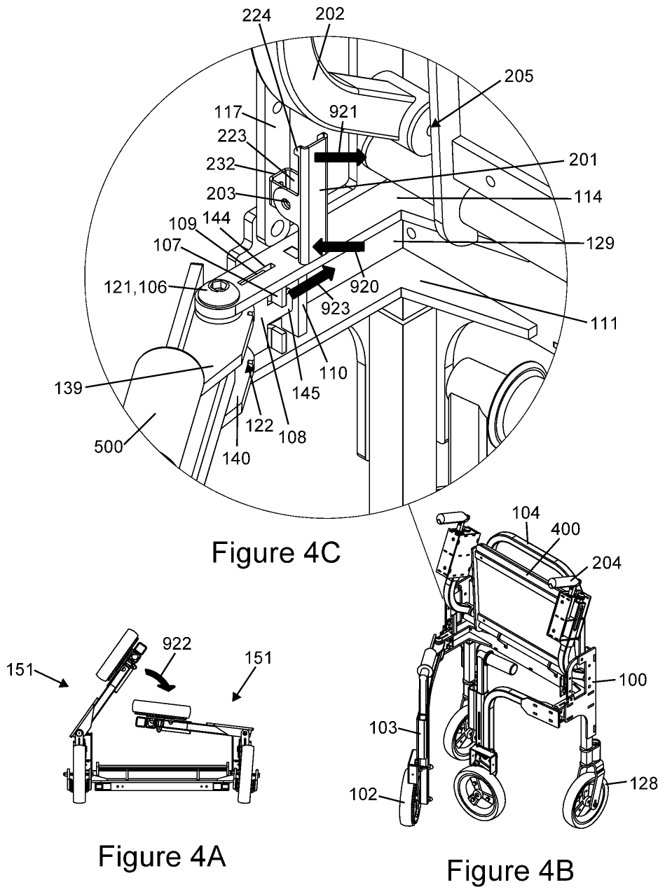

In an embodiment, with reference to FIGS. 1A, 1B, 4A and 4B, the seat 410, mounted to seat frame 401, of the seat assembly 400 (only the seat frame is shown) is pivotally mounted to the chassis 100 and configurable between a raised configuration and a seated configuration wherein the seat 410 extends substantially horizontally in the seat/walker receiving area 816. In an embodiment, the seat assembly 400 is operatively connected to the wheel braking assembly(ies) 300 and, when the seat 410 is configured in the seated configuration, the wheel braking assemblies 300 are configured in the displacement configuration, i.e. disengaged from the rear wheels 102.

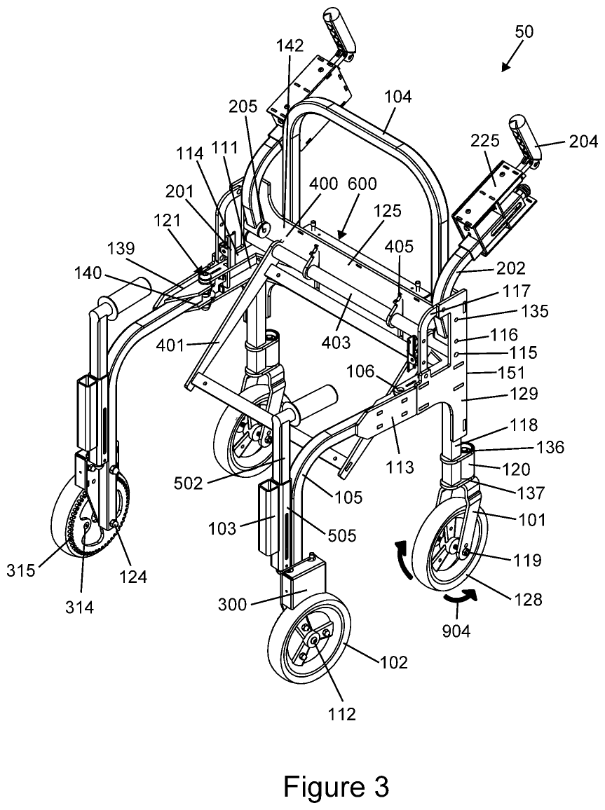

In an embodiment, with reference to FIG. 3, when the seat/walker receiving area 816 is unobstructed when the seat 410 is configured in the raised configuration.

In an embodiment, with reference to FIGS. 1B and 3, the primary handgrip assembly 265 includes at least one handgrip frame 202 (or swing arms) pivotally mounted to the chassis 100. In the embodiment shown, the handgrip frame 202 includes two lateral side portions 202A, 202B extending substantially in a same plane than a respective one of the lateral side portions 195 of the chassis 100. Each one of the lateral side portions 202A, 202B of the handgrip frame 202 includes one of the handgrips 204 mounted thereto. The handgrip frame 202 is configurable in an operative configuration wherein the handgrips 204 are located rearwardly of the front portion 190 of the chassis 100 and a frontward configuration wherein the handgrips 204 are located forwardly of the front portion 190 of the chassis 100.

In the embodiment shown, the lateral side portions 202A, 202B are independently pivotable in the operative configuration and the frontward configuration. However, in an alternative embodiment, it is appreciated that the handgrip assembly can include a single handgrip frame wherein the handgrips mounted to the lateral side portions 202A, 202B are mechanically connected and simultaneously pivotable in the operative configuration and the frontward configuration.

In an embodiment, with reference to FIG. 1A, the rollator 50 also includes a pair of secondary handgrips 500 mounted to a respective one of the lateral side portions 195 of the chassis 100 and adjacent to a rear end thereof. The secondary handgrips 500 extend below the primary handgrips 204 mounted to the pivotable handgrip frame 202 in the operative configuration thereof.

In an embodiment, with reference to FIG. 3, the chassis 100 includes a plurality of vertically-extending members, such as legs 118 and axle tube 105, with at least some of the vertically-extending members including at least two telescopically-engaged segments to adjust a height of the chassis 100.

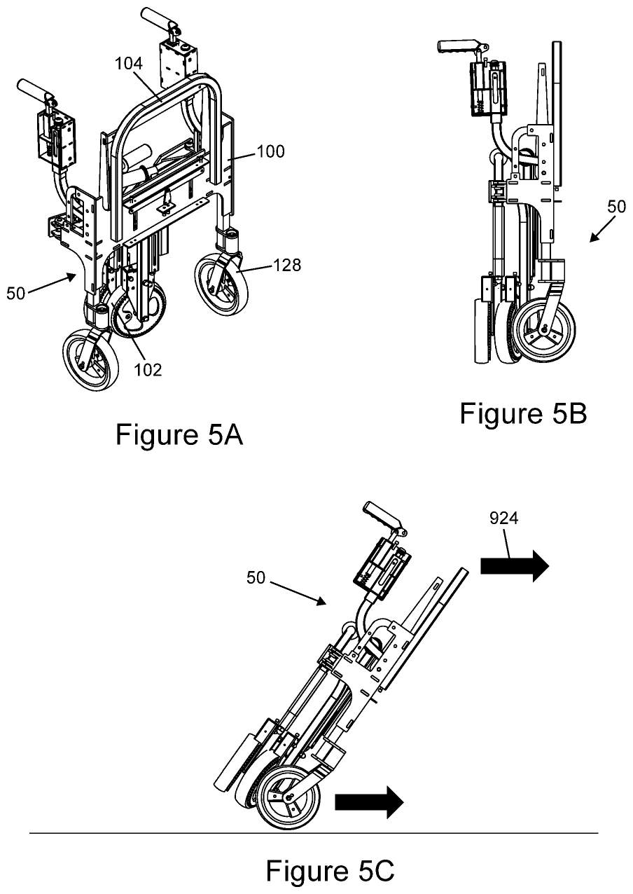

In an embodiment, with reference to FIGS. 4A to 4C and 5A and 5B, the two lateral side portions 195 are pivotally mounted to a respective end of the front portion 190 to be configurable in an operative configuration wherein they are spaced-apart from one another and extend substantially parallel to each other to define the seat/walker receiving area 816 inbetween and a compacted configuration wherein they are superposed to each other and extend substantially parallel to the front portion 190. In an embodiment, in the operative configuration, the lateral side portions 195 extend substantially normal to the front portion 190.

In an embodiment, with reference to FIGS. 8A to 8C and 25, each one of the wheel braking assemblies 300 includes a gear disk 315 secured to a respective one of the wheels of the rollator 50. The gear disk 315 has a plurality of triangular-shaped teeth 315 extending peripherally. The wheel stopper 345 of the wheel braking assembly 300 is engageable with the respective gear disk 315 and configurable in the displacement configuration wherein it is disengaged from the respective gear disk 315 to allow rotation of the corresponding wheel and a braking configuration wherein it is engaged with the respective gear disk 315 to prevent rotation of the corresponding wheel. In an embodiment, the gear disk 315 also includes a plurality of rounded recesses 351 with adjacent ones of the triangular-shaped teeth 350 being separated by one of the rounded recesses defined inbetween.

In an embodiment, with reference to FIGS. 1A and 1B, the chassis 100 has at least one accessory receiving tube 103 mounted thereto. It can be secured to the lateral side portions 195 and/or the front portion 190. In the embodiment shown, the chassis 100 includes two pairs of accessory receiving tubes 103, each one of the pairs being mounted on a respective lateral side portions 195 of the chassis 100, rearwardly thereof. It is appreciated that the number, the configuration, and the position of the accessory receiving tube(s) can vary from the embodiment shown.

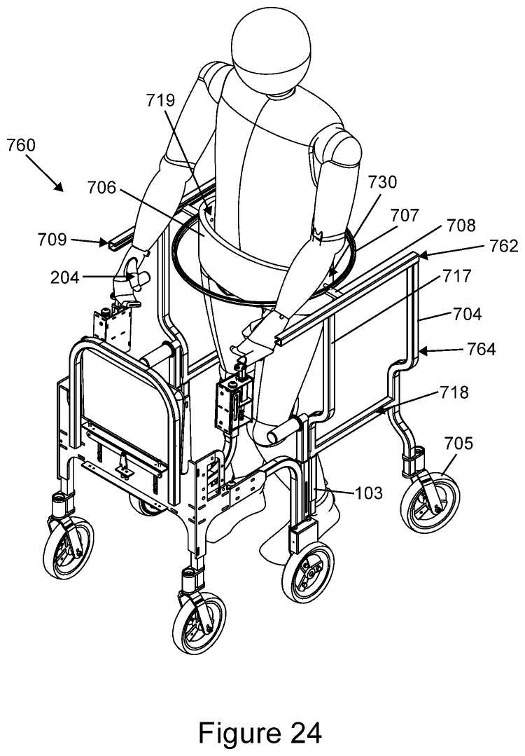

In an embodiment, with reference to FIG. 24, the rollator 50 includes a walking re-education assembly 760 including a frame 762 removably engageable with the chassis 100 and a ring 707. The frame 762 has two lateral side portions 764 spaced-apart from one another and extending substantially parallel to a respective one of the lateral side portions 195 of the chassis 100 when engaged therewith. The ring 707 can be attached to the two lateral side portions 764 of the frame 762 and can extend therebetween. The ring 707 is configured to partially support a weight of the user during a rehabilitation process.

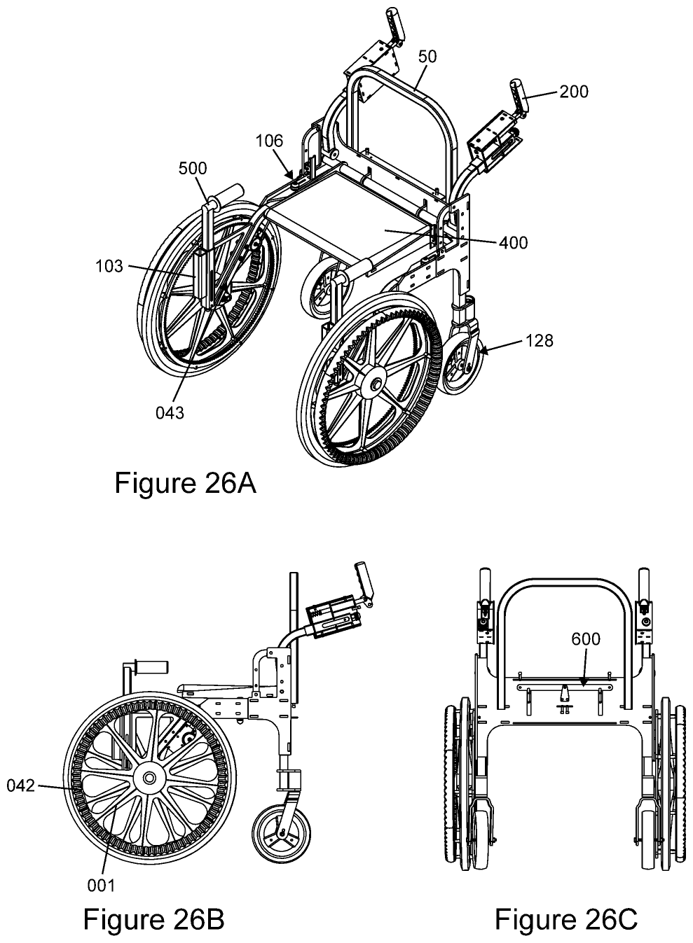

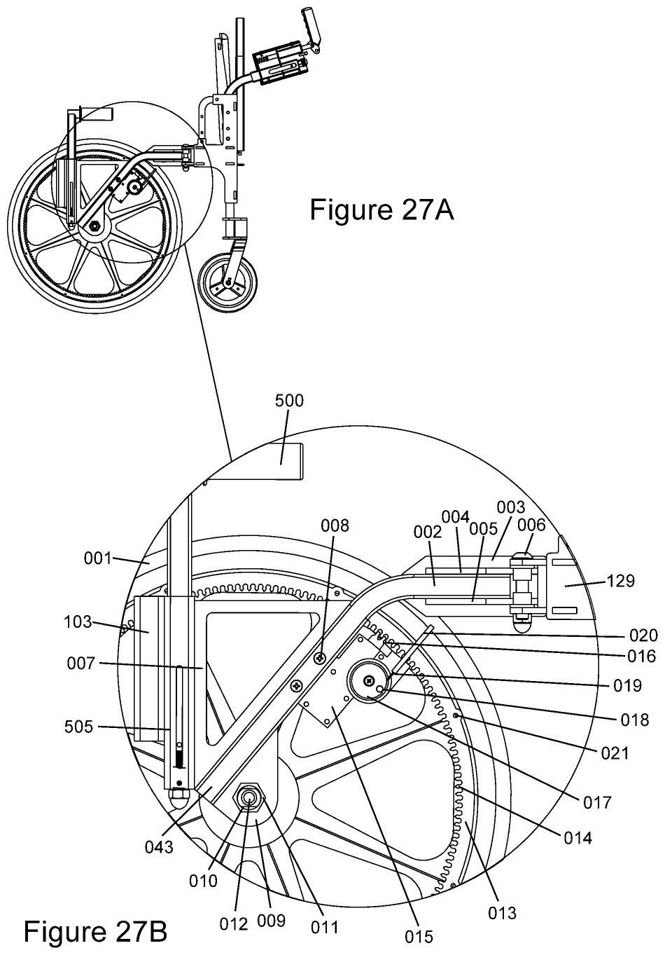

In an embodiment, with reference to FIGS. 26 and 27, the rollator 50 can be provided with at least two sets of rear wheels, each one of the sets of rear wheels including at least a pair of rear wheels and being characterized by a different wheel diameter, the rear wheels being detachably and rotatably securable to the lateral side portions 195 of the chassis 100 and each one of the sets being selectively securable to the lateral side portions 195 of the chassis 100.

In the embodiment shown, each one of the lateral side portions 195 is substantially L-shaped with a horizontally-extending section pivotally connected to the front portion 190, as will be described in more details below, and a downwardly extending section configured to receive one of the fixed wheels 102 and a respective one of the wheel braking assemblies 300 at a lower end thereof. In the embodiment shown, each one of the downwardly extending sections of the lateral side portions 195 has two accessory receiving tubes 103 mounted rearwardly thereto. In the embodiment shown, the accessory receiving tubes 103 are embodiment by two square tubings that allow the user to install accessories like the secondary handgrips 500, which will be described in more details below. The secondary handgrips 500 can be used to stand up or sit down of the rest seat 410. In an embodiment, the secondary handgrips 500 can be adjustable in height, as described in more details below.

In the embodiment shown, the front portion 190 includes a combination of one or several horizontally and vertically extending structural members. It also includes two rearward extensions 151, one on each side, each one being connected to a respective one of the lateral side portion 195. The front portion 190 also includes a front plate 125 extending between the two rearward extensions 151 and two lateral plates 129. In an embodiment, the front plate 125 is substantially normal to each one of the lateral plates 129. The front portion 190 of the chassis 100 also includes L-shaped reinforcement brackets 117, each one being secured to a respective one of the lateral chassis plate 129.

As shown in FIG. 4C, the front portion 190 of the chassis 100 also includes upper and lower reinforcement angle plates 114, 111 secured to the lateral plates 129 and the front plate 125, in the inside corners of the U-shaped chassis 100. The upper and lower reinforcement angle plates 114, 111 increase the rigidity of the chassis 100. For instance and without being limitative, the upper and lower reinforcement angle plates 114, 111 can be secured to the lateral plates 129 and the front plate 125 by welding.

At a rear end 116 thereof, the upper and lower reinforcement angle plates 114, 111 have through holes defined therein. The holes defined in the upper reinforcement angle plate 114 are in register with the holes defined in the lower reinforcement angle plate 111 in a manner such that the lateral side portions 195 of the chassis 100 are pivotally connected to the upper and lower reinforcement angle plates 114, 111 of the front portion 190 of the chassis 100 through these through holes, as will be described in more details below with reference to FIGS. 4A to 4C and 5A to 5C.

The vertically extending structural members of the front portion 190 includes two legs 118 extending downwardly from opposed lateral ends of the front portion 190. A respective one of the swivel wheels 128 is rotatably mounted to a lower end of each one of the legs 118. Each one of the lateral plates 129 is also secured to a respective one of the legs 118.

It is appreciated that the shape and configuration of the chassis 100, including the front and lateral side portions 190, 195 and their components may vary from the embodiment shown. For instance and without being limitative, the front and lateral side portions 190, 195 of the chassis 100 are conceived with square cross-section tubing but it is appreciated that, in alternative implementations, tubing of any cross-section can be used.

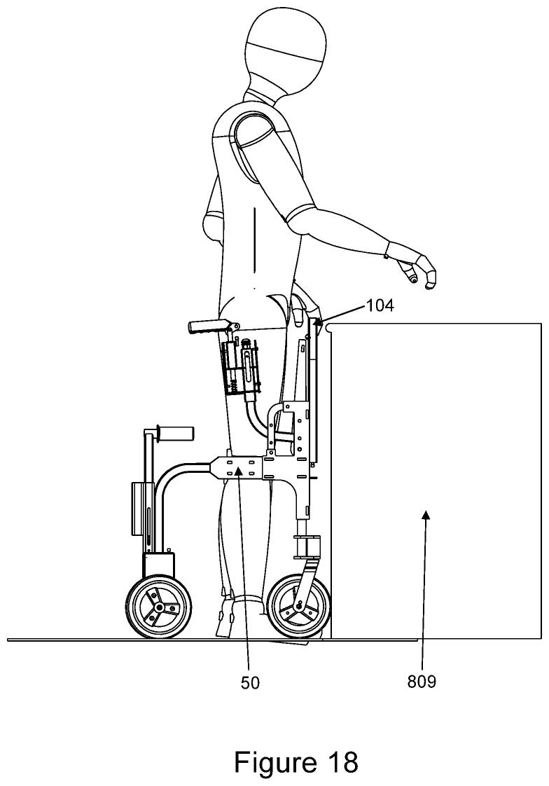

The front portion 190 also includes a substantially U-shaped handrail 104. In an embodiment, the U-shaped handrail 104 is fixed to the chassis 100, i.e. it is not pivotally mounted, and, more particularly, to a front face of the front plate 125. In the embodiment shown, the handrail 104 is made of a square cross-section tubing but it is appreciated that the shape and the configuration of the tubing and the handrail 104 may vary. In an embodiment, the front handrail 104 can be secured through mechanical fasteners, such as bolts, by welding or by any other suitable technique. In an embodiment, with reference to FIG. 18, a height of a horizontally-extending section of the handrail 104, with respect to the ground, will exceed the height of a regular kitchen counter 809 by a distance sufficient to exceed the countertop in a way to leave sufficient space for the user fingers to grip the handrail 104. Thus, the handrail 104 can be used as a frontal support for the user while working at a kitchen or any type of counter 809 (FIG. 18).

Furthermore, the U-shaped handrail 104 can act as a backrest. In an embodiment, it can be covered with a lining material (not shown), such as and without being limitative, soft fabric or plastic material attached on the two vertical sections of the U-shaped handrail 104. In an embodiment, the lining material could be made of transparent vinyl material to reduce potential blinder. In an embodiment, the backrest can be a cushioned backrest mounted to the handrail 104. It is appreciated that, in an alternative embodiment, the shape and configuration of the handrail 104 may differ from the embodiment shown in the accompanying figures. Furthermore, the front handrail 104 can be adjustable in height or pivotally mounted to modify its inclination with respect to the seat 410.

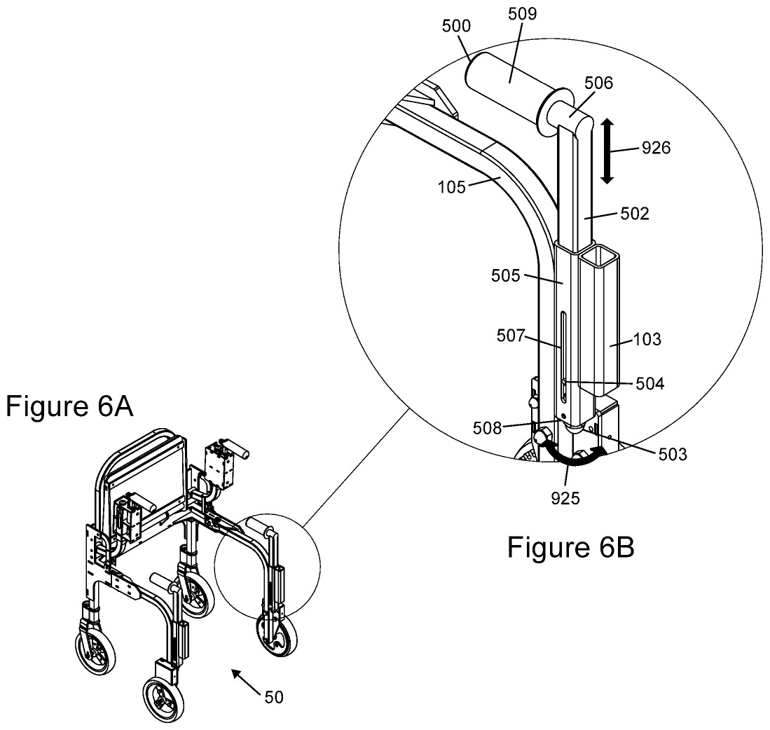

In the embodiment shown, the rollator 50 also includes a seat assembly 400 mounted to the chassis 100 and, more particularly, pivotally mounted to the front portion 190 of the chassis 100. As will be described in more details below, the seat assembly 400 includes a seat 410 configurable between a raised configuration (FIGS. 2 and 6A) and a seated configuration (see FIGS. 1A and 1B). In the raised configuration, the seat 410 is abutted against the handrail 104, i.e. it extends substantially vertically, freeing the seat/walker receiving area 816. The seat 410 can be configured in the raised configuration when using the rollator 50 in the walking mode of transportation. In the seated configuration, the seat 410 extends substantially horizontally in the seat/walker receiving area 816. The seat 410 can be selectively configured in the seated configuration when using the rollator 50 in the seated mode and in the raised configuration when using the rollator in a walking mode.

In the embodiment shown, the seat assembly 400 is pivotally mounted to the lateral plates 129 at rear seat pivot attachment points 115. It is appreciated that the position of the rear seat pivot attachment points 115 can vary on the chassis 100 and on the lateral plates 129.

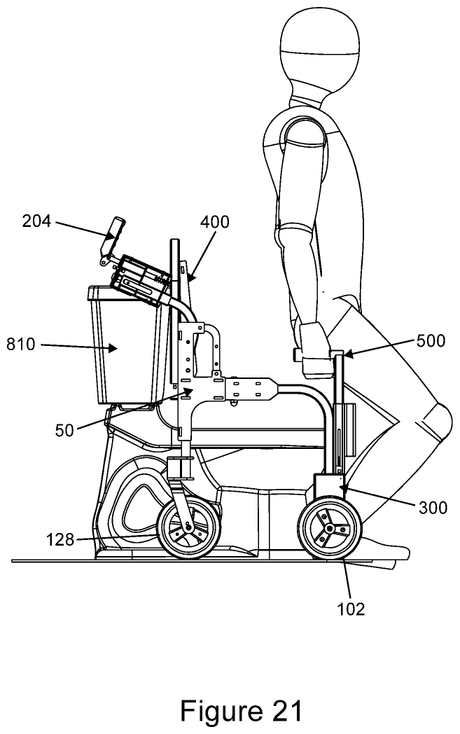

When configured in the seated configuration, the seat 410 is spaced apart from the ground by a chassis distance 811. In an embodiment, the chassis distance 811 should be sufficient so that the rollator 50 can be rolled over a standard public toilet, as shown in FIG. 21.

In the embodiment shown, the wheels mounted to lateral side portions 195 are fixed wheels while the wheels mounted to the front portion 190 are swivel wheels. However, it is appreciated that, in alternative embodiments, the type of wheels mounted to the front and lateral side portions 190, 195 can vary from the embodiment shown. For instance and without being limitative, the swivel wheels can be mounted to the lateral side portions 195 while the fixed wheels can be mounted to the front portion 190. Furthermore, the four wheels can be either swivel or fixed wheels.

Referring to FIG. 3, there is shown that the swivel wheels 128 are mounted to the legs 118 at the front portion 190 of the chassis through upper and lower swivel wheel bearing barrel attachment brackets 136, 137. Each one of the swivel wheel assemblies also includes a swivel wheel bearing barrel 120 and a wheel fork 101. The swivel wheel bearing barrel 120 is mounted to the leg 118 through the upper and lower swivel wheel bearing barrel attachment brackets 136, 137. The swivel wheel fork 101 is mounted to the rollator chassis 100 via its pivoting shaft which is connected coaxially with the swivel wheel bearing barrel 120 with two roller bearings (not shown) one located at the top end of the swivel wheel bearing barrel 120 and one located at the bottom. The swivel wheel 128 is mounted to the swivel wheel fork axle 101 through an axle bolt 119 on which the swivel wheel 128 can rotate freely about its rotation axis. The swivel wheel fork 101 allow the swivel wheel 128 to pivot to the left or to the right as shown by arrows 904.

In the embodiment shown, the rollator 50 also includes two wheel braking assemblies 300, each one having a wheel stopper 245 engageable with a respective one of the rear and fixed wheels 102 and configurable in a displacement configuration allowing rotation of the wheels 102 and a braking configuration wherein the wheel braking assembly 300 is engaged with the respective one of wheels 102 and prevent rotation thereof. In the embodiment shown, each one of the wheel braking assemblies 300 is engageable with a respective one of the fixed wheels 102 mounted to the lateral side portions 195. However, it is appreciated that, in alternative embodiments, the wheel braking assemblies 300 can be engaged with the wheels mounted to the lateral side portions 195 and/or the front portion 190, which can be either swivel or fixed wheels, any combination thereof being possible. As will be described in more details below, in the embodiment shown, each one of the wheel braking assemblies 300 includes gear-type brake disks 315.

In the embodiment shown, each one of the fixed wheels 102 can be engaged by a respective one of the wheel braking assemblies 300.

In the embodiment shown, the rollator 50 also includes two primary handgrip assemblies 200, one for each lateral side frame 195. Each primary handgrip assembly 200 includes a handgrip frame 202 pivotally mounted to the chassis 100, a handgrip control box 225 mounted to the handgrip frame 202, and a handgrip 204 mounted to the handgrip control box 225.

In the embodiment shown, each of the handgrip frame 202 is pivotally mounted to a respective one of the rearward extensions 151 of the front portion 190, inwardly thereof. More particularly, each of the handgrip frame 202 is pivotally mounted to a respective one of the lateral plates 129 at handgrip pivot attachment point 116, located close to a front end of the respective lateral plate 129. In the embodiment shown, the handgrip frames 202 are substantially L-shaped with a first end pivotally mounted to the chassis 100 and the handgrip control box 235 being mounted close to the opposed end.

Furthermore, in some implementations, the lateral plates 129 can be provided with more than one handgrip pivot attachment point. In the embodiment shown in FIG. 3, the lateral plates 129 are provided with a second handgrip pivot attachment point 135. The handgrip frame 202 can be pivotally mounted to the chassis 100 through this second handgrip pivot attachment point 135 if the rollator user is tall and the handgrip pivot attachment points 116 are too low to provide an ergonomic handgrip position to the user.

The handgrip frames 202 can be configured into an operative configuration (shown in FIGS. 1A to 1C) wherein the handgrips 204 are located rearwardly of the front portion 190 and can be used to receive the hands of the rollator user in a walking mode and partially support its weight. The handgrip frames 202 can also be configured into a forward configuration (shown in FIG. 3) wherein the handgrips 204 are located forwardly of the front portion 190 of the chassis 100. For instance, the handgrip assemblies 200 can be configured in the forward configuration when the rollator is used as a transport chair to provide more freedom laterally to a user seated on the rollator seat 410. In FIG. 3, the seat 410 is configured in an exaggerated low position for clarity purposes.

In the embodiment shown, each one of the handgrip assemblies 200 includes its own handgrip frame 202 and are therefore independently pivotable between the operative configuration and the frontward configuration. However, it is appreciated that, in an alternative embodiment, the handgrip frame(s) 202 of both handgrip assemblies 200 can be connected or can be single piece and the handgrips 204 can pivot simultaneously in either the operative configuration and the forward configuration.

As will be described in more details below, when no pressure is applied thereon, the handgrips 204 are biased into a raised configuration. The handgrip control boxes 225 include an handgrip configuration detection assembly that is configured to detect the configuration of the respective one of handgrips 204 and, more particularly, whether the handgrip 204 has been depressed into a lowered configuration and if the pressure applied thereof is below or greater than the handgrip pressure threshold.

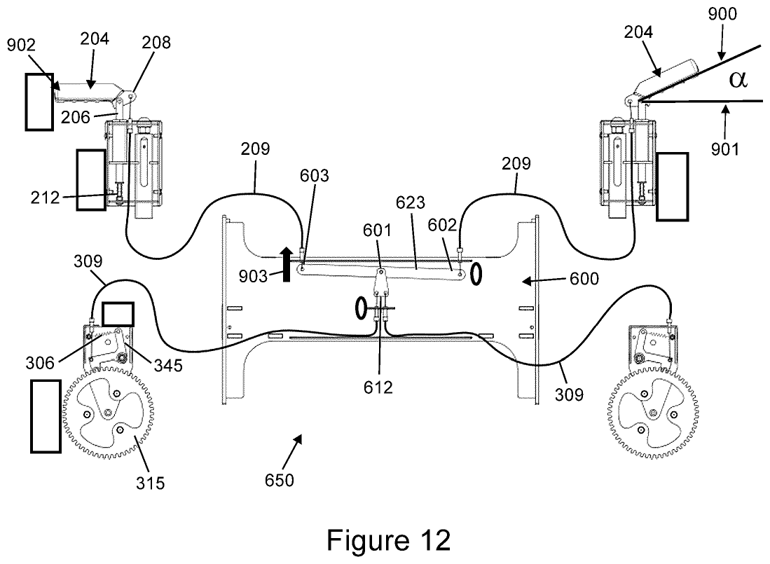

As will be described in more details below, the handgrip control boxes 225 are operatively connected to the wheel braking system 650 (FIGS. 12 to 14) and, more particularly, to the wheel braking assemblies 300. In an embodiment shown, the handgrip control boxes 225 are mechanically connected to the wheel braking assemblies 300 through control cables, such as and without being limitative, stainless multi-strand cables contained into nylon sheath.

The wheel braking system 650 also includes a wheel braking assembly controller (or hazard control mechanism) 600, which is mounted to the front portion 190 of the chassis 100 and, more particularly, to a front face of the front plate 125. The handgrip control boxes 225 are mechanically connected to the wheel braking assemblies 300 via the wheel braking assembly controller 600 and through the control cables.

Referring now to FIGS. 4A to 4C and FIGS. 5A to 5C, there is shown that the lateral side portions 195 of the chassis 100 are pivotally mounted to the front portion 190 through vertically-extending chassis hinges 106. Therefore, the lateral side portions 195 can be folded against the front portion 190 in a compacted configuration (or folded configuration) as shown in FIGS. 5A to 5C, which can be convenient for storage and transport. The chassis 100 also includes chassis pivoting lock assemblies for locking the vertically-extending hinges 106 in an operative configuration (see FIGS. 1A to 1C) wherein the lateral side portions 195 are spaced-apart from one another and extend substantially parallel to each other to define the seat/walker receiving area 816 inbetween.

In the compacted configuration, the lateral side portions 195 of the chassis 100 are folded over and extend substantially parallel to the front plate 125 of the front portion 190.

As shown in FIG. 5B, in the compacted configuration, the rollator 50 remains vertically stable with its four wheels 102, 128 contacting the ground, with the two fixed wheels 102 being engaged by their respective wheel braking assembly 300 in the braking configuration. Therefore, the rollator 50 remains stationary. As shown in FIG. 5C, in the compacted configuration, the rollator 50 can be tilted forwardly and pulled by the front handrail 104, as shown by arrows 924, to be rolled towards another location. In the forwardly tilted configuration, only the swivel wheels 128 contact the ground (i.e. the fixed wheels 102, which are engaged by their respective wheel braking assemblies 300 in the braking configuration, are spaced-apart from the ground). Therefore, the forwardly tilted rollator 50 can be freely displaced.

As mentioned above, the rollator 50 is equipped with the wheel braking assembly controller 600, acting as a fall detection system that detects if a person or user is about to fall or lose balance and will react accordingly by configuring the wheel braking assemblies into the braking configuration. The wheel braking assembly controller 600 is operatively connected to the handgrip assemblies 265 and the wheel braking assemblies 300. The handgrip assemblies 265 are sensing the motion behavior of the user's hands and transmit signals of the detected motion to the wheel braking assembly controller 600. Based on predetermined control logic, the wheel braking assembly controller 600 transmits signals to the wheel braking assemblies 300 in a manner such that the wheels braking assemblies 300 are configured either in the displacement configuration or the braking configuration.

The wheel braking assembly controller 600 is also operatively connected to the seat assembly 400. In an embodiment, when the seat 410 is configured in the seated configuration with the user seated thereon (i.e. with a pressure applied is equal to or greater than a seat pressure threshold), the wheel braking assembly controller 600 will configure the wheel braking assemblies 300 into the displacement configuration. The wheel braking assembly controller 600 will also automatically configure the wheel braking assemblies 300 in the braking configuration if the user attempts to stand up (i.e. if the pressure applied on the seat 410 is below the seat pressure threshold).

The rollator 50 can be manufactured and assembled using aluminium laser cut plates and aluminium square or rounded extrusion profiles, welded or attached together with the use of appropriate mechanical fasteners such as bolts, screws or nuts. It is understood that other suitable material can be used (in replacement or in combination with aluminium and aluminium alloys) including and without being limitative plastics, steel, and the like. For instance, the rear wheels 102 shown in the accompanying figures include an aluminium gear disk 315 bolted on a currently available wheelchair wheel but could also be cast within the wheel to form a single wheel of plastic. Similarly, the front plate can be made of a single aluminium casting in order to lower manufacturing costs but it is appreciated that other materials and configuration are included.

Referring now to FIG. 3, there is shown that the seat assembly 400 includes a pivoting tube 403 mounted at attachment points 115 to mount the seat frame 401 to the chassis 100 and, more particularly, to the front portion 190 of the chassis 100. The seat frame 401 pivots with the pivoting tube 403 between multiple configurations including the raised configuration and the seated configuration. In the embodiment shown, the seat frame 401 is secured to the pivoting tube 403. However, it is appreciated that, in an alternative embodiment, the seat frame 401 can be pivotally mounted to the pivoting tube 403. In the embodiment shown, the pivot attachment points 115 are located on a respective one of the lateral plates 129. The seat 410, shown in FIG. 20, can be mounted to the seat frame 401 (including two lateral frame structural members, a front transverse structural member and a back transverse structural member). As it is appreciated, the seat 410 can be a cushioned seat, a rigid-type of seat, a hammock-type of seat or any other suitable seat. Thus, the seat 410 is mounted between each of the lateral side portions 195 of the chassis 100.

As will be described in more details below, the pivoting tube 403 is provided with a pair of brake lifters 405 secured thereto. The brake lifters 405 are partially inserted in a pair of vertical lifter openings 622 defined the front plate 125. The brake lifters 405 are mounted at an equal distance with respect to a respective lateral frame portion 195.

In the embodiment show, the seat frame 401 is provided with a flat horizontal extension 404 at its respective front end which is designed to move within a set of vertical openings 142 defined in the front plate 125 of the chassis 100. The flat horizontal extension 404 is configured to abut against a respective upper surface of the vertical openings in a desired seat assembly configuration, therefore limiting the pivoting movement of the seat assembly 400 to a desired range of configurations. For example, it may be desired to prevent the seat assembly 400 from rotating towards the ground, i.e. at a negative angle with respect to a plane parallel to the ground.

In an embodiment, the seat assembly 400 can be adjustable in height in a manner such that it is possible for a rollator user to adjust the seat assembly height with respect to a ground surface according to his height for further comfort.

It is appreciated that the configuration and the configuration of the seat assembly 400 can vary from the embodiment described above with reference to the accompanying figures.

Still referring to FIG. 3, there is shown that the rollator 50 includes a pair of handgrip assemblies 265, each one including a handgrip frame 202 (or swing arm), a handgrip 204 mounted to the respective handgrip frame 202, and a handgrip control box 225. The handgrip frames 202 are pivotally mounted to the front portion 190 of the chassis 100 in a manner that each one can be pivoted independently of the other. In other words, one of the handgrip frame 202 can be positioned in a frontward configuration with its handgrip 204 located forwardly of the front portion 190 of the chassis 100 whereas the other handgrip frame 202 can be positioned in an operative configuration, or rearward configuration, wherein its handgrip 204 is located rearwardly of the front portion 190 of the chassis 100. However, it is appreciated that, in an alternative embodiment, the handgrip frames of the handgrip assemblies 265 can be mechanically connected or single piece in a manner such they pivot simultaneously.

In the embodiment shown, the handgrip frames 202 are made of square tubes and are substantially L-shaped. A lower end of each handgrip frame 202 is mounted to a cylindrical member 205 having a rotation axis parallel to the pivoting tube 203 of the seat assembly 400 and positioned above the pivoting tube 203. The cylindrical member 205 acts as a pivot allowing the frontward and rearward configurations of the handgrip frames 202. The lateral plates 129 include swing arm pivot holes 116, 135 to mount each handgrip frame 202 to the chassis 100 using a pivot bolt. In the embodiment shown, the chassis 100 is thus designed to provide two configurations for the height of the handgrip frames 202 by selecting one of the two swing arm pivot holes 116, 135, with the pivot attachment hole 116 being positioned below the pivot attachment hole 135. Therefore, mounting the handgrip assemblies 265 to the pivot attachment holes 135 can be more ergonomic for taller users.

It is appreciated that the shape and the configuration of the handgrip assemblies 200 including the handgrip frames 202 can vary from the embodiment described above with reference to the accompanying figures.

Referring now to FIG. 4, there is shown an embodiment of a locking mechanism of the handgrip assembly 265. The handgrip locking mechanism is designed to prevent unwanted or inappropriate pivoting movement of the handgrip frame 202. The handgrip locking mechanism includes a swing arm locking lever 201, which is a substantially U-shaped lever with a claw shaped extension 224 at one end that secures the handgrip frame 202 in the rearward configuration. The swing arm locking lever 201 includes a central extension including two pivot attachment holes 203. The swing arm locking lever 201 is mounted to the front portion 190 of the chassis 100 and, more particular to a U-shaped mounting bracket 232 secured to the L-shaped reinforcement bracket 117 through a pivot bolt (not shown). The locking mechanism also includes a rotation spring 223 mounted to a pivot bolt, coaxially with pivot attachment holes 203, and inside of the U-shaped mounting bracket 232. The spring 223 biases the swing arm locking lever 201 into a closed configuration where it prevents the handgrip frame 202 from pivoting by applying a constant closing pressure. To unlock the handgrip frame 202 and allow pivoting thereof, the rollator user has to apply pressure 920 on the lower part of the swing arm locking lever 201 which results in the upper part of the swing arm locking lever 201 to be displaced horizontally 921 around pivot attachment hole 203, thereby unlocking the handgrip frame 202. Once unlocked, the handgrip frame can be pivoted forwardly into the frontward configuration, which corresponds to the transport chair mode.

Referring back to FIG. 3, there is shown that each one of the lateral side portions 195 of the chassis 100 includes a substantially L-shape axle tube 105 and a lateral plate 113 having two spaced-apart rear hinge plates 139, 140 extending inwardly therefrom. The axle tube 105 is inserted between the rear hinge plates 139, 140 and the assembly including the lateral plate 113, the rear hinge plates 139, 140, and the axle tube 105 is secured together such as and without being limitative by welding. In the embodiment shown, the axle tube 105 is a tubing having a square cross-section but it is appreciated that it can differ from the embodiment shown. One of the rear wheels 102 is rotatably mounted to axle tube 105 at a lower end thereof through an axle bolt 112 and an axle lock nut 124. The wheel braking assemblies 300 are also mounted to a lower portion of the axle tube 105, above the wheels 102, to control the rotation thereof.

It is appreciated that the configuration of the locking mechanism of the handgrip assembly 265 can vary from the embodiments described above with reference to the accompanying figures.

Still referring to FIG. 3 and, in addition to FIGS. 8A to 8C, each one of the wheel braking assemblies 300 includes a large diameter gear type brake disk 315 mounted coaxially to each one of the rear wheels 102 with mechanical fasteners such as attachment bolts 314. In an alternative embodiment, the rear wheels 102 and the gear disks 315 can be cast as a single component. Each wheel braking assembly 300 further includes a wheel braking housing 313 secured to the rear wheels 102 and to the lateral side portions 195 of the chassis 100. On FIG. 8, for clarity purposes, the wheel braking housing 313 is shown with a cover removed. The wheel braking housing 313 defines an enclosure for several components of the wheel braking assembly 300. The gear disk 315 and the rear wheel 102 are coaxially mounted to an axle mounting point 316 of the housing 313 with a pivot bolt. In the shown embodiment, the gear disk 315 is provided with a plurality of round-ended teeth 304 spaced-apart from one another by a plurality of rounded recesses. The wheel braking housing 313 is opened on one side allowing the wheel stopper 345 and, more particularly, its tooth engaging brake lever 301 to engage with or disengage from the gear disk 315, i.e. to be configured either in the braking configuration or the displacement configuration. The tooth engaging brake lever 301 is pivotally mounted to the wheel braking housing 313 at a pivot mounting point 302. In the embodiment shown, the brake lever is substantially L-shaped with gear engaging teeth 305, which are embodied by two round-ended teeth 305 at an end thereof, the gear engaging teeth being engageable with the complementary recesses of the gear disk 315 in the braking configuration to prevent rotation of the wheel 102. The brake lever 301 is operatively connected to the wheel braking assembly controller 600 through a brake control cable 309 at attachment point 303. The brake control cable 309 is contained inside a brake sheath 310, which can be, for instance, a Teflon® lined sheath. Therefore, by displacement (pulling or released) of the brake cable 309, the tooth engaging brake lever 301 pivots about the pivot mounting point 302 between the braking configuration (i.e. the gear engaging teeth 305 are engaged into the recesses of the gear disk 315) or the displacement configuration (i.e. the gear engaging teeth 305 are disengaged from the recesses of the gear disk 315).

The wheel braking assembly 300 is also provided with a biasing assembly which, in the embodiment shown, includes extension spring 306, mechanically connected to an end 307 of the tooth engaging brake lever 301, opposed to the end including gear engaging teeth 305. The extension spring 306 has a first end 308 secured to the wheel braking housing 313 and a second end secured to the tooth engaging brake lever 301. The extension spring 306 biases the tooth engaging brake lever 301, i.e. the wheel braking assembly 300, into the braking configuration wherein the gear engaging teeth 305 are engaged into the recesses of the gear disk 315. Therefore, when tension in the cable 309 is released, the spring 306 biases the tooth engaging brake lever 301 towards the gear disk 315 into the disengaged/braking configuration. On the contrary, when the brake control cable 309 is tensioned, i.e. pulled up, the tooth engaging brake lever 301 pivots about pivot mounting point 302 into the displacement configuration wherein the gear engaging teeth 305 are disengaged from the recesses of the gear disk 315, thereby allowing rotation of the wheel 102.

In an embodiment, the spring is selected in order to provide sufficient biasing force so that the wheel stopper 345 stays engaged with the gear disk 315 and in accordance with a predetermined force to disengage the wheel braking assembly 300 from the wheels 102.

Referring now to FIG. 25, there is shown a second embodiment for the wheel braking assembly 300. As the above-described embodiment, the wheel braking assembly of FIG. 25 includes a gear disk 315 having a plurality of triangular-shaped teeth 350 configured to engage with a wheel stopper 345 including a tooth engaging brake lever pivotally mounted. Except for the shape of the gear engaging teeth, the tooth engaging brake lever is similar to the tooth engaging brake lever 301 and will not be described in further details.

The use of triangular shaped teeth 350 may provide an improved braking in comparison to a round teeth. From experimentation, it has been observed that when the braking gear disk 315 rotates, the round teeth of the wheel stopper 345 may have a tendency to hover above the braking gear disk 315 and/or bump/bounce against the teeth of the braking gear disk 315, which may result in a delay between a braking command and the engagement of the wheel stopper 345 with the gear disk 315 to prevent a rotation of the wheels 102.

In the embodiment shown in FIG. 25, the braking gear disk 315 has a plurality of triangular-shaped teeth 350 and rounded recesses 351 defined between adjacent ones of the triangular-shaped teeth 350. The triangular shape of the teeth is defined by two sides with an angle varying preferably between 45° and 60° inbetween. The tooth engaging brake lever of the wheel stopper 345 has at least one round-ended tooth 343. In the embodiment shown, the tooth engaging brake lever of the wheel stopper 345 has two round-ended teeth 343 with a pointed tooth receiving recess defined inbetween. Each one of the round-ended teeth 343 has an indentation 346 on each side to retain the tooth 343 into one of the gear disk recesses 351. The round-ended teeth 343 of the tooth engaging brake lever are therefore substantially complementary in shape with the recesses 351 defined between adjacent ones of the triangular-shaped teeth 350 of the gear disk 315. These teeth and teeth receiving profiles may contribute to reduce the hovering effect described above and also lower the loads on the spring attachment points.

It can be appreciated that the gear disk 315 could be provided with a plurality of teeth spaced-apart by a plurality of recesses of any suitable shape while the wheel stopper 345 would have any suitable number of teeth and, optionally, recesses complementary in shape to the ones of the gear disk 315.

It is appreciated that the configuration of the wheel braking assemblies 300 can vary from the embodiments described above with reference to the accompanying figures.

Still referring to FIG. 3 and, in addition to FIGS. 4A to 4C, the pivoting assembly of the chassis 100 will be described. Only one of the pivoting assemblies will be described since the pivoting assemblies of both lateral sides are similar. As mentioned above, the lateral side portions 195 of the chassis 100 are pivotally mounted to the front portion 190 at vertically-extending chassis hinges 106 using pivot bolt 121. Thus, the lateral side portions 195 can pivot about axis 106 between the compacted configuration (FIG. 5A) and the operative configuration (FIG. 3), as shown by arrows 922 (FIG. 4A). More particularly, the hinge plates 139, 140 of the lateral side portions 195 are inserted between the upper and lower reinforcement angle plates 114, 111 of the front portion 190 in a manner such that they can pivot about pivot axis 106.

The rollator 50 comprises a chassis pivoting lock assembly including, amongst other, two sets of square notches 122 defined in the hinge plates 139, 140 of the lateral side portions 195, a hinge lock plate 108 and a hinge lock guiding plate 110. The hinge lock plate 108 and the hinge lock guiding plate 110 are mounted to the front portion 190.

In the embodiment shown, the hinge lock plate 108 is substantially T-shaped. The hinge lock plate 108 is slidably mounted to and between the upper and lower reinforcement angle plates 114, 111 of the front portion 190. More particularly, upper and lower sections 109 of the hinge lock plate 108 are inserted in guiding slots 144 defined in the upper and lower reinforcement angle plates 114, 111 of the front portion 190, with both guiding slots 144 being in register. Thus, the hinge lock plate 108 can translate forwardly and rearwardly into the guiding slots 144. The hinge lock plate 108 also has a forward extension 145 that defines a guiding tongue 145.

Between the upper and lower reinforcement angle plates 114, 111, the front portion 190 of the chassis includes the hinge lock guiding plate 110. The hinge lock guiding plate 110 is mounted to the upper and lower reinforcement angle plates 114, 111 and has two tabs 107. The guiding tongue 145 of the hinge lock plate 108 is inserted between the two tabs 107 of the hinge lock guiding plate 110.

The chassis pivoting lock assembly also includes a biasing means embodied by a compression spring (not shown) located between the tabs 107 and the hinge lock guiding plate 110. The compression spring biases the hinge lock plate 108 in the lock configuration, i.e. rearwardly, wherein it is selectively engageable with one of the two sets of square notches 122 defined in the hinge plates 139, 140 of the lateral side portions 195, in one of the compacted configuration and the operative configuration of the chassis 100.

A rear end of the hinge lock plate 108 is selectively inserted in the first set or the second set of the square notches 122 of the hinge plates 139, 140 of the lateral side portions 195 in a respective one of the compacted configuration and the operative configuration of the chassis 100, thereby locking the chassis 100 in the desired configuration.

To pivot the chassis 100 between the compacted configuration and the operative configuration (or vice-versa), the hinge lock plate 108 is translated forwardly, in direction of arrow 923 (FIG. 4C), by the user to disengage the rear end of the hinge lock plate 108 from one of the two sets of square notches 122. Once disengaged, the lateral side portions 195 of the chassis 100 can be pivoted about pivot axis 106 with respect to the front portion 190. If pressure on the hinge lock plate 108 is released, the rear end of the hinge lock plate 108 automatically reengages one of the two sets of square notches 122 when the chassis 100 is configured in one of the compacted configuration and the operative configuration and lock the chassis 100 in this configuration.

It is appreciated that the pivoting assemblies including the chassis pivoting lock assemblies can vary from the embodiment described above in reference to the accompanying figures.

Still referring to FIG. 3 and, in addition to FIGS. 6A and 6B, in an embodiment, the rollator 50 includes one or more accessory receiving tubes 103. In the embodiment shown, the rollator 50 includes a pair of accessory receiving tubes 103 on each lateral side portion 195. The accessory receiving tubes 103 are vertically mounted to the axle tube 105. As mentioned above, the accessories receiving tubes 103 are configured to receive and support selectively multiples accessories that may be useful to the rollator user. Amongst others, a pair of secondary handgrips assemblies 500 can be received in one of the pair of accessories receiving tubes 103, such as the forward ones on each lateral side of the rollator 50.

Each one of the secondary handgrip assemblies 500 includes a secondary handgrip frame 502 inserted in the forward one of the accessory receiving tubes 103. In the embodiment shown, the accessory receiving tubes 103 are modified to receive the secondary handgrip frames 502 and will be referred to hereinbelow as handgrip receiving tubes 505. As standard accessory receiving tubes 103, the handgrip receiving tubes 505 are embodied by a vertical square tubing, and are mounted, such as by welding, to a rear vertical surface of the axle tube 105. A respective one of the secondary handgrip frames 502 is inserted and partially contained within a respective one of the handgrip receiving tubes 505. In an embodiment, the secondary handgrip frame 502 is translatable, along arrow 926, in the handgrip receiving tube 505 in order to modify a height of the secondary handgrip assemblies 500 in accordance with a height of the rollator user.

To be adjustable in height, each of the handgrip receiving tubes 505 has, on one side thereof, a vertically extending opening 507 to receive a height adjustment indicator 504, which, in turn, is mounted to the handgrip frame 502. The height adjustment indicator 504 indicates a relative position of the secondary handgrips 509 with respect to the opening 507. It can be appreciated that a ruler decal can be provided next to each of the openings 507 so that the user or a medical professional can precisely adjust the height of the secondary handgrips 509 with respect to the ground.

In a lower portion thereof, each handgrip receiving tube 505 includes a bottom plate 508 having a circular opening defined therein, The bottom plate 508 is secured to the handgrip receiving tube 505 with a threaded screw 503 and a locking nut (not shown) positioned inside of the vertical portion of the handgrip receiving tube 505. A lower end of each handgrip frame 502 is provided with a threaded end that is engaged by a threaded screw 503 to adjust the height of the handgrip frame 502 with respect to the handgrip receiving tube 505. Fine tuning of the height of the handgrips 509 can be done by screwing or unscrewing the threaded screw 503 as indicated by arrow 925. In a non-limitative embodiment, the threaded screw 503 is a hexagonal drive screw. At an upper end, each handgrip frame 502 has a horizontal handgrip section 506 that is substantially cylindrical in shape and extends towards the front portion 190 of the chassis 100. Each of the horizontal handgrip sections 506 is provided with a rubber handgrip 509 for comfort of the user. The secondary handgrips 509 can be used to provide support to a user when standing up from a seated position, to sit on the rest seat or on a toilet seat as shown on FIG. 21 and as described in more details below.

As shown in FIGS. 3, 6A and 6B, another pair of accessories receiving tubes 103 can be mounted to the handgrip receiving tube 505 or to any other suitable sections of the chassis 100.

Referring now to FIGS. 7A and 7B, the primary handgrip assemblies 200 will be described in further details. Since both handgrip assemblies 200 are similar, only one will be described and it is appreciated that the description applies to both. The primary handgrip assembly is provided with a height adjustment and a fall detection system. In FIGS. 7A and 7B, a cover of the handgrip control box 225 is removed to understand the internal components. As mentioned above, the handgrip assembly includes a handgrip frame 202, which in the embodiment shown is a substantially L-shaped tube having a square cross-section with a tubular portion 205 at an end thereof to define a pivot axis. More particularly, the handgrip frame 202 is pivotally mounted to the front portion 190 of the chassis 100 through the tubular portion 205 through a pivot bolt (not shown) and a low friction round plastic bushing (not shown). The tubular portion 205 is mounted coaxially to one of the pivot point of attachment 116 or 135 of the front portion 190.

The L-shaped square section of the handgrip frame 202 has, at its opposed end, a threaded female insert centered and weld to mount the handgrip control box 225 thereto via the use of a height adjustment screw 218. The cross dimensions of the L-shaped bended tube 202 are chosen or machined so that the corresponding vertical sliding square tube 217 of the control box 225 can slide over it with low friction. The handgrip control box 225 is defined by the assembly of a back plate 230, side plates 231, a middle plate 227, a top plate 226 and a bottom plate 225. A weight rod transmission housing 234, embodied by a square tube, is mounted inside the box 225 inbetween the top plate 226 and the middle plate 227. The vertical sliding tube 217 is secured to, such as by welding, to the middle and the bottom plates 227, 228.

The vertical sliding tube 217 is provided with a vertical opening 222 in which a button shaped height adjustment indicator 221 can travel to give the user or medical professional an indication of the height adjustment. A corresponding opening is provided on the control box cover (not shown) and can have a numbered ruler mounted parallel to the opening giving a numbered reading of the height.

A height adjustment screw 218 is inserted into a round opening of the top control box plate 226 with a low friction round plastic washer 219 placed inbetween the head of the height adjustment screw 218 and the corresponding opening of the top control box plate 226. A second low friction washer 219 is placed underneath the top plate 226 opening and the screw 218 is secured with a lock nut 220 to form a sandwich type assembly. The lock nut 220 is screwed in place so that the residual gap is minimal while the height adjustment screw 218 can still rotate freely as indicated by arrow 928.

A low friction square opening plastic bushing 216 is inserted into the weight rod transmission container 234 and secured in place. A vertically-extending rod weight transmitter 206 is mounted to and inserted inside the handgrip control box 225 and engaged with the low friction square opening bushing 216. The dimension tolerances are selected in a manner such that the rod 206 can translate upwardly and downwardly, as shown by arrow 927 inside the bushing 216 with low friction. The rod weight transmitter 206, is provided with a centered threaded hole, at a lower end thereof, which is engaged by a weight detection adjustment screw 215. A compression spring 212 is mounted to the rod weight transmitter 206, between the lower control box plate 228 and the lower end of the square rod weight transmitter 206. The compression spring 212 is selected to control the pressure required to activate the wheel braking system 650, especially in a case of fall detection. The tension in the compression spring 212 can be adjusted by rotating the adjustment screw 215 about arrow 930. The compression spring biases the handgrip 204 in the raised configuration.

The rod weight transmitter 206 includes a pivot hole 207 at a top end thereof extending along an axis perpendicular to a longitudinal axis of the rod weight transmitter 206. The handgrip 204 is pivotally mounted to the rod weight transmitter 206 through this pivot hole 207. The handgrip 204 is provided a lower portion 233, close to the pivot hole 207, that limits a downward pivoting movement of the handgrip 204 by interfering with the top section of the rod weight transmitter 206. Therefore, the handgrip 204 cannot pivot below a horizontally extending position, as shown in FIG. 12. More particularly, the handgrip can pivot of an angle α, between position 901 and position 900.

Returning now to FIGS. 7A and 7B, the handgrip 204 has a second opening 208 to which a handgrip control cable 209 is connected. In a non-limitative embodiment, the control cable 209 is a stainless steel multi strand cable. This handgrip control cable 209 is contained inside a handgrip sheath 211, such as and without being limitative, a low friction Teflon® lined sheath, that is mechanically connected to the wheel braking assembly controller 600 mounted to the front portion 190 of the rollator 100. The sheath 211 extends through the handgrip control box 225 by a hole defined in the bottom control box plate 228, and extends through the middle control box plate 227 via a hole coaxially positioned. The end of the sheath 211 is compressed against a sheath receptor fitting 210. The sheath receptor fitting 210 is cylindrical in shape with threads on the outside of the barrel. It has a hole in the inside that allows the control cable 209 to travel in and an enlarge bottom end with an inside diameter hole that allows the extremity of the sheath 211 to enter the hole. This larger bottom end has a knurled outside surface that allows the fitting to be turned by hand. This sheath receptor fittings 210 allows for tension adjustment.

The height of the handgrips 204 can be finely adjusted according to the user height. The height adjustment screw 218 can be turned either clockwise or counterclockwise about arrow 928 to translate a position of the handgrip control box 225 vertically along arrow 929.

Turning now to FIG. 12, it is shown that the handgrip 204 is biased into position 900. It can be depressed manually into position 902 wherein the handgrip 204 extends substantially horizontally. When pivoted downwardly into position 902, the control cable 209 is pulled along arrow 931 (FIG. 7B), thereby transmitting a command to the wheel braking assembly controller 600.

If the user applies an abnormal pressure on the handgrip 204, i.e. a pressure equal to or greater than a handgrip pressure threshold, that overcomes the resistance of the compression spring 212, then the vertical square rod weight transmitter 206 will be pushed down along arrow 927 in a way that it neutralizes the displacement of the control cable 209. Once again, a command is transmitted via the control cable 209 to the wheel braking assembly controller 600. The pressure required to overcome the resistance of the compression spring 212, i.e. the handgrip pressure threshold, can be adjusted by increasing the preload of the compression spring 212 by turning the weight detection adjustment screw 215 either clockwise or counterclockwise along arrow 930.

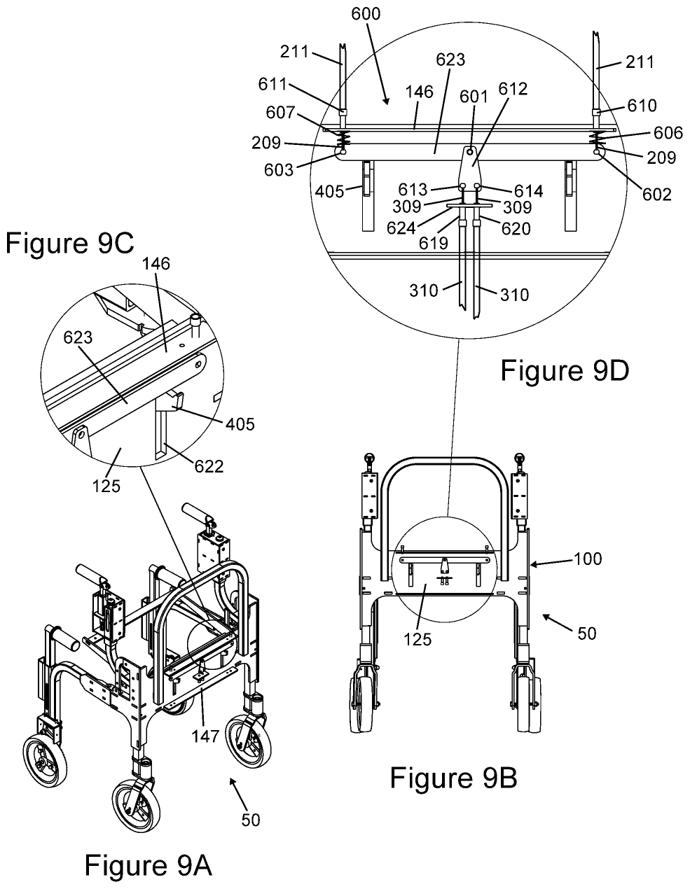

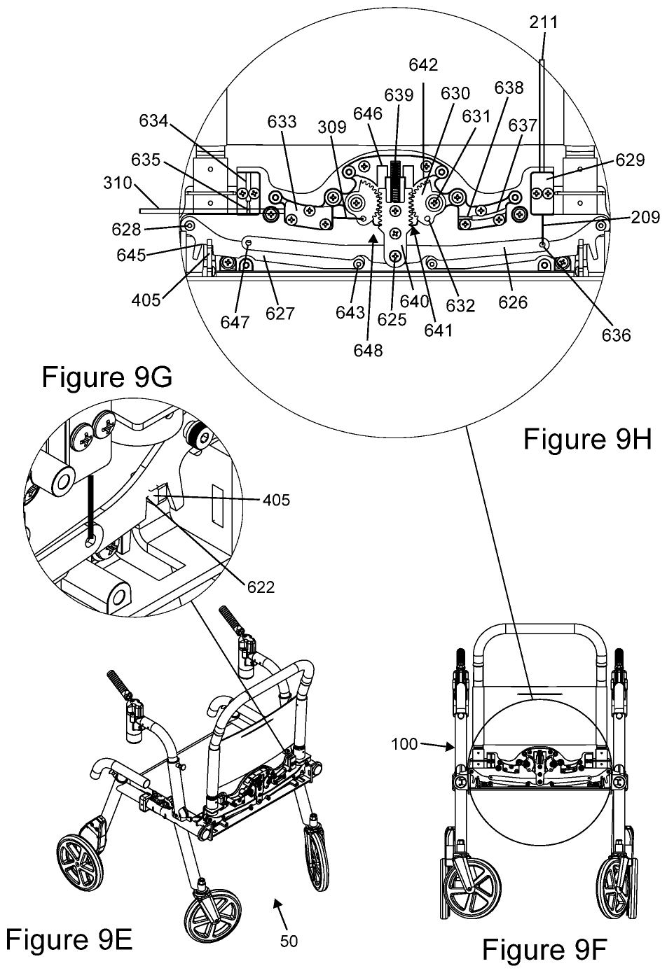

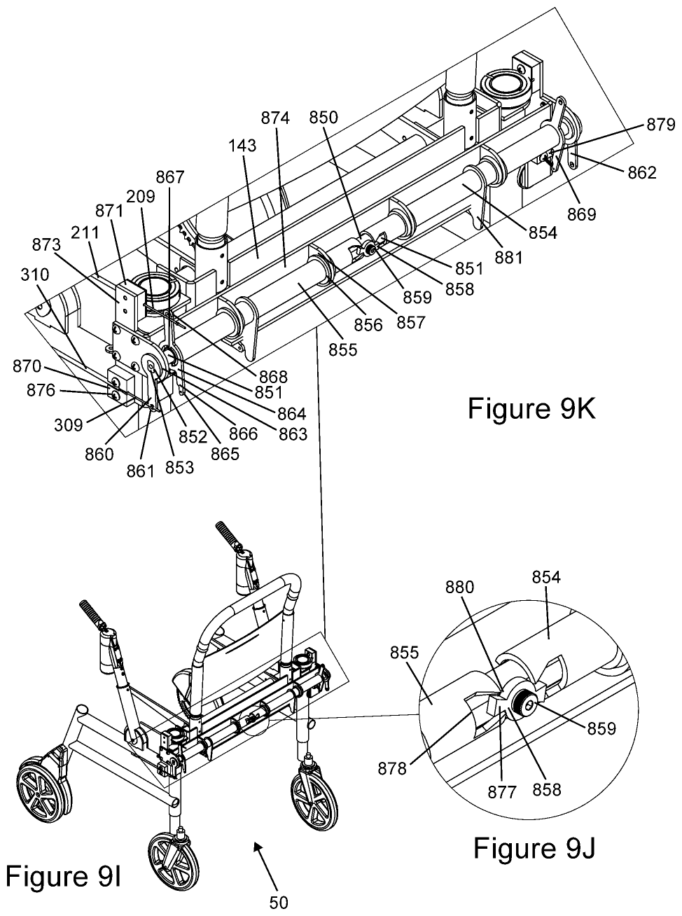

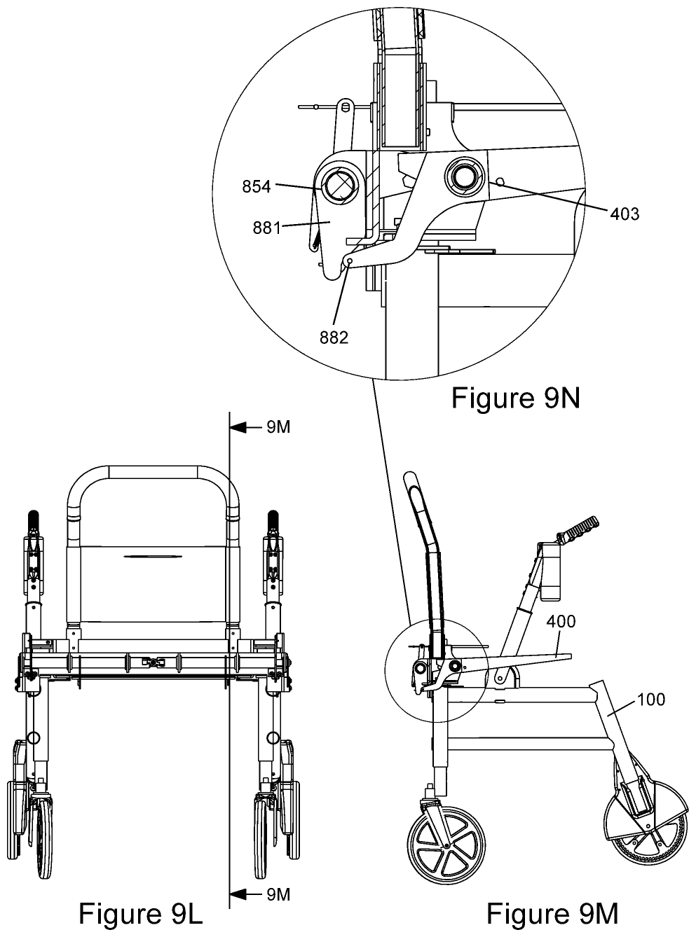

Referring now simultaneously to FIG. 3 and FIGS. 9A to 9D, there is shown a first embodiment of the wheel braking assembly controller 600, or hazard control mechanism, which is mounted to an outside (front) face of the front plate 125. The wheel braking assembly controller 600 is designed to detect different kind of hazard that may lead to falls, which will be described in further details below. In the embodiment shown, the front plate 125 is provided with four vertically-extending openings. Two first openings 142 (FIG. 3) are symmetrically positioned with the rest seat transverse center line. The lateral structural ends 404 of the rest seat 400 are at least partially inserted in a respective one of the first openings 142 and are designed to limit the downward pivoting movement of the seat 410, i.e. to prevent the rest seat 410 to extend substantially below a horizontal configuration.

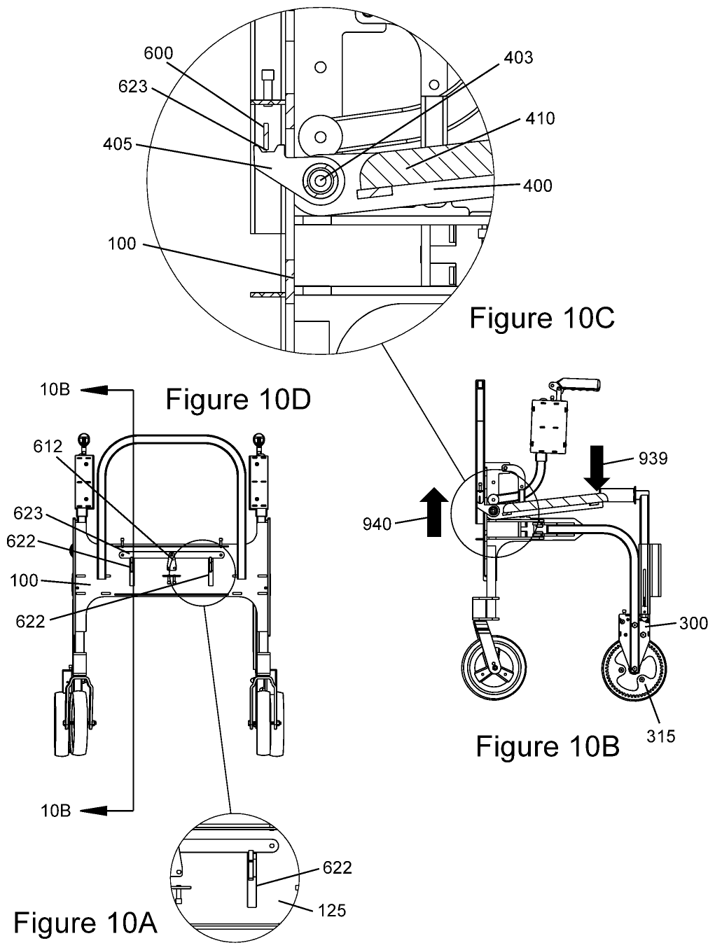

A second pair of vertical openings 622 is located inside of the first set of openings 142. This second pair of vertical openings 622 allows two brake lifters 405 to pivot within. More particularly, the two brake lifters 405 are used to control a safety braking system when the user is seated (safe condition) and is used as part of the anti-roll back system in the wheelchair or transport chair mode. The safety braking system will be described in more details below in reference to FIGS. 10A to 10C.

Still referring to FIGS. 9A to 9D, an embodiment of the wheel braking assembly controller 600 is shown, wherein a cover has been removed. Top and bottom plates 146, 147 are mounted to the front plate 125 and extend substantially parallel to each other and substantially horizontally. They are spaced-apart from one another to define an internal spacing wherein several components of the wheel braking assembly controller 600 are mounted. The top and bottom plates 146, 147 are substantially flat bars mounted normal to the front plate 125. The top plate 146 has threaded apertures defined therein at opposed ends thereof. Primary adjustable sheath receptor fittings 610, 611 (a left one and a right one) are screwed in a respective one of the threaded apertures defined in the top plate 146.

In the internal spacing defined between the top and bottom plates 146, 147, a sheath receptor fitting attachment plate 624 is mounted to the front plate 125 and extends perpendicularly thereto and substantially centrally. In the embodiment shown, the attachment plate 624 is a flat bar with two threaded apertures symmetrically positioned apart of the center line and perpendicular to its surface. Brake adjustable sheath receptor fittings 619, 620 (a left one and a right one) are screwed in a respective one of the threaded apertures defined in the attachment plate 624.

The wheel braking assembly controller 600 further includes a central floating balancer plate 623, which is substantially rectangular in shape. The balancer plate 623 has a pivot mounting round aperture 601, defined substantially centrally, and a right and left cable attachment points 602, 603 located respectively at its left and right ends. The attachment points 602, 603 are showing a horizontally oriented oblong shape with the flat surfaces being horizontal and parallel to the longest rib of the balancer plate 623. These oblong holes allow for right and left handgrip control cables 209 to be fastened at the corresponding connecting points 602, 603 of the balancer plate 623 using cable attachment bolts.

The wheel braking assembly controller 600 also includes compression springs 606, 607 mounted to a respective one of the handgrip control cables 209. The balancer plate 623 also has perpendicularly extending top tabs at each end, in which an aperture would permit the cables 209 to go through to connect with the connecting points 602, 603. This allows a better and more stable contact of the compression springs 606, 607 to the balancer plate 623.

The wheel braking assembly controller 600 also includes a safe condition transmission plate 612 to which the balancer plate 623 is pivotally connected at its centered pivot hole 601 through a pivot bolt (not shown). In the embodiment shown, the safe condition transmission plate 612 has a truncated triangular shape with the pivot hole 601 defined at its upper end and two horizontally oriented oblong holes 613, 614 (a left one and a right one) at its lower end. Brake control cables 309 (a left one and a right one) are connected to the safe condition transmission plate 612 through a respective one of the two horizontally oriented oblong holes 613, 614 by the mean of cable attachment bolts. The brake control cables 309 extend inside a respectively one of the sheath receptor fittings 619, 620 (a left one and a right one) and inside a respective brake sheath 310, such as and without being limitative a Teflon® lined sheath. Each one of the brake control cables 309 is attached at its other end to its corresponding attachment point 303 of the respective wheel brake assembly 300.

The balancer plate 623 is mechanically connected to the right and left handgrips 204 through the control cables 209 attached at the balancer attachment points 602, 603 with the use of cable attachment bolts. The handgrip control cables 209 extend in adjustable sheath receptor fittings 610, 611 and inside handgrip sheath 211, such as and without being limitative a Teflon® lined sheath, towards the corresponding primary handgrip assembly 200.

The two compression springs 606, 607 (respectively the right and left compression springs) are coaxially positioned around respectively the right and left handgrip control cables 209 between the top plate 146 and the corresponding right or left end of the balancer plate 623 (perpendicular with the horizontally frontward extending right or left tab) and maintain a constant down pressure on the balancer plate 623 at the right and left ends.

Turning now to FIGS. 10A to 10C, other features or components of the wheel braking assembly controller 600 will be described and, more particularly, relative to the anti-roll back functionality associated to the rest seat 410. As mentioned above, a second pair of vertical lifter openings 622 is located inside of the first set of openings 142. This second pair of vertical openings 622 allows two brake lifters 405 to pivot within while the seat 410 is pivoted between the seated configuration and the raised configuration. More particularly, the two brake lifters 405 are mounted to the pivoting tube 403 of the seat assembly 400 and pivot therewith. They are horizontally centered in the vertical lifter openings 622 defined in the front plate 125. Thus, when a user pivots the rest seat assembly 400 towards the seated configuration along arrow 939, the rest brake lifters 405 will abut against the balancer plate 623. However, since the pressure applied on the seat 410 is insufficient to reach the seat pressure threshold, the compression springs 606, 607 will retain the balancer plate 623 position so that the rear wheel brake assemblies 300 remained engaged with their respective rear wheel 102, i.e. in the brake configuration. Therefore, the rollator 100 remains stationary and motionless.

When the user is sitting down and that the pressure applied downwardly (along arrow 939) by its weight on the rest seat assembly 400 is sufficient to overcome the resistance of both compression springs 606, 607 together with the additional resistance of both wheel brake extension springs 306 (i.e. the pressure applied on the seat 410 is equal to or greater than the seat pressure threshold), then the brake lifters 405 will push upwardly (along arrow 940) both ends of the balancer plate 623. The vertical displacement of the balancer plate 623 will raise the safe condition floating transmission plate 612 which, in turn, will pull both the brake control cables 309 to disengage the wheel control assemblies 300. When the wheel control assemblies 300 are disengaged, the rollator 50 is allowed to move freely. In this situation, the wheel braking assembly controller 600 interprets that the user is safely seated and ready to move using his feet to propel himself or the push rims 042 in the wheelchair version (FIG. 26).

When the user attempts to stand up, and that the weight (or pressure) applied on the rest seat 400 is insufficient to overcome the spring resistance (606, 607 and both wheel brake extension springs 306), i.e. the pressure applied on the seat 410 is below the seat pressure threshold, then the springs 606, 607, 306 automatically configure the wheel control assemblies 300 into the brake configuration, which is safer for the user. This situation is interpreted by the wheel braking assembly controller 600 as a user tentative to stand up, which requires that the rollator 50 be configured into the brake configuration in order to give a stable and motionless position to the rollator 50.

FIGS. 9D to 9F shown an alternative embodiment of the wheel braking assembly controller 600. This embodiment is similar in many aspects to the embodiment described above but can be designed more compact and may provide a safer control for the disengagement of the wheel braking assembly 300 while using the seat 410 in the seated configuration, i.e. the horizontal configuration. In comparison with the first embodiment, the wheels braking cables 309 partially enclosed in sheaths 310, are extending substantially horizontally along the front portion 190 which allows to free the space underneath the front plate 125. With this substantially horizontal configuration, the wheel braking cables 309 and sheaths 310 are not bent with a sharp radius that could increase resistance for the travelling of the cables 309.

This second embodiment includes two lateral levers 627 that may provide a better control of the position of the seat 410 at which the wheel braking assemblies should be configured in the displacement configuration.

Referring to FIGS. 9G and 9H, the wheel braking assembly controller 600 is shown with the cover removed. Furthermore, for clarity purposes, only one control cable for each set of control cables, i.e. one wheel braking control cable 309 and one primary handgrip control cable 209 together with their respective sheaths 310, 211, is shown but it is understood that the wheel braking system includes a pair of each control cable and sheath for movement transmission on both sides of the rollator 50.

The wheel braking assembly controller 600 includes a front plate 648, mounted to the front portion 190 of the chassis 100 and onto which most of the components of the wheel braking assembly controller 600 are mounted or engaged. In an embodiment, the front plate 648 is a plastic molded plate. The front plate 648 can include steel reinforcements, such as a steel reinforcement plate, at the attachment points.

The wheel braking assembly controller 600 includes a central floating balancer plate 626 having a generally elongated “banana” shape with a central pivot hole 625 and one cable attachment point at each end 636, 647. The banana shape allows for the balancer plate 626 to pivot up and down in a more compacted way. The attachment points 636, 647 are characterized by an oblong shape with the flat surface being horizontal. Each of the attachment points 636, 647 is linked to a corresponding one of the left and right handgrip cables 209 as per to transmit the primary handgrip movement to the balancer plate 626. The balancer plate 626 is connected at its center pivot hole 625 to a safe condition transmission plate 640. The safe condition transmission plate 640 has an elongated shape with a pivot attachment point at a lower end thereof (which is aligned with the central pivot hole 625 of the balancer plate 626). The safe condition transmission plate 640 has gear racks 641 on both sides. The wheel braking assembly controller 600 also includes brake cable driving gears 630 pivotally mounted to the front plate 648 at pivot attachment point 631, each one being mounted on a respective side of the safe condition transmission plate 640. Each one of the brake cable driving gears 630 has gear teeth 642 that are operatively engaged with the teeth of a respective one of the gear racks 641 of the safe condition transmission plate 640. The wheel braking assembly controller 600 also includes a compression spring 639 operatively connected to an elongated rectangular extension of the safe condition transmission plate 640. The compression spring 639 biases downwardly the safe condition transmission plate 640 in order to maintain it to its lowest position when no external forces/pressure is applied on it. The front plate 648 includes a cavity 646 guiding a vertical displacement of the safe condition transmission plate 640. Each one of the brakes cable driving gears 630 has a central pivot 631 around which the gear 630 pivots. Finally, each of the brake cable driving gears 630 has an oblong eccentric attachment point 632 to which the wheel braking cable 309 is attached.

The wheel braking cables 209, 309 are partially enclosed in a respective sheath 211, 310, such as and without being limitative a Teflon® lined sheath. The sheath 211 is secured into a plastic cavity 637 that is molded in the front plate 648. The plastic cavity 637 has a substantially U-shape, slightly smaller in diameter than the sheath 310 so that the sheath 310 is slightly squeezed therein. The cavity 637 has a fully opened end on a first side and a reduced diameter passage 638 on a second side. This reduced diameter passage 638 allows for the control cable 309 to extend therethrough while retaining the sheath 310 from sliding frontward. The cavity 637 is covered with a cover 633, such as a metallic cover, that is secured to the front plate 648, such as by screwing, in order that the sheath 647 cannot be detached. In FIG. 9H, the cover 633 is removed on a right side to expose the cavity 637 and the reduced diameter passage 638, while the cover 633 is secured to the front plate 648 on the left side.

The primary handgrip cable 209 and its sheath 211 are attached similarly to the wheel braking cable 309 and its sheath 310 to the plastic front plate 648 with the sheath 211 lightly squeezed into a U-shaped molded channel 634 with a reduced diameter lower portion 635 that limits the movement of the sheath 211. The sheath and molded cavity are then covered with a metallic plate 629 and secured in place, such as by screwing. Each of the primary handgrip cables 209 has a compression spring (not shown) positioned coaxially around and adjacent to the balancer plate 626, the compression spring maintains the balancer plate 626 in a lowered configuration.

The anti-roll back system of the wheel braking assembly controller 600 still includes brake lifters 405 of the seat assembly 400 engaged in a respective aperture 422 defined in the front plate 648. The brake lifters 405 and their respective apertures 422 are located on each lateral side of the front plate 648. The anti-roll back system further includes two levers 627, including an elongated plate, also located on each side of the front plate 648 and pivotally mounted thereto at pivot 628. The elongated plates of the levers 627 are also provided with a lifter 643 located at an end close to a center of the front portion 190 and a U-shaped cut 645 defined in a lower edge of each lever 627. The U-shaped cut 645 are engageable by the lifter 645 of the seat assembly 400 in a manner such that the levers 647 can be pushed upwardly downward pivoting of the seat 410. Thus, when the seat 410 is lowered toward its seated configuration, i.e. the horizontal configuration, the lifters 644 engage with the levers 627, but the resistance of the compression springs prevent the wheel braking assembly 300 from disengaging from the wheels 102. In this configuration, the seat 410 extends slightly upwards with respect to its lowest configuration. In an embodiment, the seat 410 defines a 2° angle with respect to a ground surface. In this configuration, the seat 410 is ready for a user to sit thereon with the wheel braking assemblies 300 engaged with the wheels 102. When a user is sitting on the seat 410 in such a way that the user's weight transferred to the seat 410 exceeds the compression springs resistance, i.e. the pressure on the seat 410 is above the seat pressure threshold, the brake lifters 644 push the levers 627 upwards. The levers 627 then engage with the balancer plate 626 and push it upwards. As a result, the safe condition transmission plate 640 is pushed upwards and the two brake cable driving gears 630 rotate accordingly, thereby pulling the wheel braking cables 309 and disengaging the wheel braking assemblies 300 from the wheels 102.

Referring now to FIGS. 9I to 9K, there is shown a third embodiment of the wheel braking assembly controller 600. This alternative embodiment is different from the embodiments described above in that it uses a rotational movement of a control mechanism and is provided with less components. As the embodiment described in reference to FIGS. 9E to 9H, the embodiment of FIGS. 9I to 9K is provided with a substantially horizontal discharge orientation of the control cables and their respective sheaths. This embodiment may reduce a length and quantity of bent portions of the control cables directed towards both the wheel braking assemblies 300 or the primary handgrip assemblies 400.

The wheel braking assembly controller 600 includes a base plate 874 mounted to the front portion 190 of the chassis 100, side plates 863 extending forwardly of the base plate 874 on each lateral end thereof, and a main shaft 850 rotatably mounted into a pair of low friction plastic bushings located within the side plates 863. The main shaft 850 has a central cylindrical portion and opposed side portions. The central cylindrical portion 851 is substantially flat with a bow-shaped joint 858 (or bow tie or butterfly-shaped joint) protruding therefrom. The bow-shaped joint 858 is pivotally mounted to the central cylindrical portion 851 at pivot attachment point 859 using a shoulder bolt acting as a pivot. The main shaft 850 has a first diameter in the central portion 851 with a diameter that reduces in the side portions, i.e. towards both lateral ends. In an embodiment, the ends 852 of the main shaft 850 are machined with an oblong, square or rectangular shape.

The wheel braking assembly controller 600 also includes a pair of brakes driving plates 860, 862 (one for each lateral end 852). Each one of the brakes driving plates 860, 862 has a circular portion provided with an oblong or irregular aperture in its center that corresponds to the shape of the end 852 of the main shaft 850. Therefore, the ends 852 are inserted in a respective one of the apertures defined in the center of the brakes driving plates 860, 862. In an embodiment, at the lateral ends 852, the main shaft 850 is provided with a central tapped hole 853 that allows to put a washer and screw (not shown) to secure brakes driving plates 860 thereto. Therefore, the rotational movement of the main shaft 850 about its longitudinal axis is transferred to the brakes driving plates 860, 862. The brakes driving plates 860, 862 are also provided with an eccentric extension having an oblong aperture at an end thereof that allows the brake control cables 309 to be attached to the brakes driving plates 860, 862 using a cable attachment device (not shown). As detailed above, the brake control cables 613 drive the wheel braking assemblies. As in the above-described embodiments, the wheel braking cables 309 travel through a sheath 310, such as a Teflon® lined sheath, that has an end secured into an immobilisation plastic bloc 870 mounted to the base plate 874. The sheath 310 is compressed into the immobilisation plastic bloc 870 with a metal plate (not shown) and screws 876.

The wheel braking assembly controller 600 also includes two outer tubes 854, 855 (or handgrip motion transfer tubes), having a section of the main shaft 850 inserted therein and being respectively located one both lateral sides of the bow-shaped joint 858. These outer tubes 854, 855 have an internal diameter that allows the main shaft 850 to rotate therein without interference. The outer tubes 854, 855 are supported with four tube supports 857 which have a substantially “D” shape with a round opening defined therein in which a low friction plastic bushing 856 is inserted and surrounds the outer tubes 854, 855 to allow rotation of the outer tubes 854, 855 with low friction. Hereinbelow, only one of the outer tubes 854, 855 will be described since both are similar. The outer tubes 854, 855 have, at a central end thereof, an indentation 878 with two spaced-apart contact points 880 at an end thereof. A handgrip driving lever 864 is mounted to the lateral side end of each of the outer tubes 854, 855. The handgrip driving lever 864 has a central section 867 with round opening defined therein to be engaged by one of the outer tubes 854, 855. Each one of the outer tubes 854, 855 has handgrip driving lever 864 secured to its lateral end. Therefore, the outer tubes 854, 855 and their handgrip driving lever 864 rotate simultaneously.

Each one of the handgrip driving levers 864 is provided with two opposed eccentric extensions. A first one of the eccentric extensions, the upper one, has a cable attachment point 868 at an end thereof to which the handgrip motion control cable 209 can be attached. A second one of the eccentric extensions, the lower one, is also provided with a hole 865 at an end thereof. An extension return spring 866 can be attached to the second eccentric extension through the hole 865. The extension return spring 866 biases the respective one of the outer tubes 854, 855 into its rest configuration, which corresponds to the braking configuration of the wheel assemblies 300. As in the above described embodiments, the handgrip motion control cable 209 travel through a sheath 211, such as a Teflon® lined sheath, that has an end secured into an immobilisation plastic bloc 871 mounted to the plate 873. The sheath 211 is secured to the immobilisation plastic bloc 871 with a metal plate (not shown) and screws.

The horizontal positioning of outer tubes 854, 855 is ensured with a plastic bloc 879, mounted to the base plate 874, that maintains the handgrip driving levers 864, 869 aligned.

As shown, the bow-shaped joint 858 has two triangular lateral extensions 877 that are inserted into a respective one of the indentations 878 defined in the central ends of the outer tubes 854, 855. Upon rotation of the outer tubes 854, 855 along their longitudinal axes, the contact points 880 contact the bow-shaped joint 858 and engages the main shaft 850 and its bow-shaped joint 858 into rotation about its longitudinal axis.

When only one of the primary handgrips 204 is configured into the handgrip displacement configuration (i.e. the lowered configuration), then only one of the outer tubes 854, 855 is engaged into rotation. One of the contact points 880 of the outer tubes 854, 855 engaged into rotation, pushes only one side of the bow-shaped joint 858 which rotates onto itself about pivot attachment point 859 without transferring the rotational motion to the main shaft 850 and, therefore, without pulling the brake control cables 309. Therefore, the wheel brake assemblies 300 of the rollator 50 remain in the braking configuration.

When both primary handgrips 204 are configured into the handgrip displacement configuration (i.e. the lowered configuration) and the pressure applied thereon is below the handgrip pressure threshold, then both outer tubes 854, 855 are engaged into rotation. Both sides of the bow-shaped joint 858 are contacted by the contact points of the outer tubes 854, 855 and pushed. Being pushed on both sides, the bow-shaped joint 858 cannot rotate onto itself about the pivot attachment point 859, the rotational motion is thus transferred to the main shaft 850 which rotates about its longitudinal axis. Upon rotation of the main shaft, the brake control cables 309 are pulled on both lateral sides. Therefore, the wheel brake assemblies 300 are disengaged, i.e. configured into the displacement configuration, and the rollator 50 is free to be displaced.

In this embodiment, as shown in FIGS. 9L to 9N, the seat 410 is linked to the outer tubes 854, 855 in a way that, when the seat 410 is in its horizontal configuration and the seat pressure threshold is met, both outer tubes 854, 855 rotate simultaneously and thereby to disengage the wheel braking assemblies 300. More particularly, the seat assembly 400 is provided with two brake lifters 405 secured to the pivoting tube 403 of the seat assembly 400 and rotating simultaneously therewith. The brake lifters 405 are provided with an aperture 882 at a front end thereof. Each one of the outer tubes 854, 855 has a seat control rotation plate 881 secured thereto in a manner such that rotation of one of the seat control rotation plates 881 engages the respective one of the outer tubes 854, 855 into rotation. Thus, when the seat 410 in the seated configuration with a pressure equal to or greater than the seat pressure threshold, the brake lifters 405 pushes the seat control rotation plates 881, which in turn engages outer tubes 854, 855 into rotation. As detailed above, simultaneous rotation of the outer tubes 854, 855 configures the wheel brake assemblies 300 into the displacement configuration.

In another alternative embodiment, the wheel braking assembly controller 600 can be an electric/electronic version including a power supply mounted to the chassis 100, handgrip sensors, such as limit switches, operatively connected to the handgrips 204 and configured to sense/detect a configuration of each one of the handgrips 204 (either the handgrip walking/displacement configuration or the braking configuration), a seat sensor, such as a limit switch, configured to sense/detect a configuration of the seat (either the seated configuration with no or limited pressure applied thereon, the seated configuration with pressure above a seat pressure threshold applied thereon—or a lower seated configuration), a logic controller operatively connected to the handgrip sensors and the seat sensor, and a plurality of electric connectors (i.e. electric control cables) connecting the handgrip sensors and the seat sensor to the logic controller and to the wheel braking assemblies 300. In an embodiment, the power supply is operatively connected to at least one of the handgrip sensors, seat sensor, the logic controller and the wheel braking assemblies and provides electrical power thereto.

It is appreciated that the configuration of the wheel braking assembly can vary from the embodiments described above in reference to the accompanying figures and combinations of the different embodiments can be foreseen.

It is appreciated that the control cables can be either mechanical control cables or electric control cables depending on the type of wheel braking assembly controller 600.

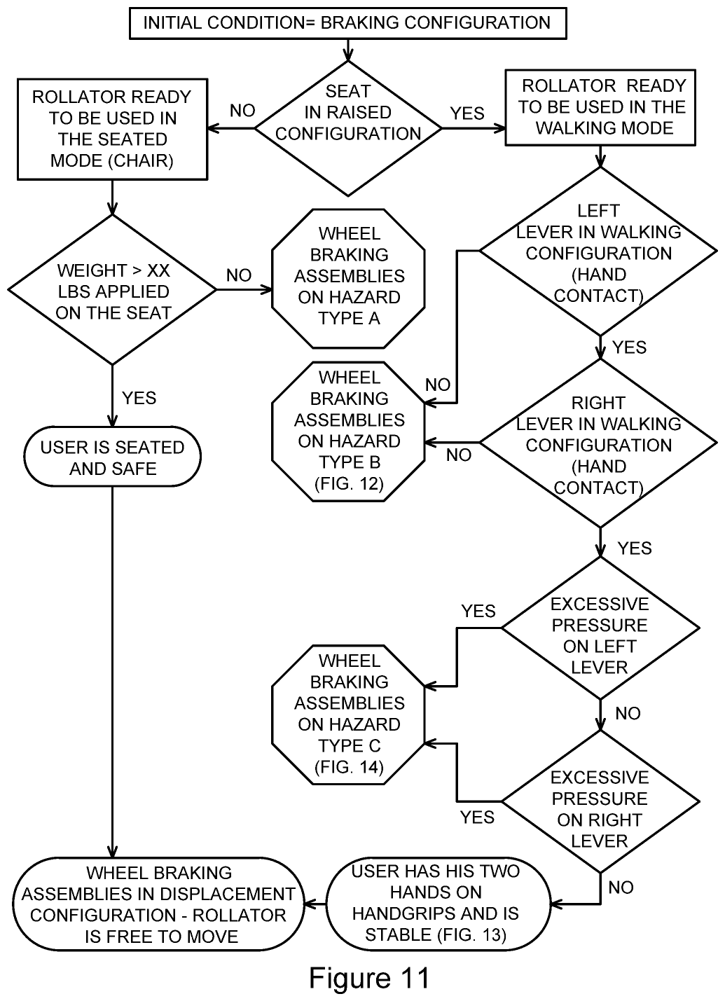

Referring now to FIG. 11, there is shown a flowchart of the operating modes of the wheel braking system 650 including the wheel braking assembly controller 600, the handgrip control cables 209 and their sheaths 211, the brake control cables 309 and their sheaths 310, the anti-roll back system operatively connected to the seat assembly 400, and the wheel braking assemblies 300.

In FIG. 11, three types of hazards (A, B and C) in relation with user's hand behavior and seated position are described in relation with the configuration of the wheel braking assemblies 300 of the rollator 50. By default, the wheel braking assemblies 300 of the rollator 50 are always configured in the braking configuration when no external forces or pressure is applied either on the handgrips 204 or the seat 410.

A first condition relates to a configuration of the seat 410 of the seat assembly 400. If the seat is configured in the seated configuration with no or low pressure applied thereon, the wheel braking assembly controller 600 interprets this condition as the intention of the user to sit down. The wheel braking assemblies 300 will remain in the braking configuration unless the user is seated, i.e. the seat 410 is further pivoted downwardly or a pressure equal to or greater than the seat pressure threshold is applied on the seat 410. If the seat 410 is further pivoted downwardly along arrow 939 (FIG. 10) or a pressure equal to or greater than the seat pressure threshold is applied on the seat 410 to exceed a pre-set resistance of the corresponding compression springs 606, 607 and the corresponding extension springs 306 of both right and left wheel braking assemblies 300, then the wheel braking assemblies 300 are configured in the displacement configuration, i.e. disengaged from the wheels 102. In a non-limitative embodiment, the pressure applied on the seat 410 should exceed 15 lbs. Should the weight/pressure applied on the seat 410 be less than the minimum required weight/pressure, the wheel braking assembly controller 600 interprets this condition as a “type A hazard”. The “type A hazard” is described as one of the following situations: the user is either not completely seated or the user is trying to stand up in which case the rollator 50 should be in the braking configuration to assure that the rollator 50 will not roll away. If the weight/pressure applied on the seat 410 exceeds the pre-set value, then the user is considered as being seated and is then in a safe position where fall cannot occur. In this case, the rollator 50 is configured in the displacement configuration, i.e. free to move.

Otherwise, if the seat 410 is configured in the raised (i.e. substantially) vertical configuration, then this condition is interpreted as the rollator 50 is ready to be used in the walking mode. Therefore, the wheel braking assemblies 300 of the rollator 50 will remain in the braking configuration until the safety conditions are met (right side of the flowchart).

Then, the first set of conditions to meet are the configurations of the primary handgrips 204. If only one of the handgrips 204 is configured in the handgrip walking/displacement configuration, then the wheel braking assembly controller 600 interprets this condition as a “type B hazard”, which is further detailed in reference to FIG. 12. In a “Type B hazard”, the rollator user has only one hand in contact with the rollator 50, the second hand could be grabbing something on a table, a counter or in a refrigerator and in all of those cases, the user should have a solid hold for the hand in contact with the rollator 50 to assure the stability and safety of the user. The rollator should not roll away under the hand in contact therewith. If the wheel braking assemblies 300 are not configured in the braking configuration, this might result in instability and potential falls. Furthermore, when walking, the user should have both hands with firm hold to the rollator 50.

FIG. 12 is showing the reaction of the wheel braking system 650 of the rollator 50 having the wheel braking assembly controller 600 of the first embodiment (FIGS. 9A to 9D) to the above-described conditions. To activate the rollator motion, i.e. configure the wheel braking assemblies 300 into the displacement configuration, each one of the handgrips 204 should be tilted from their upper (raised) configuration 900 (braking configuration) to their horizontal (lowered) configuration 902 (handgrip walking/displacement configuration). When doing so the handgrip control cables 209 transfer the movement to the corresponding attachment points 602, 603 of the balancer plate 623. When only one handgrip 204 is configured into the horizontal (lowered) configuration 902 (in embodiment shown in FIG. 12, the left handgrip 204), then the movement is transferred only to the left attachment point 603 of the balancer plate 623 via the left handgrip control cable 209 which is pulled upwardly along arrow 903. If the right handgrip 204 is not configured into the horizontal (lowered) configuration 902, then the balancer plate 623 pivots about its central pivot 601, resulting in no pulling action on the brake control cables 309. Therefore, the wheel braking assemblies 300 remain in the braking configuration and the rollator 50 cannot be displaced.

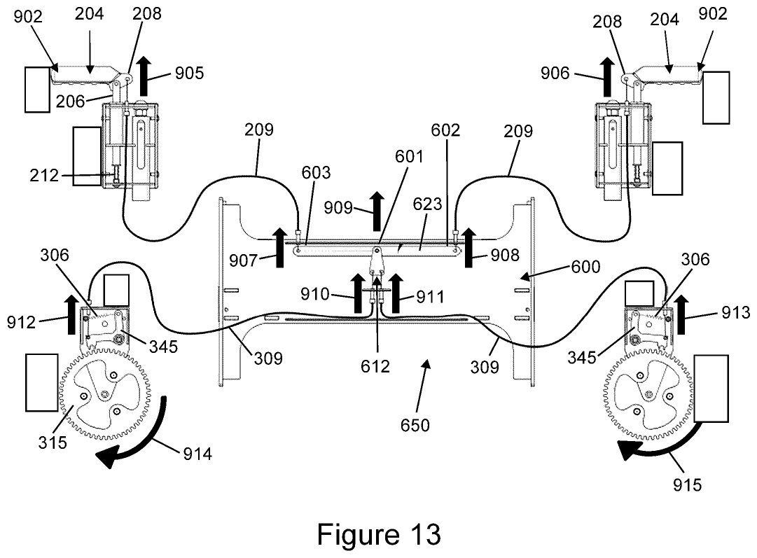

FIG. 13 is showing the reaction of the wheel braking system 650 of the rollator 50 when both handgrips 204 are configured from the raised configuration 900 (handgrip braking configuration) to their horizontal, lowered configuration 902. When these conditions are met and that no excessive pressure is applied on the handgrips 204, then the handgrips 204 are pulling, along arrows 905, 906, the handgrip control cables 209 in a manner such that both ends 602, 603 of the balancer plate 623 are pulled up simultaneously along arrows 907, 908. Then, the balancer plate 623 remains substantially horizontal and pulls the safety condition floating transmission plate 612 upwardly along arrow 909, which in turn pulls up, along arrows 910, 911, the brake control cables 309. Pulling the brake control cables 309 in turn pull up both brake stoppers 345, along arrows 912, 913, which disengage the wheel braking assemblies 300, i.e. the wheel braking assemblies 300 are configured in the displacement configuration. The rear wheels 102 are then free to rotate above their respective rotation axes as shown by arrows 914, 915, either clockwise or counterclockwise. Under these conditions, the rollator 15 is free to be displaced. This situation occurs when both hands are in contact with a respective one of the handgrips 204 and that the rollator user is not unstable.

Linking the handgrip movements with the wheel braking assemblies 300 alone will not prevent the user from falling. Typically, the reflex of a person falling is to transfer its body weight to his hands trying to retake control over his legs failing. As detailed above, the handgrips 204 include a mechanism that detects an abnormal pressure being applied thereon and the wheel braking assembly controller 600 reacts accordingly.

Returning to FIG. 11, on the right side of the flowchart, when the first set of conditions are met in a manner such that the two handgrips 204 are in their horizontal position 902, i.e. the lowered configuration, then the wheel braking system 650 will detect if an abnormal and excessive pressure is applied on the rollator handgrips 204, i.e. the system is configured in the excessive pressure configuration (at least one of the handgrips 204 is configured in the lowered configuration and the pressure applied thereon is equal to or above a handgrip pressure threshold). If excessive/abnormal pressure is detected on one or both handgrips 204, then the wheel braking assembly controller 600 will interpret these conditions as a “type C hazard.” In a “Type C hazard”, the typical behavior of a person falling or losing his balance is detected, i.e. the body weight is transferred onto the user's hands which take hold and compensate the legs weaknesses.

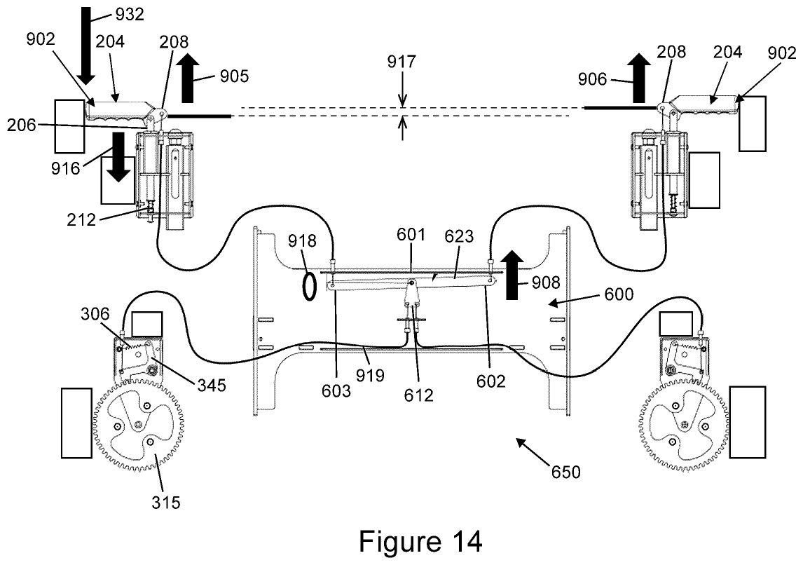

Referring now to FIG. 14, there is the reaction of the wheel braking system 650 of the rollator 50 having the wheel braking assembly controller 600 of the first embodiment (FIGS. 9A to 9D) to the above described conditions, i.e. excessive pressure is applied on at least one of the handgrips 204. In this representation, the two handgrips 204 are shown in their horizontal, lowered configurations 902. When excessive pressure, shown by arrow 932, is applied on the left handgrip 204, i.e. the pressure exceeds the adjustable resistance of the compression spring 212, the rod weight transmitter 206 is translated downwardly along arrow 916 by a given distance 917. The two movements in opposed directions, represented by arrows 905, 916 (i.e. the downward pivoting movement of the handgrip 204 and the translation of the rod weight transmitter 206 in opposed directions), are combined so that the resulting movement is void. Therefore, the left end 603 of the balancer plate 623 remains at its original position 918 while the right end 602 of the balancer plate 623 is being pulled upwardly along arrow 908. Therefore, the balancer plate 623 only pivots about pivot point 601 and no traction is applied on the braking cables 919. Consequently, the wheel braking assemblies 300 remain in the braking configuration.

In addition, the rollator 50 can be used for a person walking in a step by step motion, typically older users or users being re-educated to walk are showing this behavior. In this behavior, the user takes firm hold to the rollator 50 and transfers at least partially his weight to the rollator 50, then drags his feet forward and repeat this sequence. With the mechanism detecting abnormal pressure being applied on the handgrips 204, the rollator 50 will automatically engage the wheel braking assemblies 300 whenever the body weight is transferred to the rollator 50.

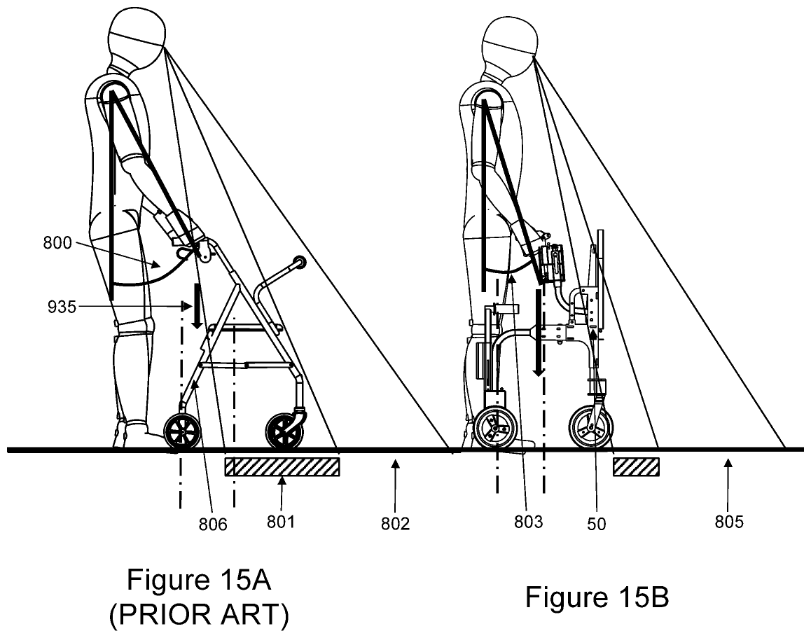

Referring now to FIGS. 15A and 15B, there is shown a comparison of the blind area and the field of view for a rollator user in a walking mode between a typical rollator 806 and the rollator 50 described herein. As it can be seen, a person using a typical rollator 806 is positioned behind a rollator chassis having a wider arm to body angle 800. With the rollator 50, the user is located inside the U-shaped chassis 100 with a smaller arm to body angle 803. The smaller arm to body angle 803 with the rollator 50 should provide a better weight transmission to the rollator chassis 100 together with an improved lateral support, providing a higher stability. In addition, when comparing the field of view 802, 805, the blind area with the rollator 50 should be smaller than the blind area 801 for a typical rollator 806. In addition, the driving position with the rollator 50 should provide a clearer field of view 805 which is important for obstacle identification.

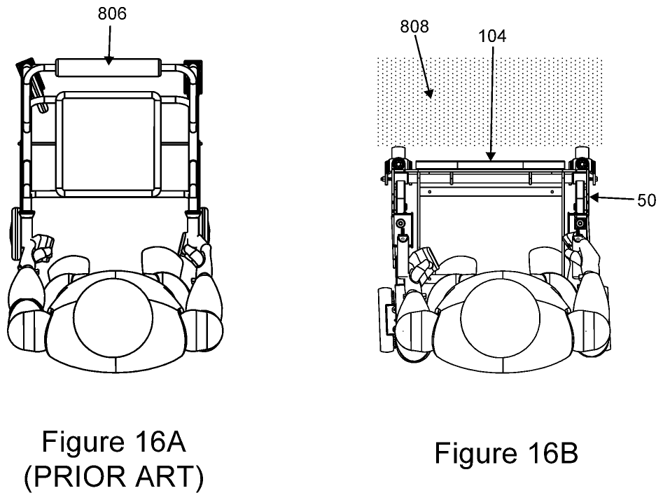

Turning now to FIGS. 16A and 16B, there is shown that the rollator 50 has a smaller footprint 808 than a typical rollator 806. Due to the unobstructed U-shaped chassis 100, the user can enter deeper into the seat/walker receiving area to take hold on the front handrail 104, which may be desirable when a user is driving into crowded restraint environments, such as and without being limitative an elevator. When entering the chassis 100 and grabbing the front handrail 104, the person occupies about half the space than the same person using a typical rollator 806.

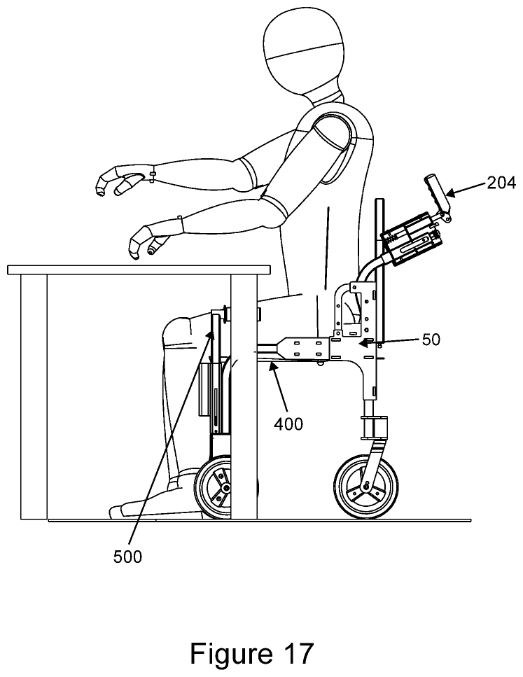

Referring now to FIG. 17, there is shown that the chassis 100 of the rollator 50 can be rolled under a table with a user seated on the seat assembly 400. The primary handgrips 400 can be configured in the frontward configuration. The secondary handgrips 500, located on both sides, can be seized by the user if he attempts to stand up. As detailed above, in the seated configuration with a user seated on the seat assembly 400, the rollator 50 can move freely since the wheel braking assemblies 300 are configured in the displacement configuration. If the user attempts to stand up, then the wheel braking assemblies 300 of the rollator 50 will be automatically configured in the braking configuration and the user will be able to use the secondary handgrips 500 to take hold while standing up.

As shown in FIG. 18, working at a counter surface 809 for meals preparation or other office work related activities is another activity where the rollator chassis 100 is useful. When working at a counter 809 with a typical rollator, the rollator typically is positioned behind the user giving no lateral support and becoming a tripping hazard to the user together with occupying a large space behind him that leads to space congestion. With the rollator 50, the user can position himself close to the counter 809. By default, the rollator 50 is configured in the braking configuration and the user can take hold on the front handrail 104 while working at the counter 809. Being positioned inside of the chassis 100, the user benefits from lateral and frontal support whenever needed.

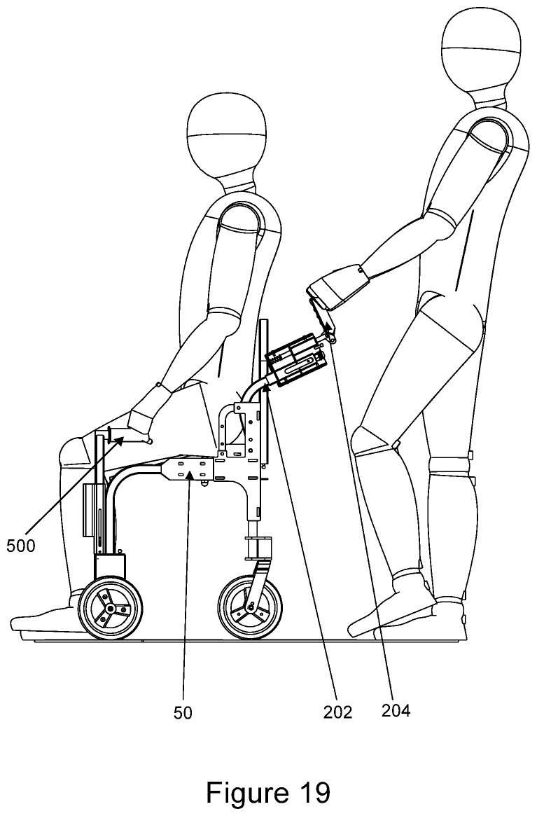

Turning now to FIG. 19, there is shown that the handgrip frames 202 can be pivoted forwardly in the frontward configuration. When the handgrip assemblies 400 are configured in the frontward configuration, the rollator 50 can be used as a transport chair. As described above, when the user is seated on seat 410 of the rollator 50, the wheel braking assemblies 300 of the rollator 50 are configured in the displacement configuration and the rollator 50 is free to be displaced. Therefore, the user can propel himself with his feet. A caregiver can also push the rollator 50 using the handgrips 204. The handgrips 204 are extending forwardly, providing enough frontal space to the caregiver to prevent his knees from interfering with the rollator chassis 100. While being rolled away, the user can take hold on the secondary handgrips 500.

Referring to FIG. 20, when using the rollator 50 in the transport chair mode, optional foldable footrest 700 can be mounted to the accessories receiving tubes 103. In the embodiment shown, the foldable footrest 700 comprises a tubular frame 701 insertable into the accessories receiving tubes 103. A pivot rod 703 is pivotally mounted to a lower end of the tubular frame 701, extends substantially normal to the tubular frame 701, and protrudes therefrom. A footrest plate 702 is mounted on the pivot rod 103 in a manner such that it can be pivoted into a vertical configuration to clear the inside section of the chassis 100. The pivoting movement of the footrest plate 702 on the pivot rod 703 is limited in a manner such that the footrest plate 703 is configured in a substantially horizontal configuration when used, i.e. substantially parallel to the ground. With the footrests 700 in place and the user feet resting thereon, the caregiver can push the rollator 50 with the feet of the user being spaced-apart from the ground, i.e. without dragging thereon.

In FIG. 21, there is shown that the rollator 50 can be used in a conventional and non-adapted toilet stall. The rollator 50 chassis height 811 (FIG. 1) is sufficient for the rollator 50 to be rolled or parked over a toilet 810 with the wheels 102, 128 rolling on each side of the toilet bowl. Once located above the toilet, the user pivots the handgrips 204 forwardly into the frontward configuration. In an embodiment, the handgrips 204 fit on each side of the toilet reservoir 810 and allow the user to get access to the secondary handgrips 500. The secondary handgrips 500 can be pre-adjusted at an optimal height for the user to take hold on them while sitting down on the toilet 810. While sitting down, the rollator 50 is automatically configured in the braking configuration to provide a firm and solid hold to the user.

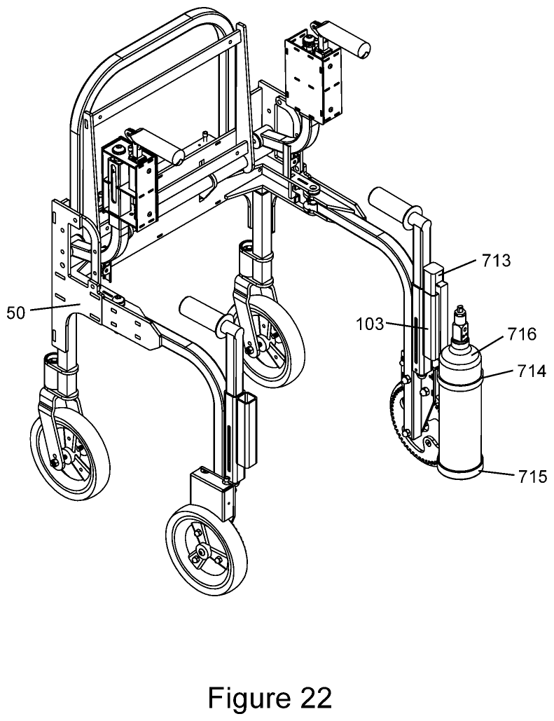

Referring now to FIG. 22, there is shown an embodiment wherein the rollator 50 is equipped with an oxygen tank support. With the accessories receiving tubes 103, the oxygen tank support can be installed and removed quickly and without any or substantial technical assistance. In the embodiment shown, the oxygen tank support comprises an oxygen tank frame 713, engageable in one of the accessories receiving tubes 103, and having a bottom plate 715 and an upper support ring 714. The oxygen tank 716 is slid in the support ring 714 and supported by the bottom plate 715.

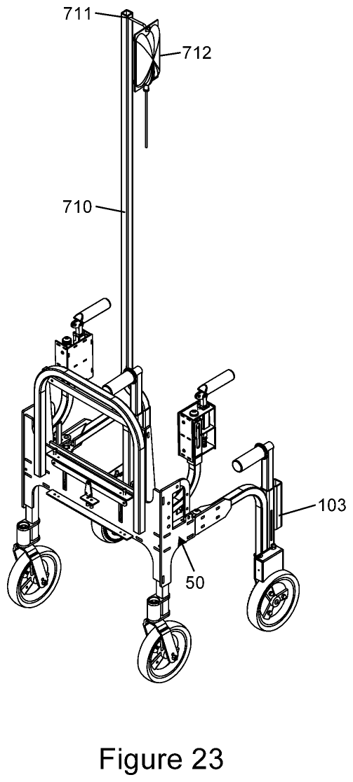

Referring to FIG. 23, there is shown an embodiment wherein the rollator 50 is equipped with a solute poll 710. As for the oxygen tank support, the solute poll 710 can be installed or removed quickly and without any or substantial technical assistance. The solute poll includes a frame 710, insertable in one of the accessories receiving tubes 103, with a perpendicular and horizontal hook 711 to hang the solute pouch 712.

FIG. 24 shows another illustration of a typical accessory that can be fitted onto the rollator 50 in respect with the physical conditions of the user and, more particularly, a walking re-education assembly providing weight support for physiotherapy training.

The walking re-education assembly 760 includes a frame 762 removably engageable with the chassis 100 and a ring 707. The frame 762 has two lateral side portions 764 spaced-apart from one another and extending substantially parallel to a respective one of the lateral side portions 195 of the chassis 100 when engaged therewith. The ring 707 is attached to the two lateral side portions 764 of the frame 762 and extends inbetween. The ring 707 is configured to partially support a weight of the user during a rehabilitation process.

Each one of the two lateral side portions 764 of the frame 762 has a wheel 705 rotatably mounted thereto. In the embodiment shown, the wheels 705 are swiveled wheels so that they do not limit the movement of the rollator 50.

Each one of the lateral side portions 764 has a lower horizontal structural member 718 with a vertical extension insertable in one of the accessories receiving tubes 103, two vertical members: a first one located upfront 717 that extends under the lower horizontal structural member 718 and a second one 704 located rearwardly. The swivel wheel 705 is rotatably mounted to a lower end of the second vertical member 794. A horizontal rail 709 connects the upper ends of the vertical members 704, 717. In the embodiment shown, the vertical members 704 and 717 are bent toward the outside of the rollator chassis 100 so that the rails 709 do not interfere with the user hands, and with the handgrip operation.

A twist belt 706 is connected to the outside ring 707 with two pivots coaxially located on each side of the belt 719, 730. The combination of the twist belt 706 and the pivots allow the transmission of the user weight to the outside ring 707 with the possibility for the twist belt 706 to tilt frontward and rearward in order to follow the hip movement of the user while walking or sitting down.

In an embodiment, the outside ring 707 is made of a concave shaped track with the concavity facing the outside of the ring 707. Two chariots 708, located on each side of the user, are linking the outside ring 707 with the longitudinal supporting rails 709. Each one of the chariots 708 comprises three sections. A first section (on the illustration the left end of it) has two or more wheels including bearings that travel inside the outside ring 707. The wheels are placed on a compatible diameter and specially designed chariot that allows the wheels to travel inside the outside ring 707 with minimal friction or effort from the user. A second section of the chariot (on the illustration the right end) is equipped with two or several wheels that travel linearly inside the lateral rails 709 of the lateral side portions 764 of the frame 762. Both sections are linked together with a link that can extend but can retain the weight of the user. This link could be, for example, a rubber band with sufficient extension in a manner such that the user can sit on the rest seat 410 but cannot touch the ground if he falls. The link could also be made from a device that rolls/unrolls a belt but blocks the rolling movement when a sudden movement is made. For instance, it could be similar to a car seatbelt system. In his example, the use of a seat belt retractable/blocking mechanism would give the greatest freedom of movement to the user. The middle section of the chariot 708 could also use a retractable/blocking mechanism that could integrate an adjustable feature that would allow the mechanism to support between 20 to 100% of the user bodyweight.

Both the outside ring track and the first section of the chariot 708, which are interacting together, should be made in a manner such that the chariot cannot roll out of the ring 707 under any circumstances to assure safety of the user. The lateral rails 709 should have a locking system that does not permit the second section of the chariot 708 to go out of the lateral rails 709. In addition, this locking system should allow the removal of the twist belt from the lateral rails 709 to ease the installation of the twist belt 706 on the user.

Turning now to FIGS. 26A to 26C and 27A and 27B, there is shown that the rollator 50 can be provided with two sets of rear wheels. A first set of rear wheels 102 is characterized by a relatively small diameter as shown for instance in FIGS. 1A to 1C and a second set of rear wheels 001 is characterized by a wider diameter as shown in FIGS. 26A to 26C. With the second sets of rear wheels 001, the rollator 50 can be used as a wheelchair and can be propelled manually through push rims 042. All the features described above still apply to the configuration of the rollator with the second sets of rear wheels 001 and will not be repeated.

It is however appreciated that the configuration of the wheel braking assembly can vary, as shown in FIGS. 27A and 27B. Furthermore, in an embodiment, the shape and configuration of the lateral side portions of the chassis should also be modified to receive the wider diameter wheels 001.

In the embodiment shown, each one of the lateral side portions 043 of the chassis is provided with a L-shaped square tube 002 having hinge plates 003, 004, 005 mounted at a front end thereof. The lateral side portions 043 of the chassis are pivotally mounted to the front portion of the chassis through the hinge plates 003, 004, 005 in a manner such that the chassis is configurable in the folded configuration and the operative configuration described above. More particularly, the hinge plates 003, 004, 005 are pivotally mounted to lateral plates 129 at pivoting axis 006.

On its lower end, the L-shaped square tube 002 has a triangular shaped plate 009 mounted (welded or assembled) thereto. The triangular shaped plate 009 has a round or oblong opening defining therein at a relatively centered position that permits the installation of an axle tube 010. This axle tube 010 can be welded in place at a fixed position or can be made of an outside threaded tube with two opposite flat faces that are compatible with an oblong opening cut at the triangular axle attachment plate 009. Using a threaded axle tube 010 would allow to adjust the width of the wheels to fit the user's needs. If a threaded axle tube 010 is used, this tube would be secured in place using an appropriate mechanical fastener(s), such as nut(s) 011. The wheelchair wheels 001 can then be rotatably mounted to the lateral side portion 043 of the chassis with the wheel axle 012 being a fixed wheel or a quick release type of wheel.

Hereinbelow, since both lateral side portions 043 are similar, only one will be described. However, it is appreciated that the description applies to both lateral side portions.

The lateral side portion 043 of the chassis is provided with a triangular plate 007 located above the shaped square tube 002 for the attachment of the height adjustable handgrip receiving tube 505 of the secondary handgrips 500 on which is attached or welded the accessories receiving tubes 103 in which a plurality of accessories can be selectively received.

Still referring to FIGS. 27A and 27B, there is shown that the L-shaped square tube 002 has attachment points 008 located on the side thereof, close to a center and at mid-height. The location of the attachment points 008 can differ depending of the size (diameter) of the wheels to be mounted to the rollator and with the configuration of a wheel braking assembly 015. In the embodiment shown, two attachment points 008 are shown but it is appreciated that the number and the configuration of the attachment points can vary.

The wheel braking assembly 015 will be described in reference to FIG. 27B. In the embodiment shown, the wheel braking assembly 015 includes a gear braking disk 013 attached to the wheelchair wheel 001. The gear braking disk 013 is attached using pre-cut openings 021 defined on the outside diameter of the gear braking disk 013 to the existing push rim attachment points 021 of the wheelchair wheel 001 using mechanical fasteners, such as screws. The gear braking disk 013 includes a plurality of teeth 014 located on an inside diameter thereof and extending toward the center of the wheels 001. The teeth 015 can have any suitable geometry, such as and without being limitative the one described above in reference to FIG. 25.

The wheel braking assembly 015 includes a plastic cast body 051 in which a wheel stopper 016 with corresponding rounded teeth travels from a braking configuration to a displacement configuration. In the embodiment shown, the wheel stopper 016 travels linearly between the two configurations. A brake control cable 019 operatively connected to the wheel braking assembly controller 600 travels into a sheath 020, such as a Teflon® line sheath, and is secured in place at both ends. The brake control cable 019 is attached to a movement transmission pulley 017 of the wheel braking assembly 015 at pulley attachment point 018.

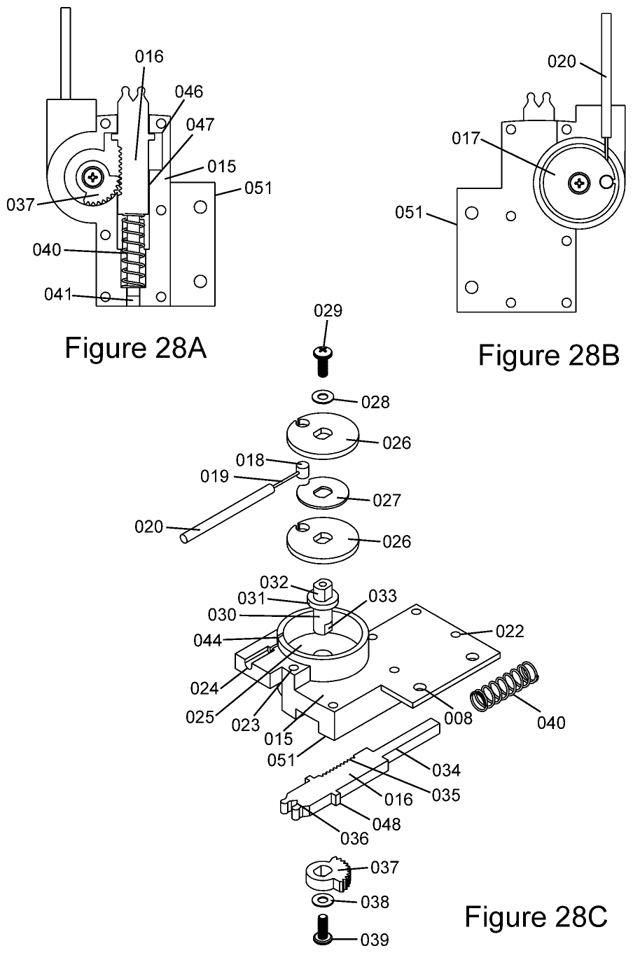

Referring to FIGS. 28A to 28C, detailed views of the wheel braking assembly 015 are shown. In an embodiment, the wheel braking assembly 015 is made of the plastic casting body 051 having two attachment points 008 that allow the wheel braking assembly 015 to be attached to the lateral side portion 043 of the chassis.

In the embodiment, the body 051 is provided with a plurality of attachment points 022 for securing a main cover (not shown) to cover components of the wheel braking assembly 015. The components of the wheel braking assembly 015 include, amongst others, a transmission gear 037, a linear braking lever 016, and a compression spring 040. The body 051 also includes a sheath cavity 024 where the sheath 020 is inserted and compressed into place using a compression plate (not shown) in order to prevent the sheath 020 from moving. The compression plate (not shown) is secured in place using one or many attachment point(s) 023.

The back side of the body 051 is provided with a cylindrical cavity 025 where a cable attachment pulley assembly 017 is inserted. The cylindrical cavity 025 has a relatively tangential and linear opening 044 in which the brake control cable 019 is inserted and travels.

The front side of the body 051 is provided with a rectangular cavity divided into several sections. The braking stopper 016 is provided with side stoppers that are contained in a first top part 046 of the cavity. The side stoppers travel inside this first portion 046 of the cavity and limit the motion of the braking lever 016 between a maximum extension and a minimum extension. A middle part 047 of the cavity has a width slightly larger than a width of a main body of the braking stopper 016. The middle part 047 of the cavity is designed to guide the braking stopper 016 into a linear displacement, i.e. a translation inside the cavity. A third part of the cavity is designed to receive the compression spring 040 therein. Finally, a channel 041 having a narrow section is designed to receive an end of the brake stopper 016 to ensure its linear displacement.

The cable attachment pulley assembly 017 includes two round side plates 026 having at their center an oblong opening that allows the pulley assembly 017 to transmit the linear motion of the brake control cable 019 into a linear motion but oriented in an opposite direction to the motion transmission gear 037 and to the braking stopper 016 located on the opposite side of the body 051. The two side plates 026 also have a cylindrical opening close to their perimeter where the end of the brake control cable 018 can be inserted. The pulley assembly 017 also includes a round middle plate 027 that maintains the two side plates 026 spaced-apart from each other. The thickness of the middle plate 027 is slightly thicker than a diameter of the brake control cable 019 in a manner such that the cable 019 can travel freely between the two side plates 026. The cable attachment pulley 017 is secured to the transmission shaft 030 with the use of a mechanical fastener, such as a screw 029 tighten over a flat washer 028.

A top portion 032 of the transmission shaft 030 has a geometry compatible with the center opening of the pulley assembly 017, which are in the embodiment shown oblong in shape. The transmission shaft 030 also has a cylindrical middle portion 031 showing an enlarged diameter that allows the shaft 030 to be positioned at the proper and exact height. The transmission shaft 030 is inserted into a hole defined in the center of the cylindrical opening 025 of the body 051. At the lower portion of the transmission shaft 030, a rounded section with two flat surfaces 033 are machined and correspond to the center opening of the gear motion transmitter 037. The gear motion transmitter 037 includes a partial gear section with a centered opening corresponding in shape to the transmission shaft 030. The gear motion transmitter 037 is secured to the transmission shaft 030 using a flat washer 038 and a mechanical fastener, such as a screw 039.

The brake stopper 016 has at one end one, two or a plurality of rounded teeth 036 having a shape designed to be complementary in shape with the teeth of the gear disk 013 to be engageable and disengageable therewith with low interference. The braking stopper 016 has two stoppers 048, on each side of the main body, that interfere with the top section cavity 047 of the body 015. It contains the displacement of the stopper 016 between the maximum position and the minimum position. The braking stopper has on one side a cogwheel or gear shape 035 corresponding in geometry with the motion transmission gear 037 transforming the rotational motion of the gear 037 into a linear motion for the brake stopper 016. Finally, the brake stopper 016 has a smaller section 034 at its other end that allows for the insertion of the compression spring 040. The compressions spring 040 biases the brake stopper 016 into to the brake configuration whenever the safety conditions are not met.

In the above paragraphs, there was the description of a wheel braking assembly for the wheelchair configuration of the rollator 50. It is obvious that the same linear movement of the brake stopper can be obtained with the use of a parallel lever. It is also understood that the teeth of the brake gear can be oriented outwardly, i.e. towards the outside of the wheel.

It is also appreciated that the configuration of the wheel braking assembly and the frame can vary from the embodiments described above.

The wheel braking assembly controller 600 acts as a fall detection system that detects the behavior of the user when he/she is about to fall and to apply the wheel breaking assemblies 300 automatically in a way to give a solid hold to the user. Additionally, in an embodiment, the rollator 50 is designed in a manner such that the wheel braking system 650 is automatically configured in the braking configuration whenever the user does not have his two hands in contact with the two handgrips 204. This additional function is useful for example when the user takes hold on the rollator 50 while bending over to pick up an item in the refrigerator or an object on the ground. This function also helps teaching the user to keep both hand on the handgrips 204 while walking.

As detailed above, the wheel braking system 650 is operatively connected to the seat 410 (through the wheel braking assembly controller 600) and configured in a manner such that the wheel braking system 650 is configured in the displacement configuration when the rollator user is seated on the seat 410, i.e. a pressure equal to or greater than a seat pressure threshold is applied on the seat 410. When the rollator user stands up, i.e. the pressure applied on the seat 410 is below the seat pressure threshold, the wheel braking system 650 is automatically configured in the braking configuration. The rollator user can therefore propel himself with his feet while being seated. It also provides an anti-roll back system, i.e. the wheel braking system 650 is automatically configured in the braking configuration when the rollator user attempts to stand up. This function may be particularly helpful for user suffering from memory impairments such as Alzheimer or mental dysfunctions or simply distraction.

The wheel braking system 650 in combination with the wheel braking assembly controller 600 is configured to determine/detect whenever the rollator user is at risk of a fall and automatically configures the wheel braking system 650 in the braking configuration.

The rollator chassis 100 is designed to be a relatively lightweight chassis and, therefore, be adapted to be used in everyday life. As detailed above, by being relatively compact and configurable in a further compacted configuration, the rollator 50 is adapted to a non-adapted rollator environment in order to permit full accessibility to existing public environment. For example, the rollator 50 can be rolled over a public toilet. The secondary handgrips allow the rollator user to use public, non-adapted toilets with ease and comfort.

The rollator 50 can be adaptable to a physical or medical condition of the user by including one or more accessory receiving tube 103 to receive and support optional accessories such as and without being limitative: foot rest supports 702, cup holders, solute supports, oxygen tank supports, training tools such as walking re-education assembly 760, cup holders, telephone holders, electronic tablet holders or any commodity accessories the user may need. In the embodiments shown the accessory receiving tubes 103 are mounted to the rear ends of the chassis 100. However, it is appreciated that they can be mounted to any other suitable location on the chassis 100.

It is appreciated that, in an alternative embodiment (not shown) slowing down brakes can be mounted to the handgrips 204 and can be used by the caregiver while pushing the rollator 50 in its transport chair configuration or by the user itself when using in the rollator 50 in the walking mode to slow down its displacement.

The chassis construction allows the rollator to be used in a non-adapted environment such as public toilets, elevators, kitchen counter, office desks, etc. The chassis construction allows the user to equip the rollator 50 with many useful features such as oxygen tank support, solute poll, loading basket and body weight support device. The main philosophy behind the rollator 50 being that the user carries a safe and adapted environment with him and that the health professional can equipped the rollator 50 to use it as a re-education tool.

The rollator 50 can be advantageously inserted in the rollator, walker, transport chair and adapted training tool markets.

Several alternative embodiments and examples have been described and illustrated herein. The embodiments of the invention described above are intended to be exemplary only. A person of ordinary skill in the art would appreciate the features of the individual embodiments, and the possible combinations and variations of the components. A person of ordinary skill in the art would further appreciate that any of the embodiments could be provided in any combination with the other embodiments disclosed herein. It is understood that the invention may be embodied in other specific forms without departing from the central characteristics thereof. The present examples and embodiments, therefore, are to be considered in all respects as illustrative and not restrictive, and the invention is not to be limited to the details given herein. Accordingly, while the specific embodiments have been illustrated and described, numerous modifications come to mind. The scope of the invention is therefore intended to be limited solely by the scope of the appended claims.

1.-136. (canceled)

137. A rollator for mobility assistance to a user, the rollator comprising:
a chassis including:
a front portion and
two lateral side portions,
each one of the two lateral side portions extending rearwardly from a respective end of the front portion;
at least one right-side wheel and at least one left-side wheel rotatably mounted to at least one of the front portion of the chassis and rear ends of the lateral side portions of the chassis;
at least a right-side and a left-side wheel braking assemblies, each one of the right-side and left-side wheel braking assemblies having a wheel stopper engageable with a respective one of the right-side and left-side wheels and configurable in a displacement configuration allowing rotation of the respective one of the right-side and left-side wheels and a braking configuration wherein the wheel stopper is engaged with the respective one of the right-side and left-side wheels and prevent rotation thereof; and
a pair of pivotable handgrips operatively connected to the wheel braking assemblies and being selectively configurable in a handgrip displacement configuration and in a handgrip braking configuration, wherein configuration of one of the handgrips in the handgrip braking configuration configures the wheel stoppers of the right-side and left-side wheel braking assemblies in the braking configuration wherein they are engaged with their respective one of the right-side and left-side wheels.

138. The rollator according to claim 137, wherein the at least one right-side wheel and at least one left-side wheel comprises a right-side fixed wheel and a left-side fixed wheel and the wheel stopper of the at least right-side wheel braking assembly is engageable with the right-side fixed wheel and the wheel stopper of the at least left-side wheel braking assembly is engageable with the left-side fixed wheel, wherein the right-side fixed wheel and the left-side fixed wheel are rotatably mounted to a respective one of the rear ends of the lateral side portions of the chassis.

139. The rollator according to claim 137, wherein the right-side and the left-side wheel braking assemblies are configured in the displacement configuration if both handgrips are configured in the handgrip displacement configuration.

140. The rollator according to claim 137, wherein
the chassis defined by the front portion and the two lateral side portions has a substantially U-shape profile with the front and lateral side portions defining a seat/walker receiving area inbetween; and
the rollator further comprises a seat assembly including a seat pivotally mounted to the chassis and selectively configurable in a raised configuration and a seated configuration wherein the seat extends substantially horizontally in the seat/walker receiving area, the seat/walker receiving area being unobstructed when the seat is configured in the raised configuration.

141. The rollator according to claim 140, wherein the lateral side portions are pivotally mounted to the respective end of the front portion and selectively configurable in an operative configuration wherein they are spaced-apart from one another to define the seat/walker receiving area inbetween and a compacted configuration wherein they are superposed to each other and extend substantially parallel to the front portion.

142. The rollator according to claim 141, wherein the seat is configured in the raised configuration in the compacted configuration of the lateral side portions and is located between the front portion and the lateral side portions of the chassis.

143. The rollator according to claim 137, wherein the handgrips are pivotable and:
the handgrip displacement configuration comprises a lowered configuration in which the handgrips extend substantially horizontally and wherein a pressure below a handgrip pressure threshold is applied thereon, and wherein the wheel braking assemblies are disengaged from their respective wheels if both handgrips are simultaneously configured in the handgrip displacement configuration; and
the handgrip braking configuration comprises:
a raised configuration of the handgrips wherein the handgrips extend above the handgrip displacement configuration and a lowerable into the handgrip displacement configuration; and
a excessive pressure configuration wherein the handgrips are configured in the lowered configuration and a pressure equal to or greater than the handgrip pressure threshold is applied thereon.

144. The rollator according to claim 143, wherein the handgrips are configured in the raised configuration when no pressure is applied thereon.

145. The rollator according to claim 140, further comprising a wheel braking system comprising the wheel braking assemblies and being operatively connected to the seat of the seat assembly, wherein when the seat is configured in the seated configuration with a pressure greater than or equal to a seat pressure threshold applied thereon, the wheel braking assemblies are configured in the displacement configuration and when the seat is configured in the seated configuration with a pressure lower than the seat pressure threshold applied thereon, the wheel braking assemblies are configured in the braking configuration.

146. The rollator according to claim 145, wherein the wheel braking system further comprises a wheel braking assembly controller mounted to the chassis and operatively connecting the handgrips to the wheel braking assemblies.

147. The rollator according to claim 146, wherein the wheel braking assembly controller comprises:
a pair of outer tubes, each one of the outer tubes being operatively connected to a respective one of the handgrips and being rotatable along a rotation axis thereof upon actuation of the respective one of the handgrips;
a shaft operatively connected to the wheel braking assemblies, the shaft having a substantially cylindrical portion at least partially enclosed into the outer tubes and a joint protruding from the cylindrical portion, the shaft being rotatable along a rotation axis thereof to configure the wheel braking assemblies simultaneously in one of the braking configuration and the displacement configuration, wherein the outer tubes abut against the joint when rotating simultaneously to engage the shaft in rotation and wherein the shaft is not engaged in rotation when only one of the outer tubes is engaged in rotation.

148. The rollator according to claim 146, wherein the wheel braking assembly controller comprises: a power supply, handgrip sensors operatively connected to the handgrips, a logic controller, electric connectors connecting the handgrip sensors to the wheel braking systems through the logic controller, and a power supply operatively connected to at least one of the handgrip sensors, the logic controller and the wheel braking assemblies to supply electric power thereto.

149. The rollator according to claim 137, further comprising a handgrip assembly comprising at least one handgrip frame pivotally mounted to the chassis and including lateral side portions extending substantially in a same plane than a respective one of the lateral side portions of the chassis, each one of the lateral side portions of the at least one handgrip frame having one of the pair of handgrips pivotally mounted thereto, the at least one handgrip frame being selectively configurable in an operative configuration wherein at least one of the handgrips is located rearwardly of the front portion of the chassis and a frontward configuration wherein at least one of the handgrips is located forwardly of the front portion of the chassis.

150. The rollator according to claim 137, wherein each one of the wheel braking assemblies comprises a gear disk secured to the respective one of the wheels, the gear disk having a plurality of triangular-shaped teeth extending peripherally and the respective one of the wheel stoppers is engageable with the respective gear disk in the braking configuration to prevent rotation of the respective one of the wheels.

151. The rollator according to claim 150, wherein each one of the gear disks includes a plurality of rounded recesses, adjacent ones of the triangular-shaped teeth being separated by one of the rounded recesses defined inbetween.

152. The rollator according to claim 150, wherein each one of the wheel stoppers comprises a tooth engaging brake lever pivotally mounted to the chassis and having at least one round-ended tooth.

153. The rollator according to claim 152, wherein the at least one round-ended tooth comprises at least a pair of round-ended teeth, each one of round-ended teeth being spaced-apart by a pointed tooth receiving recess.

154. A rollator for mobility assistance to a user, the rollator comprising:
a chassis including:
a front portion and
two lateral side portions,
each one of the two lateral side portions extending rearwardly from a respective end of the front portion;
at least one right-side wheel and at least one left-side wheel rotatably mounted to at least one of the front portion of the chassis and rear ends of the lateral side portions of the chassis;
at least a right-side and a left-side wheel braking assemblies, each one of the right-side and left-side wheel braking assemblies having a wheel stopper engageable with a respective one of the right-side and left-side wheels and configurable in a displacement configuration allowing rotation of the respective one of the right-side and left-side wheels and a braking configuration wherein the wheel stopper is engaged with the respective one of the right-side and left-side wheels and prevent rotation thereof; and
a pair of pivotable handgrips operatively connected to the wheel braking assemblies and being selectively configurable in a handgrip displacement configuration and in a handgrip braking configuration, wherein the wheel stoppers of the right-side and left-side wheel braking assemblies are configured in the displacement configuration allowing rotation of the respective one of the right-side and left-side wheels unless the two handgrips are configured in the handgrip displacement configuration.

155. A rollator for mobility assistance to a user, the rollator comprising:
a chassis including:
a front portion and two lateral side portions, each one of the lateral side portions extending rearwardly from a respective end of the front portion to define a seat/walker receiving area;
at least one pair of wheels rotatably mounted to the chassis; and
a handgrip assembly including a handgrip frame pivotally mounted to the chassis and including a lateral side portion extending substantially in a same plane than a respective one of the lateral side portions of the chassis, the lateral side portion of the handgrip frame including a handgrip mounted thereto, the handgrip frame being configurable in an operative configuration wherein the handgrip is located rearwardly of the front portion of the chassis and a frontward configuration wherein the handgrip is located forwardly of the front portion of the chassis.

156. The rollator according to claim 155, further comprising a seat assembly and a wheel braking system; the seat assembly including a seat pivotally mounted to the chassis and selectively configurable in a raised configuration and a seated configuration wherein the seat extends substantially horizontally in the seat/walker receiving area, the seat/walker receiving area being unobstructed when the seat is configured in the raised configuration; and the wheel braking system being engageable with the at least one pair of wheels and configurable in a displacement configuration allowing rotation of the wheels and a braking configuration wherein the wheel braking system is engaged with at least one of the at least one pair of wheels to prevent rotation thereof and wherein the seat assembly is operatively connected to the wheel braking system, wherein when the seat is configured in the seated configuration, the wheel braking system is configured in the braking configuration if a pressure applied on the seat is lower than a seat pressure threshold.

157. The rollator according to claim 156, wherein, when the seat is configured in the seated configuration, the wheel braking system is configured in the displacement configuration if a pressure equal to or greater than the seat pressure threshold is applied thereon and the seat is further pivoted downwardly towards a horizontally-extending configuration when a pressure equal to or greater than the seat pressure threshold is applied to the seat; and
the handgrip is pivotally mounted to the handgrip frame and is operatively connected to the wheel braking system, the handgrip being selectively configurable in:
a raised configuration and a lowered configuration, wherein, if the seat is configured in the raised configuration, configuration of the handgrip in the raised configuration configures the wheel braking system in the braking configuration and configuration of the handgrip in the lowered configuration with a pressure applied thereon equal to or greater than a handgrip pressure threshold configures the wheel braking system in the braking configuration.

158. The rollator according to claim 155, wherein the lateral side portions are pivotally mounted to the respective end of the front portion and selectively configurable in an operative configuration wherein they are spaced-apart from one another to define the seat/walker receiving area inbetween and a compacted configuration wherein they are superposed to each other and extend substantially parallel to the front portion.
